(19) United States
(12) Patent Application Publication
(10) Pub. No.: US 2018/0045730 A1
(43) Pub. Date: Feb. 15, 2018

(54) METHODS FOR PREDICTING TUMOR RESPONSE TO TARGETED THERAPIES

(71) Applicant: 20/20 GeneSystems, Inc., Rockville, MD (US)

(72) Inventors: Jonathan Cohen, Potomac, MD (US); Alexandrine Josephe Derrien-Colemyn, Bethesda, MD (US); John Gillespie, Clarksville, MD (US); Soon Sik Park, Clarksville, MD (US)

(73) Assignee: 20/20 GeneSystems, Inc., Rockville, MD (US)

(21) Appl. No.: 15/657,098

(22) Filed: Jul. 21, 2017

Related U.S. Application Data
(63) Continuation of application No. 14/375,827, filed on Jul. 31, 2014, now abandoned.
(60) Provisional application No. 61/593,688, filed on Feb. 1, 2012.
(60) Provisional application No. 61/654,400, filed on Jun. 1, 2012.
(60) Provisional application No. 61/660,305, filed on Jun. 15, 2012.
(60) Provisional application No. 61/660,692, filed on Jun. 16, 2012.
(60) Provisional application No. 61/665,943, filed on Jun. 29, 2012.
(60) Provisional application No. 61/699,760, filed on Sep. 11, 2012.
(60) Provisional application No. 61/699,874, filed on Sep. 12, 2012.

(51) Int. Cl. G01N 33/574; A61K 45/06; A61K 31/436; A61K 38/00

(52) U.S. Cl. CPC G01N 33/57492; G01N 33/574; A61K 31/436; A61K 45/06; A61K 38/00

(57) ABSTRACT

A method for identifying cancer patients that are likely to be responders or non-responders to a signal transduction pathway inhibitor is described.

### BACKGROUND

Traditional approaches to chemotherapy for cancer patients beginning in the 1940s involved administration of various cytotoxic drugs such as alkylating agents, platinating agents, antimetabolites, topoisomerase inhibitors, and other agents designed to kill all rapidly dividing cells in the body. These drugs were highly toxic in nature and, because they are non-targeted, cause side effects such as nausea, hair loss, etc. Beginning in the late 1990s, new types of targeted anticancer agents have been introduced. These include monoclonal antibodies (such as HERCEPTIN®) which target a range of cell surface receptors, and small molecules that interact with various cell signalling pathways (e.g. TORISEL® that targets the mTOR pathway). While these newer targeted therapies avoid some of the toxicity and side effects of older cytotoxic agents, they have tended to be effective in only small subsets of patients to whom the drugs are administered. For example, only about 40% of breast cancer patients derive benefit from HERCEPTIN® while for kidney cancer patients fewer than 30% respond to drugs that target the VEGF pathway (e.g. SUTENT®) while less than 10% benefit materially from mTOR inhibitors such as TORISEL® (temsirolimus). Despite the limited effectiveness for the majority of patients, many of these targeted therapies cost $5000 or more per month over several months.

For the foregoing reasons there is a compelling unmet need for tests to predict whether particular targeted therapies will likely be effective in a particular patient. To meet that need, several attempts have been made to identify and to validate biomarkers that predict sensitivity of a tumor to targeted therapies such as VEGF and mTOR inhibitors. Regarding mTOR inhibitors, in particular, many of those studies met with little success. For example, the study subgroup analyses from the phase 3 global advanced renal cell carcinoma (ARCC) trial (Figlin et al., Cancer (2009) 15:3651-3660) using immunohistochemistry (IHC) to monitor expression of HIF1α and PTEN in kidney cancer tissue from 112 patients treated with temsirolimus revealed that those two markers did not predict response of renal cell carcinoma to temsirolimus therapy.

A factor hampering the identification and validation of predictive biomarkers has been a paucity of technologies for analyzing tumor samples that, for example, preserve tissue morphology so that it can be confirmed that the biomarkers are expressed in the tumor rather than in adjacent non-cancerous tissue (e.g. stroma). Cancer tissue typically is comprised of a variety of different non-cancerous cell types including blood vessels, inflammatory cells, nerve, fibroblasts and so on. To avoid confounding the data with contamination with other non-cancerous cells, it is vital that the biomarker expression be localized specifically to the cancer regions of interest on the tissue. Also, biomarkers may not be detectable or present in more readily accessible tissues, such as, blood.

Another limitation of existing “companion diagnostics” (i.e. diagnostic tests developed to predict response of a particular drug) on the market is that they generally measure only a single analyte or biomarker. For example, as of the end of 2012 the U.S. Food & Drug Administration had approved only 15 companion diagnostics, each of which measures only one of the following genes or biomarkers: HER2, EGFR, KRAS, C-KIT, or ALK. This small number of approved tests, all associated with targeted cancer drugs, comes after more than a decade of intense research effort by academia and industry in the area of “personalized medicine” for oncology.

Cancer growth and spread is dependent on several factors, including activation of signalling pathways that relate to the increased metabolic activity of the growing cells. Many pathways involve phosphorylation or dephosphorylation of components to the pathway to transmit signal. Certain assay, such as, DNA and RNA assays, such as PCR, DNA microarrays and the like generally are incapable of measuring phosphoproteins and/or phosphorylation.

A multiplex biomarker identification technology can be particularly useful for identifying and testing predictive tumor biomarkers since it permits the use of an internal control or combinations thereof and because signalling pathway can comprise more than 10, more than 20, more than 30, or more component molecules, each of which can be diagnostic for a particular cancer or response thereof to a particular treatment. Unfortunately, with scarce or small tumors (e.g. needle biopsies), it is often difficult or impossible to test all candidate biomarkers with conventional techniques, such as, standard IHC.

Finally, a robust assay is one which is operable with tissue that has been routinely fixed in 10% neutral buffered formalin and embedded in paraffin (FFPE), which has been for many years and remains the standard means for preserving tissue in hospital pathology departments throughout most of the world.

Thus, it would be desirable to have a test that would be amenable for use on standard pathology specimens to help predict the responsiveness of patient with a solid tumor to one or more agents designed to target one or more components of the mTOR, VEGF, or other pathways associated with tumor growth or metastasis so as to improve patient outcome and to reduce costs to the healthcare system.

It would also be desirable if such a test could generally preserve the morphology of the tissue to so that the localization of the expressed biomarkers can be established in situ.

It would further be desirable if such a test were run using a multiplex platform so that multiple biomarkers can be assessed even with small tissue samples, such as, core needle biopsies.

It would further be desirable if such a test could be used with tissue that is preserved by FFPE but also by other methods, such as, frozen sections and alcohol-fixed sections.

For the foregoing reasons, there is a need for a test that can help identify tumor responsiveness to agents designed to target pathways associated with tumor growth and metastasis including, without limitation the mTOR and VEGF pathways which are among the most common targets of novel cancer therapies.

### SUMMARY

The present invention is directed to methods and tests that help predict the likelihood that a tumor will respond to a targeted therapy so that those cancer patients most likely to benefit can receive that drug in a timely manner and those patients unlikely to benefit from a particular drug can instead be prescribed alternative therapies. The test comprises a panel of two or more biomarkers that are part of a signal transduction pathway. The biomarkers included in the panel, in combination, express differently in tumors that respond the drug from those that do not respond. The drugs to which these tests predict response may be designed or determined to target the signal transduction pathway of which the biomarker panel is associated. Examples of such drugs are those that target angiogenesis pathways like VEGF or cellular growth pathways like mTOR. Alternatively, the drugs might target something other than the pathway with which the biomarkers are associated but activation of that pathway might otherwise limit or defeat the effectiveness of that drug. Examples of such drugs are those that block cellular receptors but for which activation of downstream signaling pathways nevertheless maintains cellular growth.

The biomarker tests disclosed herein are developed by obtaining a representative number of annotated tumor samples from both responders and non-responders of the drug of interest. In certain embodiments these sets of samples are derived from several different treatment centers to avoid sources of bias, etc. A technology for suitably measuring multiple signaling biomarkers in tissue is, in certain embodiments, employed and the measurements of a large pool of candidate biomarkers from tumors of those who responded to the drug are compared to measurements from non-responding tumors. Those biomarkers which in combination yield the best differentiation are selected to be part of a panel. A score is developed to classify tumors as likely responders or likely non-responders. Other categories such as “indeterminate” can also be created.

In clinical practice, treating physicians test biopsied or resected tumor samples with the subject biomarker panel and create an individual patient score, also referred to herein as the aggregate or predictive score. The patient score is compared to a data set comprising aggregate scores from retrospective samples with a threshold value so that the tumor is classified based on its likelihood of responding to particular targeted therapies. This classification helps the physicians select the targeted therapies most likely to benefit the individual patient.

These and other features, aspects, and advantages of the present invention will become better understood with reference to the following description and appended claims.

### BRIEF DESCRIPTION OF THE DRAWINGS

The numerous advantages of the present invention can be better understood by those skilled in the art by reference to the accompanying figures in which:

FIG. 1A illustrates a scoring method for obtaining an assigned score for a measured biomarker used with layered immunohistochemistry (L-IHC) methods for labeling biomarkers in a sample. In this method the assigned score is obtained by providing the labeled biomarker with an intensity designator (e.g. 1) and then multiplying one (1) by the percentage of the region of interest (ROI, area or areas containing cancer cells) labeled with biomarker having an intensity of three (3) (e.g. 3×0.05 (for 5%)=0.05) and combining that with the product of the intensity (2) and percentage ROI stained (30%) in the other ROI. This is repeated for each different labeled biomarker intensity present on the same membrane and then those numbers (e.g. 0.75) are summed and rounded to the closest integer to obtain the assigned score for each biomarker. The assigned score calculated by this method in this Figure is 1 or zero (0) depending on the membrane. See, Example 1A

FIGS. 1B and 1C illustrate another scoring method for obtaining an assigned score for a measured biomarker used with L-IHC methods for labeling biomarkers in a sample. In this method, the labeled biomarker is provided with an intensity designator (e.g. 0-3) that is multiplied by a graded scale for the percentage of the ROI with labeled biomarker. For example, the ROI with less than 10% of the area with labeled biomarker is designated as one (1); 10% to 50% is designated as two (2); 50% to 80% is designated as three (3) and greater than 80% is designated as four (4). See, Example 1B

FIG. 2 provides a series of images of consecutive membranes from a layers immunoblot experiment, conducted as provided generally in U.S. Pat. Nos. 6,602,661, 6,969,615, 7,214,477 and 7,838,222; U.S. Publ. No. 20110306514; and in Chung & Hewitt, Meth Mol Biol, Prot Blotting Detect, Kurlen & Scofield, eds. 536:139-148, 2009. Hence, images of eight membranes are presented, where the eight membranes were stacked on a treated breast cancer tissue specimen, with the first membrane closest to the tissue section and the 8^{th} membrane being most distal from the tissue section. Each section was stained for total protein using labeled streptavidin following treatment of the transferred molecules with a commercially available biotinylation kit (ex, Pierce, #20217). That is reflected in the lower row of photographs that depict the degree of fluorescence for each membrane and it can be seen that the amount of protein diminished for the more superior filters. Each of the other membranes was treated with a specific commercially available antibody that binds a particular marker. That primary antibody can be labeled with a detectable marker or the primary antibody can be unlabeled and detected using a secondary labeled antibody that binds the first antibody if used. The detectable label generally is different from that used to assess to protein. For example, if fluorescence is used, the total protein can be detected with a fluorophore that yields a green color and the specific marker can be detected with a fluorophore that yields a red color. It can be seen that the levels of individual markers vary from filter to filter (the first filter is a control), from what would be considered minimal or no labeling in the second filter to high labeling in the 4^{th} and 6^{th} filters. The markers there were assessed, from 2nd to 8^{th} filter, were PTEN, pAKT (T308), pPDK1 (S241), HER4, MUC4, HER2 and vimentin. See, Example 6B

FIG. 3A shows a drawing of the VEGF signal transduction pathway representing multiple biomarkers in the pathway.

FIG. 3B shows a drawing of the PI3K/AKT/mTOR signal transduction pathway representing multiple biomarkers in the pathway.

FIG. 3C shows biological pathways targeted for therapy in renal cell carcinoma based on knowledge of the underlying genetic changes and downstream biological consequences (Vasudev et al. BMC Medicine 2012 10:112).

FIG. 4A shows a plot of responder and non-responder patients and the aggregate score for each retrospective patient sample generated from the assigned scores of five measured VEGF biomarkers (p-PRAS40, VEGFA, VEGFR1, VEGFR2 and PDGFRβ) in advanced renal cell carcinoma (RCC) FFPE tissue obtained prior to the administration of sunitinib. In this plot the predetermined cut off value or threshold value for predicting response to sunitinib was calculated to be 19, which corresponds to a sensitivity of 87.5% (correct responder prediction of 28 out of 32 samples and a specificity of 73.3% (correct non-responder prediction of 11 out of 15 samples) with an accuracy (overall percent correct) of 83%. This plot was derived from the data disclosed in Tables 4A and 5A which were obtained using the materials and method set forth in Example 2.

FIG. 4B shows a plot of responder and non-responder patients and the aggregate score for each retrospective patient sample generated from the assigned scores of three measured VEGF biomarkers (VEGFR1, VEGFR2 and VEGFA) in advanced renal cell carcinoma (RCC) FFPE tissue obtained prior to the administration of sunitinib. In this plot, the predetermined cut off value or threshold value for predicting response to sunitinib was calculated to be 24, which corresponds with a sensitivity of 81.8% (correct responder prediction of 27 out of 33 samples and a specificity of 83.3% (correct non-responder prediction of 15 out of 18 samples) with an accuracy (overall percent correct) of 82.3%. This plot was derived from the data disclosed in Tables 4C and 5C which were obtained using the materials and method set forth in Example 3.

FIG. 4C shows an example of images of two different kidney cancer samples, one sunitinib responder (top) and one non-responder (bottom), where a panel five VEGF biomarkers were measured using L-IHC methods as described above in FIG. 2. Intensity of labeled biomarkers appears brighter for several markers measured in the responder sample as compared to the non-responder sample. See Example 2.

FIG. 5A shows a plot of responder and non-responder patients and the aggregate score for each retrospective patient sample generated from the assigned scores of six measured mTOR biomarkers (mTOR, pmTOR (Ser 2448), p4EBP1 (Ser 65), p4EBP1 (Thr 37-46), PRAS40, pAKT (Substrate)) in advanced renal cell carcinoma (RCC) FFPE tissue obtained prior to the administration of an mTOR inhibitor (everolimus and/or temsirolimus). In this plot, the predetermined cut off value or threshold value for predicting response to an mTOR inhibitor (everolimus and/or temsirolimus) was calculated to be 10, which corresponds with a sensitivity of 58% (correct responder prediction of 7 out of 12 samples and a specificity of 81% (correct non-responder prediction of 17 out of 21 samples) with an accuracy (overall percent correct) of 73%. This plot was derived from the data disclosed in Table 6 which were obtained using the materials and method set forth in Example 4.

FIG. 5B shows a plot of responder and non-responder patients and the aggregate score for each retrospective patient sample generated from the assigned scores of three measured mTOR biomarkers (pmTOR (Ser 2448), p4EBP1 (Ser 65), p4EBP1 (Thr 37-46)) in advanced renal cell carcinoma (RCC) FFPE tissue obtained prior to the administration of an mTOR inhibitor (everolimus and/or temsirolimus). In this plot, the predetermined cut off value or threshold value for predicting response to an mTOR inhibitor (everolimus and/or temsirolimus) was calculated to be 6, which corresponds to a sensitivity of 67% (correct responder prediction of 8 out of 12 samples and a specificity of 81% (correct non-responder prediction of 17 out of 21 samples) with an accuracy of 76%. This plot was derived from the data disclosed in Table 6 which were obtained using the materials and method set forth in Example 5.

FIG. 5C shows an example of images of two different kidney cancer patients, one TORISEL® responder (top) and one non-responder (bottom), where a panel of six mTOR biomarkers were measured using L-IHC methods as described above in FIG. 2. Intensity of labeled biomarkers appears brighter for several markers measured in the responder sample as compared to the non-responder sample. See, Example 4.

FIG. 6A shows a plot of responder and non-responder patients and the aggregate score for each retrospective patient sample generated from the assigned scores of four measured mTOR biomarkers (pmTOR (Ser 2448), pERK, p4EBP1, HIF1a) in HER2 positive breast cancer FFPE tissue obtained prior to the administration of trastuzumab. In this plot, the predetermined cut off value or threshold value for predicting response to trastuzumab was calculated to be 6.5, which corresponds with a sensitivity of 88% (correct responder prediction of 28 out of 32 samples) and a specificity of 77% (correct non-responder prediction of 10 out of 13 samples) with an accuracy (overall percent correct) of 84%. This plot was derived from the data disclosed in Tables 7 and 8 which were obtained using the materials and method set forth in Example 6A.

FIG. 6B shows an example of two different breast cancer patients, one responder (top) and one non responder (bottom), where a panel of four mTOR biomarkers were measured using L-IHC methods as described above in FIG. 2. Intensity of labeled biomarkers appears brighter for several markers measured in the non-responders, suggesting that the mTOR pathway is activated, thereby conferring a resistance mechanism to HER2-inhibition. Annotated area in H&E-stained tissue section (Left) can be used for the orientation of corresponding regions of interest (ROI) in L-IHC layers. The samples chosen for illustration purposes in this figure show that several markers (e.g. two or more) are required to differentiate responder and non-responder patients. However, not all biomarkers of resistance are differentially expressed in each responder or non-responder. As can be seen in the four images from this particular responder one of the four resistance markers (HIF1α) is clearly expressed (far right). Only through using a combination of four different pathway proteins (and/or their phosphorylation status) was it possible to differentiate responders from non-responders. See, Example 6A

FIG. 6C shows the distribution of responders and non-responder patients and the combined expression levels of four mTOR biomarkers (pmTOR (Ser 2448), pERK, p4EBP1, HIF1a) in HER2 positive breast cancer FFPE tissue obtained prior to the administration of trastuzumab in a dot histogram with cut off value of 6.5 obtained by the receiver operating characteristic (ROC) curve analysis. See Example 6A

FIG. 6D shows the ROC curve that was calculated using the data from FIG. 6A with an area under the curve of 0.80 (95% confidence intervals of 0.6733 to 0.9637). A calculated cut off value to differentiate responders and non-responders to trastuzumab is 6.5. See Example 6A

FIG. 7 shows a plot of responder and non-responder patients and the aggregate score for each retrospective patient sample generated from the assigned scores of five measured VEGF biomarkers (VEGFR1, VEGFR2 and VEGFA) in advanced renal cell carcinoma (RCC) FFPE tissue obtained prior to the administration of sunitinib. In this plot the predetermined cut off value or threshold value for predicting response to sunitinib is represented as a range (gray area delineated with a dotted line). An aggregate score above the top dotted line corresponds to greater than 95.5% accuracy for predicting response to sunitinib; below the bottom dotted line corresponds to greater than 85.7% accuracy for predicting non-response to sunitinib (assuming the patient numbers in the gray box are not included in the calculation of accuracy, only those above and below the grey box). Aggregate scores that fall between the two dotted lines (gray box), are considered indeterminate with respect to prediction; patient aggregate scores that fall within the gray box would carry no prediction.

### DETAILED DESCRIPTION

### A) Introduction

The present disclosure relates to tests for predicting the responsiveness or non-responsiveness of a solid tumor to a therapeutic agent that inhibits, or impacts, activation of a signal transduction pathway. In general these tests utilize two or more biomarkers associated with that pathway which, in combination, aid in predicting therapeutic response. The activation of the signal transduction pathway is shown by measurement of protein expression levels in the signal transduction pathway, also referred to herein as “signaling effector proteins” or generally as “biomarkers”, that taken individually, collectively or in aggregate assess the likelihood a solid tumor will be responsive to a therapeutic agent. In certain embodiments, two or more signaling effector proteins are measured (qualitatively or quantitatively) in a sample obtained from a patient with a solid tumor. In order to maintain morphology and location of the biomarker, the samples can comprise solid tissue processed for protein detection, such as immunohistochemistry (IHC). In one aspect, the tumor and non-tumor cells are delineated, the biomarkers measured, a score or value assigned to each measured biomarker and the assigned scores combined to obtain an aggregate score. This aggregate score can also be referred to herein as a “predictive score”.

This predictive score, generated from a patient sample, provides meaningful data about the responsiveness or non-responsiveness of a pathway specific therapeutic agent when compared to a pre-determined cut off for predicting response, also referred to interchangeably herein as a “threshold value”. In certain aspects, this pre-determined cut off is calculated based on a data set generated from analysis of retrospective samples (e.g. samples collected before treatment, wherein clinical and pathology information was available after and/or during treatment). It is understood that the threshold value for predicting response is determined from the empirical data obtained from the retrospective samples and that a good fit of responders and non-responders is used to calculate the threshold value. In this retrospective study samples were obtained from patients diagnosed with a solid tumor (e.g. kidney, breast, lung, ovarian, pancreatic, etc.), but prior to treatment with a known signal transduction inhibitor (e.g. HER2, mTOR or VEGF inhibitors). Additional information was subsequently provided based on patient treatment, wherein the retrospective samples were classified (e.g., complete response, partial response, stable disease or non-response). A panel (e.g. two or more biomarkers) is measured in these retrospective samples and a value in the form of an assigned score is designated for each biomarker. See, Example 1 for a scoring method. This assigned score correlates to the inferred amount of protein measured in each sample. Each assigned score per sample is combined to obtain an aggregate score, which was compiled into a data set where a pre-determined cut off, either as a range or a single number, for predicting responsiveness or non-responsiveness for a therapeutic agent was calculated. See, Examples 2-6.

In certain embodiments the signal transduction pathway is the VEGF pathway. In particular are provided methods herein for predicting whether a patient with a solid tumor will respond to a therapeutic agent that inhibits a VEGF pathway. In certain other embodiments the signal transduction pathway is the PI3K/AKT/mTOR pathway, also referred to herein generally as the “mTOR” pathway. In particular are provided methods herein for predicting whether a patient with a solid tumor will respond to a therapeutic agent that inhibits an mTOR pathway.

In one embodiment, the solid tumor is renal cell carcinoma (RCC) and the expression levels of two or more proteins in the VEGF pathway are measured, the measurements combined and an aggregate score is obtained which is compared to a predetermined cut off for predicting responsiveness or non-responsiveness to a VEGF inhibitor on a RCC solid tumor. In another embodiment the solid tumor is renal cell carcinoma (RCC) and the expression level of two or more proteins in the mTOR pathway are measured, the measurements combined and an aggregate score is obtained which is compared to a predetermined cut off for predicting responsiveness or non-responsiveness to an mTOR inhibitor.

In other certain embodiments, disclosed herein are methods for assessing the likelihood and/or predicting if a HER2 positive solid tumor will respond to a HER2 inhibitor (e.g. a therapeutic agent that inhibits HER2 dimerization or the HER2 downstream pathway). In this instance, demonstration of the activation of the mTOR pathway, as determined by the present methods, based on a predictive score indicates the likelihood a HER2 positive solid tumor will not be responsive to the HER2 inhibitor as a single therapy. It is theorized that activation of mTOR acts as bypass or “short circuit” that obviates the effectiveness of blocking HER2 and its downstream mediators. In certain aspects, these HER2 positive tumors, where the mTOR pathway has been shown to be active with the present methods, can be responsive to an mTOR inhibitor either alone or in combination with a HER2 inhibitor. The present methods provide a means for identifying or selecting patients that while they have a HER2 positive solid tumor, would not likely be responsive to a HER2 inhibitor (e.g. HERCEPTIN) taken alone. In this way the present methods can be used to avoid unnecessary and expensive treatment.

The present methods provide valuable information (e.g. a predictive score), for an oncologist and ultimately the patient. This information can be in the form of a report, which can comprise a treatment recommendation based on the predictive score.

### B) Definitions

As used herein, the terms “a” or “an” are used, as is common in patent documents, to include one or more than one, independent of any other instances or usages of“at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term “aggregate score” refers to the combination of assigned scores from the measured biomarkers. In one embodiment the aggregate score is a summation of assigned scores. In another embodiment, combination of assigned scores involves performing mathematical operations on the assigned scores before combining them into an aggregate score. In certain, embodiments, the aggregate score is also referred to herein as the “predictive score”.

As used herein, the terms “assess”, “assessing”, and the like are understood broadly and include obtaining information, e.g., determining a value, whether through direct examination or by receiving information from another party that performs the examination.

As used herein, the term “assigned score” refers to the numerical value designated for each of the biomarkers or signaling effector proteins after being measured in a patient sample. The assigned score correlates to the absence, presence or inferred amount of presence of protein measured for each biomarker in the sample. The assigned score can be generated manually (e.g. by visual inspection) or with the aid of instrumentation for image acquisition and analysis. In certain embodiments, the assigned score is determined by a qualitative assessment, for example, fluorescence can be visually scored by a user on a graded scale of zero to three, with zero representing no label and four representing a large amount of label. In other aspects the graded scale can be zero to ten, zero to 12 or zero to 20, or some combination thereof. There is no intended limitation on the graded scale used to generate an assigned score for each measured biomarker. In further embodiments, the assigned score is a combination of the intensity of the labeled biomarker related to the area of label within a region of interest, such as when L-IHC methods are used. See, Example 1.

As used herein, the terms “biomarker”, “marker” (or fragment thereof) and their synonyms, which are used interchangeably, refer to molecules that can be evaluated in a sample and are associated with a physical condition. A biomarker comprises a characteristic that can be objectively measured and evaluated as an indicator of a normal biological process, a pathogenic process, or a pharmacologic response to a therapeutic intervention, for example. A biomarker can be used in many scientific fields, such as, in screening, diagnosis and patient monitoring. For example, a markers include expressed genes or their products (e.g. proteins) that can be detected from a human samples, such as blood, serum, solid tissue, and the like, that is associated with a physical or disease condition. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. Exemplary biomarkers can comprise a molecule, such as, a protein, a protein subunit, a mutant protein, or a mutation on a protein, a phosphoprotein and so on, that is detectable. The term “biomarker” can also refer to a portion of a polypeptide (parent) sequence that comprises at least 5 consecutive amino acid residues, at least 10 consecutive amino acid residues, at least 15 consecutive amino acid residues, and retains a biological activity and/or some functional characteristics of the parent polypeptide, e.g. antigenicity or structural domain characteristics. The present biomarkers refer to those tumor antigens present on or in cancerous cells or tumors and which are part of a signal transduction pathway. It is also understood in the present methods that use of the biomarkers in a panel can each contribute equally to the aggregate score or certain biomarkers can be weighted wherein the markers in a panel contribute a different weight or amount to the final aggregate score.

When applied to a protein or gene, e.g., mTOR, the term biomarker refers to the wild type protein or gene, as well as to naturally or artificially generated fragments, isoforms, splice variants, allelic variants, mutants, etc. One skilled in the art would appreciate that when the methods disclosed herein are applied to non-human mammals, appropriate orthologs of the human biomarkers disclosed in the instant application would be used.

As used herein, the terms “cancer” and “cancerous” refer to or describe the pathological condition in mammals that is typically characterized by unregulated cell growth. Examples of cancer include but are not limited to, lung cancer, breast cancer, colon cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

As used herein, the term “cell or tissue sample” refers to biological samples comprising cells, e.g., tumor cells, that are isolated from body samples, such as, but not limited to, smears, sputum, biopsies, secretions, cerebrospinal fluid, bile, blood, lymph fluid, urine and feces, or tissue which has been removed from organs, such as breast, lung, intestine, skin, cervix, prostate, and stomach. For example, a tissue samples can comprise a region of functionally related cells or adjacent cells.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples,

The above enumerated actions can be performed by a healthcare provider, healthcare benefits provider, or patient automatically using a computer-implemented method (e.g., via a web service or stand-alone computer system).

As used herein, the terms “differentially expressed gene,” “differential gene expression” and their synonyms, which are used interchangeably, are used in the broadest sense and refers to a gene and/or resulting protein whose expression is activated to a higher or lower level in a subject suffering from a disease, specifically cancer, such as lung cancer, relative to its expression in a normal or control subject. The terms also include genes whose expression is activated to a higher or lower level at different stages of the same disease. It is also understood that a differentially expressed gene can be either activated or inhibited at the nucleic acid level or protein level, or can be subject to alternative splicing to result in a different polypeptide product. Such differences can be evidenced by a change in mRNA levels, surface expression, secretion or other partitioning of a polypeptide, for example. Differential gene expression can include a comparison of expression between two or more genes or their gene products (e.g., proteins), or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, the term “down regulation” with respect to measured biomarkers, refers to a differential, decreased level of the biomarkers, e.g. by a differential expression of the genes, a decreased level of genes and gene products (e.g. proteins) or an increased level of activity. When down regulated, the level of the biomarker is measurably lower in a patient sample as compared to a reference sample.

As used herein, the term “effector protein” also referred to herein interchangeably as “signaling effector protein” refers to an intracellular protein (or a receptor or a ligand that when bound to a receptor activates a signal transduction cascade) that is a component of a signal transduction pathway and that can be chemically altered resulting in the acquisition or loss of an activity or property. In some embodiments, an “effector protein” is a “biomarker.” Such chemical alteration can include any of the post-translational modifications listed below as well as processing by proteinases. In one aspect, effector proteins are chemically modified by phosphorylation and acquire protein kinase activity as a result of such phosphorylation. In another aspect, effector proteins are chemically modified by phosphorylation and lose protein kinase activity as a result of such phosphorylation. In another aspect, effector proteins are chemically modified by phosphorylation and lose the ability to form stable complexes with particular proteins as a result of such phosphorylation. Exemplary effector proteins include, but are not limited to, mTOR proteins, VEGF proteins, TSC proteins, Akt proteins, Erk proteins, p38 proteins, and Jnk proteins. In regard to post-translational modifications of effector proteins, an effector protein can have one or more sites, referred to herein as a “post-translational modification site,” which are characteristic amino acids of the effector protein where a post-translational modification can be attached or removed in the course of a signal transduction event.

As used herein, the term “gene expression profiling” is used in the broadest sense, and includes methods of quantification of mRNA and/or protein levels in a biological sample.

As used herein, the term “healthcare provider” refers individuals or institutions which directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

In some aspects, a healthcare provider can administer or instruct another healthcare provider to administer a therapy comprising a therapeutic agent that inhibits a signal transduction pathway, e.g., the mTOR pathway or the VEGF pathway. A healthcare provider can implement or instruct another healthcare provider or patient to perform, e.g., the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapeutic agent (for example, a therapy comprising a therapeutic agent that inhibits a signal transduction pathway, e.g., the mTOR pathway or the VEGF pathway), commence the administration of a therapeutic agent, cease the administration of a therapeutic agent, continue the administration of a therapeutic agent, temporarily interrupt the administration of a therapeutic agent, increase the amount of administered therapeutic agent, decrease the amount of administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapeutic agent by at least another therapeutic agent, combine a therapeutic agent with at least another treatment or additional therapeutic agent.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare benefits provider can authorize or deny, for example, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration a therapeutic agent, commencement of the administration of a therapeutic agent, cessation of the administration of a therapeutic agent, continuation of the administration of a therapeutic agent, temporary interruption of the administration of a therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapeutic agent by at least another therapeutic agent, or combine a therapeutic agent with at least another treatment or additional therapeutic agent. In addition a healthcare benefits provides can, e.g., authorize or deny the prescription of a therapy, authorize or deny coverage for therapy, authorize or deny reimbursement for the cost of therapy, determine or deny eligibility for therapy, etc.

As used herein, the term “HER2 positive” refers to over expression of the HER2 protein, i.e. shows an abnormal level of expression in a cell from a disease within a specific tissue or organ of the patient relative to the level of expression in a normal cell from that tissue or organ. Patients having a cancer characterized by over expression of the HER2 receptor can be determined by standard assays known in the art. In certain embodiments, over expression is measured in fixed cells of frozen or paraffin-embedded tissue sections using immunohistochemical (IHC) detection. When coupled with histological staining, localization of the targeted protein can be determined and extent of its expression within a tumor can be measured both qualitatively and semi-quantitatively. Such IHC detection assays are known in the art and include the Clinical Trial Assay (CTA), the commercially available LabCorp® 4D5 test, and the commercially available DAKO HercepTest® (DAKO, Carpinteria, Calif.). The latter assay uses a specific range of 0 to 3+ cell staining (0 being normal expression, 3+ indicating the strongest positive expression) to identify cancers having over expression of the HER2 protein. Thus, patients having a cancer characterized by over expression of the HER2 protein in the range of 1+, 2+, or 3+, or in the range of 2+ or 3+, or 3+ are particularly encompassed.

As used herein, the term “HER2 pathway specific drug” referred to herein interchangeably with “HER2 inhibitor” and refers to molecules, such as proteins or small molecules that can significantly reduce HER2 properties (e.g., dimerization and signal transduction activation). Such HER2 inhibitors include anti-HER2 antibodies, e.g. trastuzumab, pertuzumab, or cetuximab. Trastuzumab (sold under the trade name HERCEPTIN®) is a recombinant humanized anti-HER2 monoclonal antibody used for the treatment of HER2 over-expressed/HER2 gene amplified metastatic breast cancer. Trastuzumab binds specifically to the same epitope of HER2 as the murine anti-HER2 antibody 4D5. Trastuzumab is a recombinant humanized version of the murine anti-HER2 antibody 4D5, referred to as rhuMAb 4D5 or trastuzumab) and has been clinically active in patients with HER2-overexpressing metastatic breast cancers that had received extensive prior anticancer therapy. Trastuzumab and its method of preparation are described in U.S. Pat. No. 5,821,337.

As used herein, the term “index score” refers to a value assigned to a biomarker, calculated from assessment of retrospective clinical. See, Table 9. In this instance, the mean scores for each marker were related to yield an index score, that is, the mean value for the non-responder group was divided by the mean value for the responder group to yield an index score. Alternatively, the index score can also be calculated by dividing the mean score for a particular biomarker from the responder group by the mean score for the corresponding biomarker in the non-responder group. It is understood that the values above and below this index score will be inverted. That index score can be used to obtain a threshold value for predicting responsiveness of the patient to one or more pathway-specific drugs.

As used herein, the term “inhibitor” refers to any molecule or other agent capable of inhibiting (e.g., partially or completely blocking, retarding, interfering with) one or more biological activities (e.g., a physiologically significant enzymatic activity) of a target molecule such as mTOR, HER2, VEGF, ANG2 etc. Examples include small molecules such as rapamycin and rapamycin analogs, antibodies, short interfering RNA (siRNA), short hairpin RNA (shRNA), antisense molecules, ribozymes, etc. An inhibitor may inhibit synthesis of a target polypeptide (e.g., by inhibiting synthesis of, or causing destabilization of, an mRNA that encodes the polypeptide, or by inhibiting translation of the polypeptide), may accelerate degradation of the polypeptide, may inhibit activation of the polypeptide (e.g., by inhibiting an activating modification such as phosphorylation or cleavage), may block an active site of the polypeptide, may cause a conformational change in the polypeptide that reduces its activity, may cause dissociation of an active complex containing the polypeptide, etc. An inhibitor may act directly by physical interaction with a target molecule, or indirectly, for example by interacting with a second molecule whose activity contributes to activation of the target molecule (e.g., a molecule that activates the target molecule, e.g., by phosphorylating it), by competing with the target molecule for binding to a substrate, activator, or binding partner needed for activity of the target molecule, etc. For example, mTOR inhibitors are molecules that inhibit activation of the mTOR complex, such as, mTORC1.

As used herein, the term “mTOR” refers to the mammalian target of rapamycin. mTOR is also known as a mechanistic target of rapamycin or FK506 binding protein 12-rapamycin associated protein 1 (FRAP1). Human mTOR is encoded by the FRAP1 gene. mTOR is a serine/threonine protein kinase that regulates cell growth, cell proliferation, cell motility, cell survival, protein synthesis and transcription. mTOR belongs to the phosphatidylinositol 3-kinase-related kinase protein family.

As used herein, the terms “mTOR pathway”, also referred to interchangeably herein as “PI3K/AKT/mTOR pathway”, refers to a signal transduction pathway comprising all molecules that interact directly or indirectly with mTOR, and thus are molecules upstream and downstream of mTOR, such as, mTORC1. For example, HER2 is known to activate PI3K and AKT. HER2 is a member of the EGFR family, which is known to activate the mTOR pathway. Hence, HER2 is an upstream member of the mTOR pathway. See, for example, Nahta et al., Clin Breast Cancer, Suppl. 3:572, 2010.

As used herein, the term “mTOR pathway specific drug” also referred to herein interchangeably as “mTOR inhibitor” refers to an inhibitor of the expression or activation, or both expression or activation, of a member of the mTOR pathway. For example, an mTOR pathway inhibitor can inhibit the expression or activation, or both, of AKT, mTOR, pTSC2, HIF1α, pS6, p4EBP1, PI3K, STAT3, as well as any receptor or receptor ligand that activates any component of the mTOR pathway. This list of members of the mTOR pathway is exemplary, and is not meant to be exhaustive.

As used herein, the term “normalization” and its derivatives, when used in conjunction with measurement of biomarkers across samples and time, refer to mathematical methods where the intention is that these normalized values allow the comparison of corresponding normalized values from different datasets in a way that eliminates or minimizes differences and gross influences.

As used herein, the terms “panel of markers”, “panel of biomarkers” and their synonyms, which are used interchangeably, refer to more than one marker that can be detected from a human sample that together, are associated with the presence of a particular cancer. In a particular embodiment of the present application, the presence of the biomarkers are not individually quantified as an inferred value to indicate the presence of a cancer, but the measured biomarkers are assigned a score and the assigned score (optionally normalized, transformed and/or weighed) is combined to provide an aggregate score. As disclosed above, each marker (optionally transformed) in the panel may be given the weight of 1, or some other value that is either a fraction of 1 or a multiple of 1, depending on the contribution of the marker to the signal transduction pathway of the solid tumor being assessed for drug responsiveness and the overall composition of the panel.

As used herein, the term “pathology” of (tumor) cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein the term “pathway-specific drug” refers to a drug designed to inhibit or block a signal transduction pathway by interacting with, or targeting, a component of the pathway to inhibit or block a protein-protein interaction, such as receptor dimerization, or to inhibit or block an enzymatic activity, such as a kinase activity or a phosphatase activity. Some targeted therapies block specific enzymes and growth factor receptors involved in cancer cell proliferation. These drugs are sometimes called signal transduction inhibitors.

Targeted cancer therapies have been developed that interfere with a variety of other cellular processes. FDA-approved drugs that target these processes are listed below.

As used herein, the term “predictive score” refers to a value or values calculated from measurement of the present biomarkers from a patient sample following biostatistical analysis. In certain embodiments the predictive score is the combination of the assigned scores for each biomarker measured in a sample, also referred to herein as an “aggregate score”. In other certain embodiments, the predictive score is calculated from a single measured biomarker and may be the assigned score, a ratio of the assigned score or some other value calculated based on the assigned score. In yet other certain embodiments, the predictive value may be a compilation or collection of values, also referred to herein as a predictive signature, of the measured biomarkers. In this instance, the individual predictive values comprising the signature may be the assigned score, a ratio of the assigned score or some other value calculated based on the assigned score.

As used herein, the term “response” or “responsiveness”, refers to a tumor response, e.g. in the sense of reduction of tumor size or inhibiting tumor growth. The term shall also refer to an improved prognosis, e.g. reflected by an increased time to recurrence, which is the period to first recurrence censoring for second primary cancer as a first event or death without evidence of recurrence, or an increased overall survival, which is the period from treatment to death from any cause. To, “respond,” or to have a, “response,” means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response, i.e., will exhibit a lack of response or be “non-responsive”.

A tumor is “sensitive” or “responsive” to a therapeutic agent if the agent inhibits (i.e., reduces) the growth rate of the tumor. Typically the growth rate of the tumor is detectably lower following exposure to the therapeutic agent and/or in the presence of the agent (e.g., after administration of the agent to a subject) than it was prior to the exposure and/or in the absence of the agent. In one aspect, the growth rate, e.g., cell proliferation rate, is decreased by at least a predetermined amount. For example, in certain embodiments a tumor is considered responsive to an agent if the proliferation rate following exposure to the agent is reduced by at least 10%, at least 20%, at least 30%, at least 40%, at least 50%, at least 60%, at least 70%, at least 80%, at least 90%, at least 100%, at least 150% (1.5 fold), at least 200% (2-fold), at least 3-fold, at least 5-fold, at least 10-fold, at least 20-fold, or more, relative to the growth rate prior to exposure to the agent. In some embodiments the proliferation rate is reduced to 0, or the number of cells decreases. For example, the number of cells may decline at a rate that is at least 10%, at least 20%, at least 30%, at least 40%, at least 50%, at least 60%, at least 70%, at least 80%, at least 90%, at least 100%, at least 150% (1.5 fold), at least 200% (2-fold), at least 3-fold, at least 5-fold, at least 10-fold, or at least 20-fold, as great as the proliferation rate prior to exposure to the agent. A predetermined amount may be any other value that falls within any sub-range, and has any specific value (specified to the tenths place), within the limits of the values set forth above.

It will be appreciated that the exposure can be a single exposure or can be ongoing exposure, e.g., as when a patient is administered a course of a chemotherapeutic agent that includes administration of multiple doses over a period of time. Growth typically refers to cell proliferation. In the case of a tumor, cell proliferation typically results in an increase in volume of the tumor. A tumor that is sensitive or responsive to a therapeutic agent is said to “respond” to the agent.

A tumor or tumor cell line that is not sensitive to a therapeutic agent is said to be “resistant” or “non-responsive” to the agent.

As used herein, the terms “sample” or “tissue sample” or “patient sample” or “patient cell or tissue sample” or “specimen” each refers to a collection of similar cells obtained from a tissue of a subject or patient. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; or cells from any time in gestation or development of the subject. The tissue sample may contain compounds which are not naturally intermixed with the tissue in nature such as preservatives, anticoagulants, buffers, fixatives, nutrients, antibiotics, or the like. In one aspect of the invention, tissue samples or patient samples are fixed, particularly conventional formalin-fixed paraffin-embedded samples. Such samples are typically used in an assay for receptor complexes in the form of thin sections, e.g. 3-10 μm thick, of fixed tissue mounted on a microscope slide, or equivalent surface. Such samples also typically undergo a conventional re-hydration procedure, and optionally, an antigen retrieval procedure as a part of, or preliminary to, assay measurements.

As used herein, the terms “signaling pathway” or “signal transduction pathway” refers to a series of molecular events usually beginning with the interaction of cell surface receptor and/or receptor dimer with an extracellular ligand or with the binding of an intracellular molecule to a phosphorylated site of a cell surface receptor. Such beginning event then triggers a series of further molecular interactions or events, wherein the series of such events or interactions results in a regulation of gene expression, for example, by regulation of transcription in the nucleus of a cell, or by regulation of the processing or translation of mRNA transcripts. In one aspect, signaling pathway means either the Ras-Raf-MAPKinase pathway, the PI3K-Akt pathway, the VEGF pathway, the HER2 pathway or an mTOR pathway. The “Ras-MAPK pathway” refers to a signaling pathway that includes the phosphorylation of a MAPK protein subsequent to the formation of a Ras-GTP complex. The “PI3K-Akt pathway” refers to a signaling pathway that includes the phosphorylation of an Akt protein by a PI3K protein. The “mTOR pathway” refers to a signaling pathway comprising one or more of the following entities; an mTOR protein, a PI3K protein, an Akt protein, an S6K1 protein, an FKBP protein, including an FKBP12 protein, a TSC1 protein, a TSC2 protein, a p70S6K protein, a raptor protein, a rheb protein, a PDK protein, a 4E-BP protein, wherein each of the proteins may be phosphorylated at a post-translational modification site. mTOR pathways may also include the following complexes: FKBP12//mTOR, raptor//mTOR, raptor//4E-BP1, raptor//S6K1, raptor//4E-BP1//mTOR, raptor//S6K1//mTOR. The proteins of the preceding two sentences are well known to those of skill in the art and are described in the following references, which are incorporated by reference: Sawyers, Cancer Cell, 4: 343-348 (2003); Xu et al, International J. Oncol., 24: 893-900 (2004); Fong et al, Proc. Natl. Acad. Sci., 100: 14253-14258 (2003); Fruman et al, Eur. J. Immunol., 25: 563-571 (1995); Hidalgo et al, Oncogene, 19: 6680-6686 (2000); and the like.

As used herein, the term “subject” refers to an animal, such as a mammal, including a human or non-human animal for which diagnosis, prognosis, or therapy is desired. The term “nonhuman animal” includes all vertebrates, e.g., mammals and non-mammals, such as nonhuman primates, sheep, dogs, cats, horses, cows, bears, chickens, amphibians, reptiles, etc. The terms “patient” and “human subject” may be used interchangeably herein.

As used herein, the terms “treat” or “treatment” refer to both therapeutic treatment and prophylactic or preventative measures, wherein the object is to prevent or slow down (lessen) an undesired physiological change or disorder, such as the progression of a disease or condition. Beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented. Accordingly, terms such as “treating” or “treatment” or “to treat” refer to both (1) therapeutic measures that cure, slow down, and lessen symptoms of, and/or halt progression of a diagnosed pathologic condition or disorder and (2) prophylactic or preventative measures that prevent and/or slow the development of a targeted pathologic condition or disorder. Consequently, those in need of treatment include those already with the disorder; those prone to have the disorder, and those in whom the disorder is to be prevented.

In order to treat a patient, samples from the patient can be obtained before or after the administration of a therapy comprising a therapeutic agent that inhibits a signal transduction pathway, e.g., the mTOR pathway or the VEGF pathway. In some cases, successive samples can be obtained from the patient after treatment has commenced or after treatment has ceased. Samples can, e.g., be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to yet another healthcare provider, healthcare benefits provider or the patient. Similarly, the measuring/determination/calculation of assigned scores, measuring/determination/calculation of predictive scores, measurement/determination/calculation of predetermined cut off values, comparisons between predictive scores and predetermined cut off values, evaluation of the comparisons between predictive scores and predetermined cut off values, and treatment decisions can be performed by one or more healthcare providers, healthcare benefits providers, and/or clinical laboratories.

As used herein, the term “tumor” refers to an abnormal mass of tissue that results from unregulated excessive cell division. A tumor can be benign (not cancerous) or malignant (cancerous). “Tumor” includes disorders characterized by unregulated excessive division of cells derived from the organ of origin. Such disorders include malignant hematolymphatic disorders such as leukemia, lymphoma, myeloma, and myeloproliferative disorders as well as solid tumors that comprise the other cancer types especially epithelial- and soft tissue-derived cancers including the carcinomas and sarcomas, respectively. Tumors are diagnosed histologically or cytologically (e.g., performed on a cell or tissue sample) and extent (stage) of cancer can be determined using any of a variety of art-accepted methods including physical diagnosis, imaging studies, biochemical tests, etc. Specific, non-limiting examples of tumors include sarcomas, prostate cancer, breast cancer, endometrial cancer, hematologic tumors (e.g., leukemia, Hodgkin's and non-Hodgkin's lymphoma, multiple myeloma and other plasma cell disorders, myeloproliferative disorders), brain tumors (e.g., low grade astrocytoma, anaplastic astrocytoma, glioblastoma multiforme, oligodendroglioma, and ependymoma), and gastrointestinal stromal tumors (GIST). Sarcomas include osteosarcoma, Ewing's sarcoma, soft tissue sarcoma, and leiomyosarcoma. Additional examples of malignant tumors include small cell and non-small cell lung cancer, kidney cancer (e.g., renal cell carcinoma), hepatocellular carcinoma, pancreatic cancer, esophageal cancer, colon cancer, rectal cancer, stomach cancer, breast cancer, ovarian cancer, bladder cancer, testicular cancer, thyroid cancer, head and neck cancer, thyroid cancer, etc.

As used herein, the term “up-regulation” with respect to measured biomarkers, refers to a differential, increased level of the biomarkers, e.g. by a differential expression of the genes, an increased level of genes and gene products (e.g. proteins) or an increased level of activity. When up-regulated, the level of the biomarker is significantly higher in a patient sample as compared to a reference sample.

As used herein, the term “VEGF,” refers to a molecule which stimulates, induces, activates or results in angiogenesis. Vascular endothelial growth factor (VEGF) engages a cell surface receptor (VEGFR), a tyrosine kinase, which when activated, that is, binds VEGF, triggers a signaling cascade resulting in, for example, vascularization, angiogenesis and so on.

As used herein, the term “VEGF pathway specific drug” also used herein interchangeably as “VEGF inhibitor” or “VEGF pathway inhibitor” refers to an inhibitor of the expression or activation, or both expression or activation, of a member of the VEGF pathway. For example, a VEGF pathway inhibitor can inhibit the expression or activation, or both, of VEGFA, VEGFR1, VEGFR2, VEGFB, HIF1α, HIF1β, HIF2α, PDGFRα or PDGFRβ, as well as any receptor or receptor ligand that activates any component of the VEGF pathway. This list of member of the VEGF pathway is exemplary, and is not meant to be exhaustive.

As used herein, the term “VEGF pathway,” refers to a signal transduction pathway comprising molecules found on and in a cell that have a role in the effects noted from VEGF engaging the receptor thereof. Thus, the molecules that are members of the VEGF pathway are those that mediate the signaling cascade that begins with VEGF engaging the VEGFR and ending with a cell activity that is triggered or halted by the VEGF-VEGFR interaction. For the purposes of the present methods, any molecule that is a biomarker for cancer that is in some way associated with VEGF and angiogenesis is contemplated to be considered part of a VEGF pathway.

### C) Development of Predictive Tests

Applicants herein disclose a model for developing and validating predictive tests for a range of disease types and targeted therapies. This model generally includes the following steps: 1) Selection of a targeted therapy for which a predictive test is desired; 2) Selection of candidate biomarkers; 3) Procurement of disease tissue samples from responders and non-responders; 4) Measurement of the candidate biomarkers in disease tissue samples; 5) Data analysis and selection of an optimum panel; 6) Development of a predictive algorithm based on the predictive biomarkers and retrospective samples (e.g. responders and non-responders to the selected target therapy); and 7) Transformation of the measured biomarker panel into a predictive score. This last step is performed with patient samples to generate a predictive score, also referred to herein as an aggregate score, to help select the optimum targeted therapy for the patient.

For ease of understanding the invention, each of the above steps will be described in detail followed by methods for predicting responsiveness or non-responsiveness of a solid tumor to a therapeutic agent including clinical application.

1) Identification of a Targeted Therapy for which a Predictive Test is Desired

The present invention is beneficial for targeted therapies that have been shown to benefit only a subset of patients to whom the drug is administered, such as 50% or fewer of patients. As will become clear from the following disclosure, targeted therapy, as used herein refers to a drug that inhibits or disrupts, either directly or indirectly, a signal transduction pathway. Targeted drugs or therapies are known generally for cancer indications, inflammatory indications, autoimmune indications, gastrointestinal indications, infectious disease indications, and so on. There is no intended limitation on the targeted drug that may be selected for testing to predict its effectiveness on the indication or disease. While targeted therapies have been developed to ameliorate a specific indication, in part because the target (e.g. agonist or antagonist) is present or up-regulated in the disease it is also well understood that for many indications only a certain percentage of any patient population will respond to the targeted therapy, either initially or over time (due to acquired resistant). This may be due to any number of factors and the non-responsiveness or resistance of the target therapy may be present initially (poor patient selection) or the resistance may be acquired (e.g. down-regulation of the target or activation of alternative disease pathways). The present predictive model and methods find use in patient selection for a targeted therapy and also for patient monitoring so that a treating physician may make decisions on when to change a targeted therapy or to better understand when an adjuvant therapy may be beneficial due to an activation, or de-activation, of a specific signal transduction pathway.

In this way, any targeted therapy, from any disease indication area, may be selected for which a predictive test is desired, provided that there is good understanding of the signal transduction pathway impacted by the targeted therapy and there is a nexus between the signal transduction and the disease. The latter should be implicit in the development of a targeted therapy depending on the mechanism of action. The signal transduction pathway and selection of corresponding biomarkers are described in more detail below.

One such disease indication area that is of particular interest is oncology. There are many oncology targeted therapies on the marker (drug product) and more in clinical development (drug candidate). See, Table 1 below for a partial list of oncology targeted therapies.

TABLE 1
Targeted Drugs for Cancer Indications

| | | | |
|---|---|---|---|
| | | Process | |
| Drug | Target | Targeted | Indication |

Monoclonal Antibodies

| | | | |
|---|---|---|---|
| Bevacizumab | Vascular | Angiogenesis | Colorectal |
| (AVASTIN) | endothelial | | (metastatic) |
| | growth factor | | |
| Cetuximab | Epidermal | Growth factor | Colorectal |
| (ERBITUX) | growth factor | signaling | |
| Trastuzumab | HER2 receptor | Growth factor | Breast |
| (HERCEPTIN) | | signaling | |

Small molecule inhibitors

| | | | |
|---|---|---|---|
| Imatinib | Bcr-Abl fusion | Growth factor | Chronic myeloid |
| (GLEEVEC/ | protein: Kit | signaling | leukemia, |
| GLIVEC) | | | Gastorintestinal |
| | | | stromal |
| Erlotinib | Epidermal | Growth factor | Non-small |
| (TARCEVA) | growth factor | signaling | cell lung |
| Sunitinib | Receptor | Cellular | Renal cell |
| (SUTENT) | tyrosine kinase | signaling | carcinoma, |
| | | | gastrointestinal |
| | | | stromal |
| Everolimus | Mammalian target | mTOR pathway | Renal cell |
| (AFINITOR) | of rapamycin | | carcinoma, breast |
| Temsirolimus | Mammalian target | mTOR pathway | Renal cell |
| (TORISEL) | of rapamycin | | carcinoma |

In certain embodiments, the target is the PI3K/AKT/mTOR signal transduction pathway. There are nearly a dozen different therapeutic agents designed to target the mTOR pathway that are either on the market or are in late stage clinical testing. The drugs or drug candidates are being used or tested against numerous tumor types including, lymphomas, kidney cancers or breast cancers. A partial list of mTOR inhibitors and their indications is set forth in following Table 2.

TABLE 2
mTOR Targeted Drugs

| | Tumor Indications |
|---|---|
| mTOR targeted therapy | (Approved or in late |
| Generic name (BRAND NAME) | stage clinical testing) |
| Temsirolimus (TORISEL) | Kidney, Breast |
| Everolimus (AFINITOR) (RAD001) | Kidney, Breast, Brain (SEGA) |
| | Pancreatic (PNET) |
| Ridaforolimus (Taltorvic) | Sarcomas (bone and soft tissue) |
| Serolimus (RAPAMUNE) | Solid tumors |
| AZD8055 | Lymphoma/Brain (Gliomas) |

In other certain embodiments, the target is the VEGF signal transduction pathway. There are also nearly a dozen different therapeutic agents designed to target the VEGF pathway that are either on the market or are in late stage clinical testing. The drugs or drug candidates are being used or tested against numerous tumor types including, colon cancers, kidney cancers or breast cancers. A partial list of VEGF inhibitors and their indications is set forth in following Table 3

TABLE 3
VEGF Pathway Targeted Drugs

| Drug name | Type | Mechanism of Action | Clinical Stage |
|---|---|---|---|
| Bevacizumab | Humanized | Blocks VEGF-A | Approved for metastatic |
| (Avastin) | monoclonal | binding to receptor | CRC, NSCLC, RCC; |
| | antibody | | recurrent GBM |
| Sunitinib | Small molecular | Inhibits signaling of | Approved for metastatic |
| (Sutent) | RTK inhibitor | VEGFRs, PDGFR's, | RCC, imatinib resistant |
| | | FLT-3, CSF1R | GIST, PNET |
| Sorafenib | Small molecular | Inhibits signaling of | Approved for metastatic |
| (Nexavar) | RTK inhibitor | VEGFRs, Raf, | RCC, HPCC |
| | | PDGFR's, KIT | |
| Pazopanib | Small molecular | Inhibits signaling of | Approved for metastatic RCC |
| (Votrient) | RTK inhibitor | VEGFRs, PDGFR's, KIT | |
| Vandetanib | Small molecular | Inhibits signaling of | Aprroved for metastatic |
| (Caprelsa) | RTK inhibitor | VEGFRs, PDGFR's, | medullary thyroid cancer |
| | | EGFR | |
| Axitinib | Small molecular | Inhibits signaling of | Approved for RCC |
| (Inlyta) | RTK inhibitor | VEGFRs, PDGFR's, KIT | that failed first-line therapy |
| Aflibercept | Chimeric soluble | Binds VEGFA, | Phase 3 multiple tumor types |
| (Zaltrap) | receptor | VEGFB, and PIGF | |
| AMG386 | Peptidobody | Binds Angiopoietin-1 and -2 | Phase 3 multiple tumor types |
| Motesanib | Small molecular | Inhibits signaling of | Phase 3 multiple tumor types |
| | RTK inhibitor | VEGFRs, PDGFR's, KIT | |
| Cediranib | Small molecular | Inhibits signaling of | Phase 3 multiple tumor types |
| (Recentin) | RTK inhibitor | VEGFRs, PDGFR's, KIT | |
| Cabozantinib | Small molecular | Inhibits signaling of | Phase 3 multiple tumor types |
| | RTK inhibitor | VEGFRs, PDGFR's, | |
| | | cMET, RET, KIT | |
| Tivozanib | Small molecular | Inhibits signaling of | Phase 3 metastatic RCC |
| | RTK inhibitor | VEGFRs, PDGFR's, KIT | |
| Regorafenib | Small molecular | Inhibits signaling of | Phase 3 relapsed CRC |
| | RTK inhibitor | VEGFRs, Raf, | and other tumors |
| | | PDGFR's, KIT | |
| Ramucirumab | Human | Blocks VEGFR2 | Phase 3 multiple tumor types |
| | monoclonal | signaling | |
| | antibody | | |

### 2) Selection of Candidate Biomarkers

The known mechanism of action of the drug for which a predictive test is desired is the starting point for selecting a pool of biomarkers to be tested. For example, if the drug is designed to target the mTOR pathway, the biomarkers may include, but not be limited to, those listed in Table 6 and Example 4. If the drug is designed to target the VEGF pathway, the biomarkers may include, but not be limited to, those listed in Table 4 and Example 2. In certain embodiments, the expression level of biomarkers (e.g., proteins), in both their activated (e.g., phosphorylated) and inactivated states are included as part of the candidate pool since nucleic acid testing fails to account for posttranslational modifications and phosphorylation levels, e.g. Example 4, mTOR and pmTOR. In addition, other biomarkers from additional pathways, which may be interconnected, may also be included in the candidate pool of biomarkers that are screened. In further embodiments, the candidate pool of biomarkers may differ depending on the disease tissue, even when the same signal transduction pathway is being assessed and the same targeted drug is being tested for responsiveness to the disease.

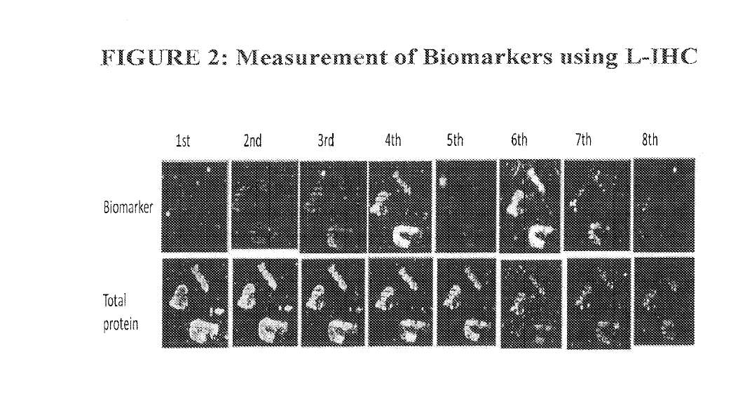

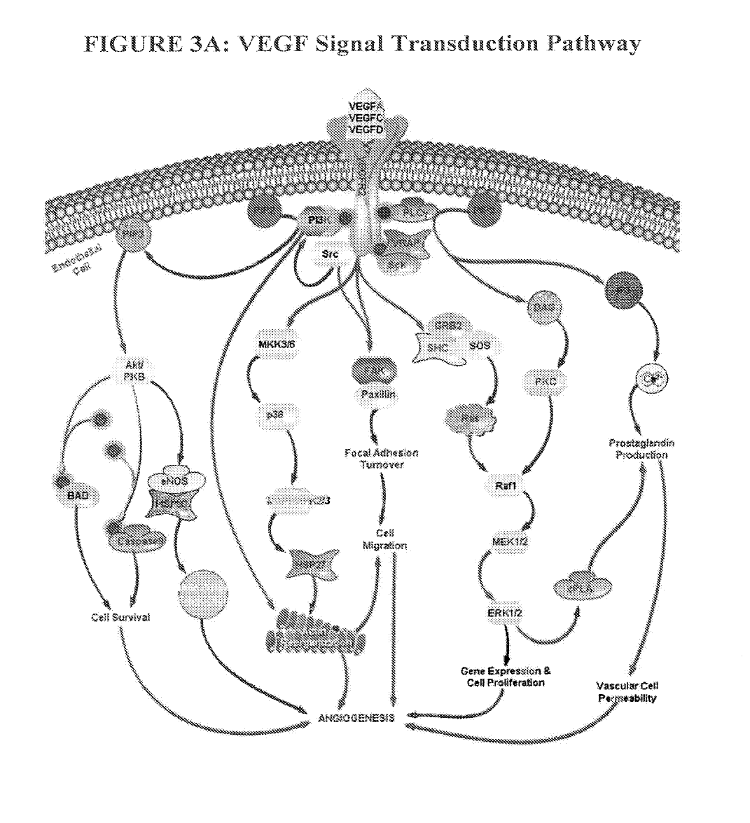

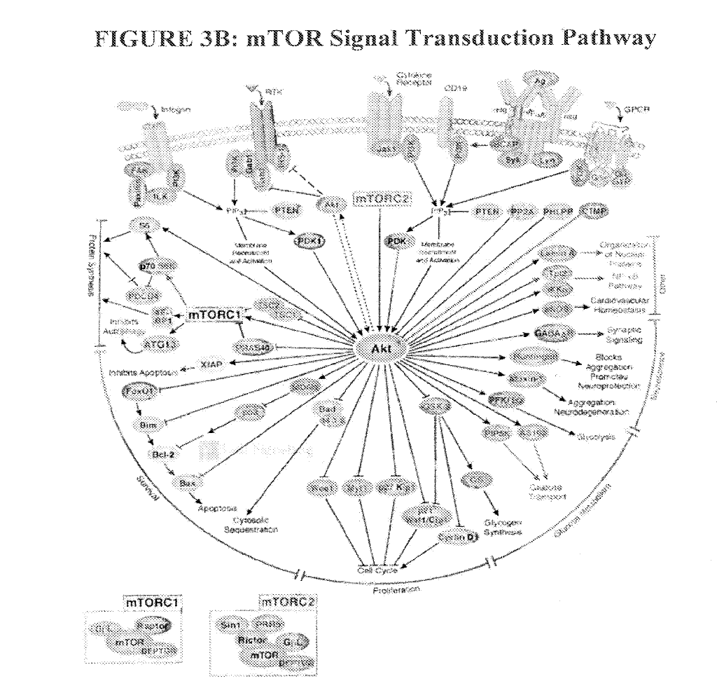

If a multiplex technology is employed such as those described herein as many as 15-25 or more candidate proteins may be surveyed from small amounts of tissue. Many of the known signal transduction pathways are well mapped (See, FIG. 3) and reagents for assessing the effector proteins in the pathway are generally available from commercial sources. Alternatively, reagents can be made by one of skill in art using well know techniques.

The signal transduction pathway includes any pathway involved in growth (e.g. proliferation or angiogenesis) or maintenance (e.g. enzyme metabolism) of a solid tumor. It is understood that the signal transduction pathways are broad and often interconnected and as such the nomenclature for referring to such a pathway may be by the receptor (e.g. EGFR), the drug target (mTOR), or the ligand or factor (e.g. TGF-beta). The drug target may be the receptor or the ligand, or any other protein in the cascade that if inhibited or blocked would lead to disruption of the signal transduction pathway. There is no intended limitation on the signal transduction pathway in the present methods and such pathways include, but are not limited to, PI3K/AKT/mTOR, HER2, HER3, VEGF, HIF, Ang-2, EGFR, PDGF, PDGFR, EGF, TGF-β, FGF, FGFR, NGF, TGF-α, IGF-I, IGF-II, and IGFR. Signal transduction pathways may also be generally referred to as cytokine pathways, receptor tryrosine kinase (RKT) pathways, MAPK pathways, etc.

One of skill in art, reviewing the scientific literature, would be able to select a sufficient number of biomarkers for a candidate pool. Applicants herein performed this analysis for both the mTOR signal transduction pathway and the VEGF pathway to obtain a candidate pool of biomarkers. See, Example 2-6. As described in further detail below, the candidate pool is then measured in retrospective samples in order to identify biomarkers that either individually or collectively are predictive for response of the disease tissue or tumor to a targeted therapy. While the targeted signal transduction pathway is used as a road map for selecting the candidate pool of biomarkers, it is contemplated that only a subset (e.g. 5%-75%) of the candidate biomarkers tested will ultimately become part of the final predictive panel. For example as shown in Example 2 and 3, while two different final predictive panels included five (5) and three (3) biomarkers respectively, the initial candidate pool included fifty five biomarkers.

It is further contemplated that a candidate pool of biomarkers for a pathway (e.g. mTOR) would be selected, but that ultimately the final predictive panel selected would be disease (e.g. kidney and breast cancer) and/or targeted therapy specific. See Examples 4 and 6. The present biomarkers are well known, and the sequence of which can be found in data bases such as GenBank.

In certain embodiments, candidate mTOR pathway biomarkers include, but are not limited to, any protein in FIG. 3B. In other embodiments, candidate mTOR pathway biomarkers include, but are not limited to, ras, p110, p85, p13K, PTEN, Akt, PDK1, mTOR, Rictor, Raptor, IRS1, PIP2, PIP3, Proctor, mLST8, PLD1, PA, Redd1/2, FKBP12, TSC1, FKBP38, FK506, FK520, ERK, RSK1, LKB1, Sin1, AMPK, TSC1, Rheb, PRAS40, PHLPP1/2, GSK3b, PKA, 4EBP1, eiF4E, eiF4A, FOXO1, Rag A/B/C/D, SHIP1, pAKT Substrate, TSC2, p70S6K, ATG13, 4E-BP1, PGC-1, S6K, Tel2, BRAF, PPAR, AMPK, Dv1, HIF1α, NF1, ROC1, eIF4B, S6, eEF2K, PDCD4, various GPCR's, HIF1α, STK11, p53, SGK, PKC, TORK3, FKBP and so on including phosphorylated versions of these proteins, see, for example, Hernandez-Aya et al., The Oncologist, 16:404-414, 2011; Darwish et al., J. Urology, 2013 (published online November 2012); Borders et al., Am J Health Syst Pharm, 67(24):2095-2106 (2010); WO 2007/047754. Any molecule involved in the metabolism, that is activation or inactivation, of the mTOR pathway is contemplated in the practice of the present methods.

In certain embodiments, Applicants herein selected mTOR, p-mTOR (Ser 2448), pPTEN, AKT, pAKT (ser 473), pAKT (Thr 308), PI3K, 4EBP1, p4EBP1 (Thr 37/46), HIF1α, Vimentin, HER2, HER4, MUC4, PDK, pPDK (Ser 241), ERK, pERK (Thr 202/Tyr 204), Actin as mTOR pathway biomarkers for the candidate pool for screening in HER2 positive breast cancer retrospective samples. In other embodiments, Applicants selected mTOR, p-mTOR (Ser 2448), p-mTOR (Ser 2481), AKT, pAKT (ser 473), pAKT (substrate), PI3K, TSC1, pTSC (Thr 1462), TSC2, pTSC2 (Ser 939), PRAS40, pPRAS40 (Thr 246), pPRAS40 (Ser 183), 4EBP1, p4EBP1 (Ser 65), p4EBP1 (Thr 3746), Rictor, pRictor (Thr 1135), HIF1α, HIF1β, HIF2α, VEGFA, VEGFR1, VEGFR2, pVEGFR2 (Tyr 996), pVEGFR2 (Tyr1175), VEGFB, PDGFRα, PDGFRβ, CAIX, CD31, CD34, EGFR, Integrin αV, Integrin α6, FAK, PIGF, Vimentin, ERK, pERK, Raf-B, Raf-1, Raptor, S6 Ribosomal protein, pS6 Ribosomal protein (Ser235/236), p70 S6 Kinase, p70 S6 Kinase, (Thr389), p70 S6 Kinase (Ser371), VHL (von Hippel-Lindau), pEGFR (Tyr 845), pHER2 (Tyr1248)/EGFR (Tyr1173), pHER2 (Tyr 1248), pHER2 (Tyr 1221/1222), pFAK (Tyr 397) mTOR pathway biomarkers for the candidate pool for screening in renal cell carcinoma. This candidate pool, one for screening activation of the mTOR pathway in HER2 positive breast cancer and the other for screening activation of the mTOR pathway in renal cell carcinoma, resulted in two predictive biomarker panels for screening the effectiveness of an mTOR inhibitor on these two patient populations. The selection of the mTOR predictive biomarkers, in combination, is disclosed in detail below.

In certain embodiments, candidate VEGF pathway biomarkers include, but are not limited to any protein in FIG. 3A. In other embodiments, candidate VEGF pathway biomarkers include, but are not limited to, pi3K, Akt, mTOR (and those entities of the mTOR pathway), PIP2, PIP3, ras, PLCγ, VRAP, Sck, Src, BAD, eNOS, HSP90, Caspase9, MKK3/6, p38, MAPKAPK2/3, HSP27, Cdc42, FAK, Paxillin, GRB2, SHC, SOS, DAG, PKC, SPK, Raf1, MEK1/2, ERK1/2, IP3, CALN, NFAT, cPLA, COX2, VEGFA, VEGFR1, VEGFR2, VEGFB, HIF1α, HIF1β, HIF2α, PDGFRα, PDGFRβ and so on, see, for example, Hicklin et al., J. Clin Oncol., 23:1011-1027, 2005.

Applicants herein selected mTOR, p-mTOR (Ser 2448), p-mTOR (Ser 2481), AKT, pAKT (ser 473), pAKT (substrate), PI3K, TSC1, pTSC (Thr 1462), TSC2, pTSC2 (Ser 939), PRAS40, pPRAS40 (Thr 246), pPRAS40 (Ser 183), 4EBP1, p4EBP1 (Ser 65), p4EBP1 (Thr 3746), Rictor, pRictor (Thr 1135), HIF1α, HIF1β, HIF2α, VEGFA, VEGFR1, VEGFR2, pVEGFR2 (Tyr 996), pVEGFR2 (Tyr1175), VEGFB, PDGFRα, PDGFRβ, CAIX, CD31, CD34, EGFR, Integrin αV, Integrin α6, FAK, PIGF, Vimentin, ERK, pERK, Raf-B, Raf-1, Raptor, S6 Ribosomal protein, pS6 Ribosomal protein (Ser235/236), p70 S6 Kinase, p70 S6 Kinase, (Thr389), p70 S6 Kinase (Ser371), VHL (von Hippel-Lindau), pEGFR (Tyr 845), pHER2 (Tyr1248)/EGFR (Tyr1173), pHER2 (Tyr 1248), pHER2 (Tyr 1221/1222), pFAK (Tyr 397) as a candidate pool to screen for up-regulation of proteins in the VEGF pathway in renal cell carcinoma retrospective samples.

Once a sufficient number of biomarkers have been selected (e.g. 5-20) they are measured in retrospective samples obtained from patients treated with the target therapy. The samples were collected before the patients were treated; outcome data provided on responsiveness was reviewed after the candidate biomarkers were measured in the respective samples to generate a training set.

3) Procurement of Disease Tissue from Responders and Non-Responders

In order to ascertain which, if any, of the candidate biomarkers help predict response to the drug of interest it is necessary to procure representative tumor samples of patients to whom the drug has been administered. Importantly, accompanying these samples must be reliable outcome data detailing the patient's response to the drug. This should include information regarding patient's prognosis at onset of therapy, changes in tumor size during treatment, duration of treatment, progression free survival and overall survival.

A key advantage of the methods disclosed herein is that they can be used for drugs that have completed clinical trials and regulatory approval and are used in clinical practice. For such drugs it is preferable to obtain tumor samples from multiple, distinct medical centers so as to eliminate the potential of bias that might accompany samples procured from a single site. These biases might include tissue handling factors that influence the quality of protein in tissue such as delay of fixation time, time of fixation, and tissue processing conditions.

In certain embodiments, sufficient numbers of tumor samples to develop and validate the aggregate score are obtained. As a rule of thumb when using statistical models to determine relative weights of biomarkers, and to have a fully validated, marketable diagnostic test acceptable to most of the medical community, regulators, and many healthcare payers in most of the world, one should have at least 10 samples for each biomarker being considered in the statistical model and at least 10 samples for any combination of two or more biomarkers in that model. In logistic regression models, the rate of events per variable should be at least 10 (Peduzzi P, Concato J, Kemper E, Holford T R, Feinstein A R. A simulation study of the number of events per variable in logistic regression analysis. J Clin Epidemiol. 1996 December; 49(12):1373-9). For example, if weights are being developed for four biomarkers at least 40 samples from responders and at least 40 samples from non-responders are needed; if weights will also include a weight for the product of two of these biomarkers an additional 10 samples from each of responders and non-responders are needed. When examining sensitivity and specificity using a threshold value for an aggregate score that has already been developed, at least 20 samples for each of responders and non-responders are required. This sample size ensures that the expected width of the 95% confidence interval for either sensitivity or specificity includes no more than half of the range between 0% and 100% no matter what values of sensitivity or specificity are observed. Once one can set minimum thresholds for acceptable sensitivity and specificity, estimates of sensitivity and specificity can be used in formal calculations to determine the required numbers of samples from responders and non-responders to have 80% statistical power to reject sensitivity and specificity below those minimum thresholds.

Example 2 details the procurement of retrospective samples from patients treated with a VEGF inhibitor (SUTENT). Example 4 details the procurement of retrospective samples from patients treated with an mTOR inhibitor (Everolimus or Temsirolimus). Example 6 details the procurement of retrospective samples from patients treated with a HER2 inhibitor (HERCEPTIN).

### 4) Measurement of Biomarkers in Tissue Samples

There are many methods known in the art for measuring either gene expression (e.g. mRNA) or the resulting gene products (e.g. polypeptides or proteins) that can be used in the present methods.

The method of measuring signaling effector proteins is not necessarily limited to any one assay format or platform. For example, the presence and quantification of one or more antigens or proteins in a test sample can be determined using one or more immunoassays that are known in the art. Immunoassays typically comprise: (a) providing an antibody that specifically binds to the biomarker (namely, an antigen or a protein); (b) contacting a test sample with the antibody; and (c) detecting the presence of a complex of the antibody bound to the antigen in the test sample.

Well known immunological binding assays include, for example, an enzyme linked immunosorbent assay (ELISA), which is also known as a “sandwich assay”, an enzyme immunoassay (EIA), a radioimmunoassay (RIA), a fluoroimmunoassay (FIA), a chemiluminescent immunoassay (CLIA) a counting immunoassay (CIA), a filter media enzyme immunoassay (MEIA), a fluorescence-linked immunosorbent assay (FLISA), agglutination immunoassays and multiplex fluorescent immunoassays (such as the Luminex Lab MAP), immunohistochemistry (IHC), etc. For a review of the general immunoassays, see also, Methods in Cell Biology: Antibodies in Cell Biology, volume 37 (Asai, ed. 1993); Basic and Clinical Immunology (Daniel P. Stites; 1991).

In standard IHC in which one biomarker is analyzed per tissue section, there may not be sufficient tissue present in serial sections of tissue to analyze 5-10 biomarkers especially in core needle biopsies that have scant numbers of cancer cells.

The immunoassay can be used to determine a test amount of an antigen in a sample from a subject. First, a test amount of an antigen in a sample can be detected using the immunoassay methods described above. If an antigen is present in the sample, it will form an antibody-antigen complex with an antibody that specifically binds the antigen under suitable incubation conditions described above. The antibody-antigen complex is visualized, and subsequently measured, using reporter molecules directly or indirectly attached to the antibody. Suitable reporter molecules include fluorophores, including Quantum dots (Qdots), chromophores, chemiluminescent molecules, etc. and other labels well known to one of skill in the art. The amount of an antibody-antigen complex can be determined by comparing the measured value to a standard or control. The AUC for the antigen can then be calculated using techniques known, such as, but not limited to, a ROC analysis.

### Multiplex Tissue Analysis

Methods utilizing IHC can provide additional information (e.g. morphology, location of biomarkers) which can be important when analyzing biomarkers in a solid tumor. Such methods included layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 20110306514 (incorporated herein by reference); and in Chung & Hewitt, Meth Mol Biol., Prot Blotting Detect, Kurlen & Scofield, eds. 536:139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, Md.).

In lieu of L-IHC, other multiplex tissue analysis techniques might also be useful for identifying optimal biomarkers according to the present invention. Such techniques should permit at least five, or at least ten or more biomarkers to be measured from a single FFPE section due to the frequent scarcity of pre-treatment samples (especially needle biopsies). Furthermore, for reasons stated above, it is advantageous for the technique to preserve the localization of the biomarker and be capable of measuring the activation (e.g., phosphorylation) of various signaling effector proteins (e.g. mTOR pathway proteins).

The L-IHC method can be performed on any of a variety of tissue samples, whether fresh or preserved. For example, in the studies exemplified below, kidney and breast cancer assays were performed on samples from pathology tissue archives received after IRB approval of the protocol. The samples were coded and included 33 formalin-fixed, paraffin-embedded (FFPE) kidney cancer tissue specimens from patients prior to treatment with everolimus and/or temsirolimus (See, Example 4 and 5), 33 FFPE breast cancer tissue specimens from patients prior to treatment with HERCEPTIN® (See, Example 6) and 48 FFPE kidney cancer tissue specimens from patients prior to treatment with SUTENT® (See, Example 2 and 3). The patients had been subsequently treated per standard of medical care and response to therapy is linked to the samples and includes non-response, stable disease, partial response and complete response based on the criteria used by the institution. The samples included core needle biopsies and surgical resections that were routinely fixed in 10% normal buffered formalin and processed in the pathology department. Standard five μm thick tissue sections were cut from the tissue blocks onto charged slides that were used for L-IHC. Expression of multiple biomarkers can be correlated with response to therapy.

Thus, L-IHC enables testing of multiple markers in a tissue section by obtaining copies of molecules transferred from this tissue section to plural bioaffinity-coated membranes in register to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10 μm thick coated polymer backbone with 0.4 μm diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, Md.). Thus, each membrane comprises a copy of the tissue and can be probed for a different biomarker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In other embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, Md.). That technique is described in U.S. Pat. No. 7,473,532.

Another is the multiplex IHC technique developed by GE Global Research (Niskayuna, N.Y.). That technique is described in U.S. Pub. Nos. 2008/0118916 and 2008/0118934. There, sequential analysis is performed on biological samples containing multiple targets including the steps of binding a fluorescent probe to the sample followed by signal detection, then inactivation of the probe followed by binding probe to another target, detection and inactivation, and continuing this process until all targets have been detected.

Another system that might be employed is the AQUA software system available from HistoRx (Branford, Conn.).

In other embodiments, multiplex tissue imaging can be performed when using fluorescence (e.g. fluorophore or Quantum dots) where the signal can be measured with the multispectral imagine system Nuance™ (Cambridge Research & Instrumentation, Woburn Mass.). As another example, fluorescence can be measured with the spectral imaging system SpectrView™ (Applied Spectral Imaging, Vista, Calif.). Multispectral imaging is a technique in which spectroscopic information at each pixel of an image is gathered and the resulting data analyzed with spectral image-processing software. For example, the Nuance system can take a series of images at different wavelengths that are electronically and continuously selectable and then utilized with an analysis program designed for handling such data. The Nuance system is able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio.

Another system that may be used includes reverse phase protein microarrays (RPMA), which are designed for quantitative, multiplexed analysis of proteins, and their posttranslational modified forms, from a limited amount of sample (Chiechi et al. Biotechniques 2012 September; PCT Publication No. WO 2007/047754).

In such multiplex assays, any of a number of different reporters can be used, such as, fluorescence molecules, chemiluminescence molecules, colloidal particles, such as, those carrying a metal, such as, gold, quantum dots (see, for example, US Publ. No. 2001/0023078, and U.S. Pat. Nos. 6,322,901 and 7,682,789), enzymes, which will require a substrate that on reaction yields a detectable signal, and so on, as a design choice, and as known in the art.

### Image Analysis

In the case of IHC or L-IHC, using, for example, fluorescent reporters and dyes, automated detection systems can be used to digitize images, to facilitate the process and which can enable a quantitative metric for analysis and comparison. There are several pathology imaging devices on the market including the BioImagene iScan Coreo system and the widely-used Aperio Scanscope system that can produce digital images of H&E stained as well as fluorescently-labeled slides. Other scanners include the 3D Histech Pannoramic SCAN system that images fluorescently-labeled slide, and the Dako ACIS system for brightfield imaging of slides. Fluorescently-labeled membranes can be scanned on the Typhoon Trio Plus system and image analysis performed using the Autoquant software. The Olympus VS110 Scanning system using OlyVIA software produces digital images of H&E-stained tissue and is best suited for producing digital fluorescent images from membranes. Image analysis can then performed using the Visiomorph image analysis software available from Visiopharm (Denmark). In this manner, scoring fluorescent signals may be performed visually using a 0, 1, 2 and 3+; or a 0, 1, 2, 3 and 4+ intensity scoring system either to determine the overall intensity corresponding to the cancer regions of interest or alternatively to obtain the product in which the factors include the percentage distribution of signal over ROIs and the signal intensity. Greater objectivity and continuous scale biomarker measurement can be obtained using image analysis software in the scoring scheme.

### 5) Data Analysis and Selection of an Optimum Panel

In identifying predictive biomarkers from the candidate pool a number of steps are performed in the analysis to select the optimum panel of biomarkers. These steps include 1) scoring the measured biomarkers to obtain an assigned score for each biomarker in a sample; 2) optionally performing an operation (e.g. transformation, weighting) on the assigned score and 3) combining the assigned scores to obtain an aggregate score. Applicants herein used the present scoring methods and analysis disclosed herein to select biomarkers that in combination were predictive for tumor response to a targeted therapy. This same scoring method, described in more detail below, may also be used when measuring two or more biomarkers in a patient sample. See, Clinical Use section below.

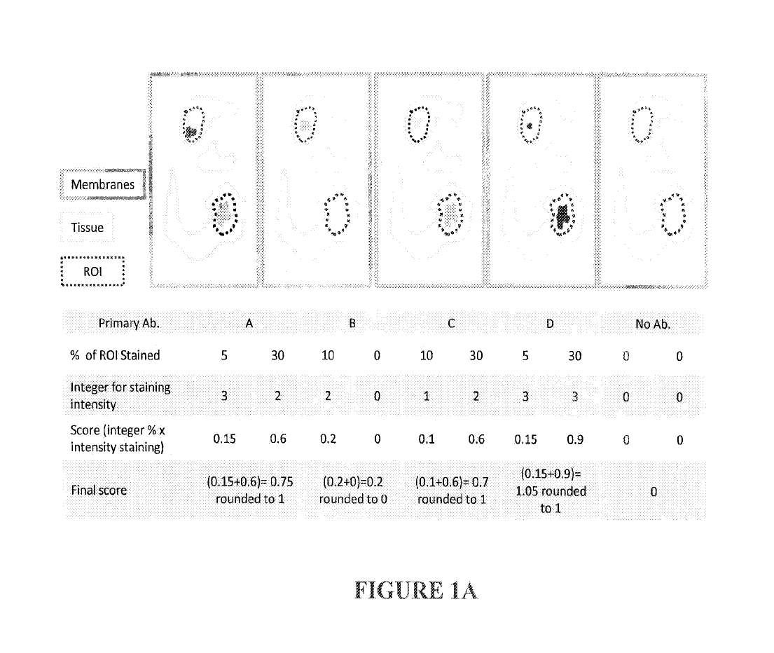

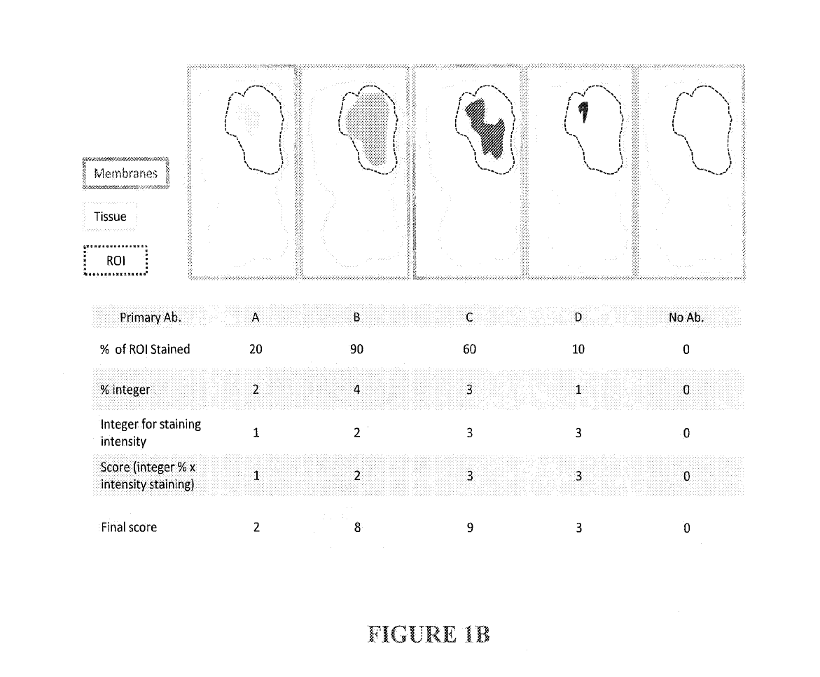

In certain embodiments the measured biomarkers are individually assigned a score following measurement wherein the assigned score is based on a graded scale and the value assigned (e.g. zero to four) is designated for each biomarker measurement based on an inferred and/or relative amount of biomarker measured in the sample. See FIG. 1 and Example 1 for exemplary assigned scoring methods.

In certain embodiments, the graded scale comprises zero to four; zero to 10; zero to 12; zero to 20; or some combination thereof. In an alternative embodiment the scale starts with 1 and not zero, either way, the smallest integer designates the absence of a biomarker (as evidenced by a lack of a signal in the methods used to measure the biomarker) and the largest number designates a high for the measured biomarker.

As described above, the biomarkers are measured by methods well known in the art, including acquisition of an image such as with IHC. In exemplary embodiments, L-IHC methods are used to label and measure multiple biomarkers, wherein one biomarker is labeled per membrane. The measured biomarkers are scored, wherein each biomarker is designated with an assigned value. These assigned scores are based on a graded scale, which may range from zero to a higher integer designated by the user that satisfactorily segregates the measured biomarkers and is amenable to further analysis and/or biostatics. It is understood that there are many different methodologies for scoring measured biomarkers and the user and/or pathologist may devise any scoring method that satisfactorily assigns a score based on an inferred amount of measured biomarker in the patient sample comprising cancerous cells. Herein, Applicants disclose two embodiments of scoring methods (See, FIG. 1 and Example 1) which were used to score each candidate biomarker.

In certain embodiments, the measured biomarkers are scored using a method that takes into account both the intensity of the labeled biomarker and the region of interest (ROI) area with labeled biomarker. The ROI is the cancerous cells that are delineated from non-cancerous or normal tissue. When using L-IHC methods, the ROI designation is transferred to each membrane in the stack. In one embodiment, intensity of signal for each measured biomarker is expressed as integers (e.g., 0, 1, 2, 3, 4) and multiplied by the fraction of the respective ROI area with labeled biomarker (e.g. 0%-100%) at the same intensity to obtain an assigned score. If more than one ROI with labeled biomarker is present on the L-IHC membrane the resulting numbers (e,g, 0.15+0.6) are summed (0.75) and rounded to the nearest integer (1) to obtain the overall assigned score for the biomarker on the membrane. See, Example 1A and FIG. 1A.

In another embodiment, the measured biomarkers are scored wherein the ROI area with labeled biomarker is designated as a graded scale (e.g., one to four) rather than a percentage and multiplied by the intensity of the labeled biomarker. In this instance, the intensity for each measured biomarker is expressed as an integer (e.g. 0, 1, 2, 3) and multiplied by percentage of ROI area labeled with biomarker expressed as an integer (e.g., 1, 2, 3, 4) to obtain an assigned score expressed as an integer (e.g., 0 to 12). If needed, the assigned score from each ROI on the same membrane are averaged (e.g., 6+8/2=7) to obtain an overall assigned score for the biomarker on the membrane expressed as an integer (e.g., 0 to 12). See, Example 1B and FIGS. 1B and 1C.

In further embodiments, following obtaining an assigned score an operation is performed on the assigned score before it is combined to obtain an aggregate score. In certain embodiments, the assigned score is reversed. In one embodiment, one or more of the measured biomarkers in a panel is designated with a reversed assigned score. In this instance, each biomarker is measured and an assigned score designated for each labeled biomarker. For example, if a graded scale of zero to 12 were being used the measured biomarkers would be assigned a score from zero to 12 based on the present scoring methods. One or more of those assigned scores (e.g. a biomarker with an assigned score of 3) would be subtracted from the total possible (e.g. 12) and designated with a reversed assigned score (e.g. 9). The compilation of assigned scores and reversed assigned scores would then be combined to obtain an aggregate score. This allows for using biomarkers in a signal transduction pathway that may actually be down-regulated when the pathway is activated, in combination with biomarkers that are up-regulated, but however are useful in demonstrating activation of the pathway.

In certain embodiments, mathematical operations (also referred to as transformations) may be performed on the assigned scores for one or more of the measured biomarkers before determining relative weights in the aggregate score. In certain embodiments, one or more of the measured biomarkers may be down-regulated when the pathway is activated, such that reversing its value as described above or reversing its mathematical sign facilitates combination with biomarkers that are up-regulated. In certain embodiments, one or more of the measured biomarkers may be mathematically centered to facilitate fitting of the statistical model that will be used to assign relative weights. For example, if biomarkers are scored from 0 through 12, the value 6 may be subtracted from each. In certain embodiments, assigned scores for one or more of the measured biomarkers may take a limited number of ordered values (e.g., 0, 1, 2, 3, 4) that do not have the interval property (e.g., the distances between each value do not necessarily reflect equal differences in biomarker expression) or the ratio property (e.g., the biomarker expression is twice as much in a sample with a score of 2 as it is in a sample with a score of 1). In these and other situations, the assigned scores for one or more of the measured biomarkers may be expanded into a group of associated scores, sometimes referred to as indicator variables or dummy variables, each of which will be assigned a weight when being combined into the aggregate score. In certain embodiments, the variance in the assigned score for one or more of the measured biomarkers may depend on the value of the assigned score. In these and other situations, the assigned scores for one or more of the measured biomarkers may be transformed using a one-to-one operation to stabilize the variance, for example but not limited to, base-10 logarithm, natural logarithm, square root, inverse (also called reciprocal), square, raising to a power other than half (i.e., square root) or two (i.e., square), and taking the are sine of values standardized to lie between −1 and 1. Transformations including but not limited to those just listed may also be used for other purposes, including but not limited to, minimizing the impact of extreme observations on determining relative weights and ensuring additivity of effects in the statistical model used to generate relative weights. In certain embodiments, the effects of two or more biomarkers are not adequately captured by an additive model, and this may remain true after transformation and application of relative weights. In these and other situations, the product of the possibly transformed assigned scores for two or more of the biomarkers may be generated and combined with individual assigned scores and any transformed assigned scores.

In other certain embodiments, the assigned scores are weighted. The choice of the biomarkers may be based on the understanding that each biomarker contributed equally to predicting the responsiveness or non-responsiveness for a therapeutic agent on a particular solid tumor. Thus in certain embodiments, the biomarker in the panel is measured and assigned a score wherein none of the biomarkers are given any specific weight. In this instance each marker has a weight of 1.

In other embodiments, the choice of the biomarkers may be based on the understanding that each marker, when measured and assigned a score, contributed unequally to predicting the responsiveness or non-responsiveness of a therapeutic agent on a particular solid tumor. In this instance, a particular biomarker in the panel can either be weighted as a fraction of 1 (for example if the relative contribution is low), a multiple of 1 (for example if the relative contribution is high) or as 1 (for example when the relative contribution is neutral compared to the other biomarkers in the panel). Thus, in certain embodiments, the present methods further comprising weighting the assigned score prior to obtaining an aggregate score by combining the assigned scores.

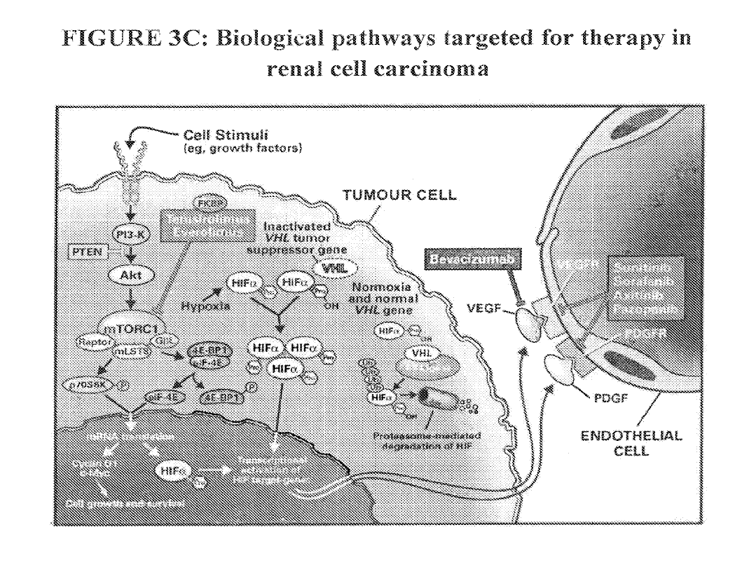

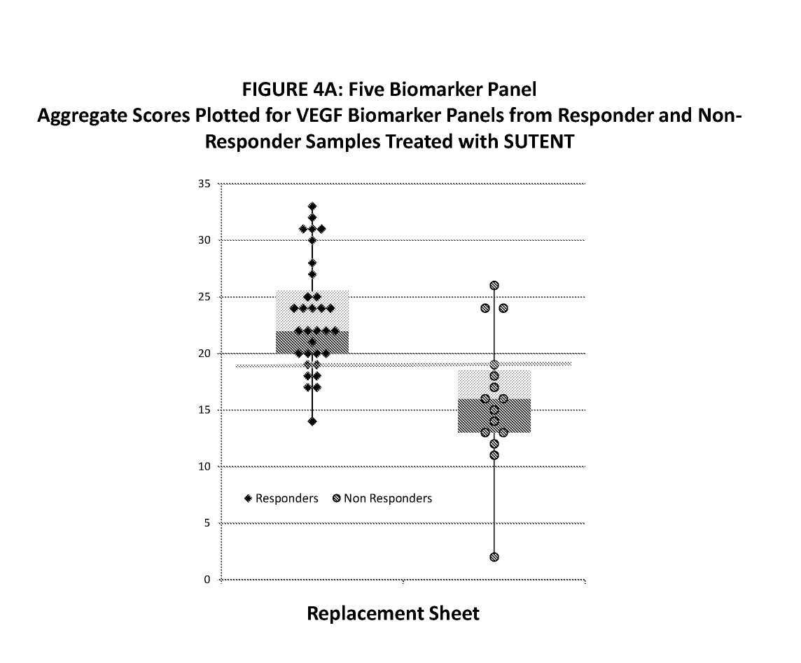

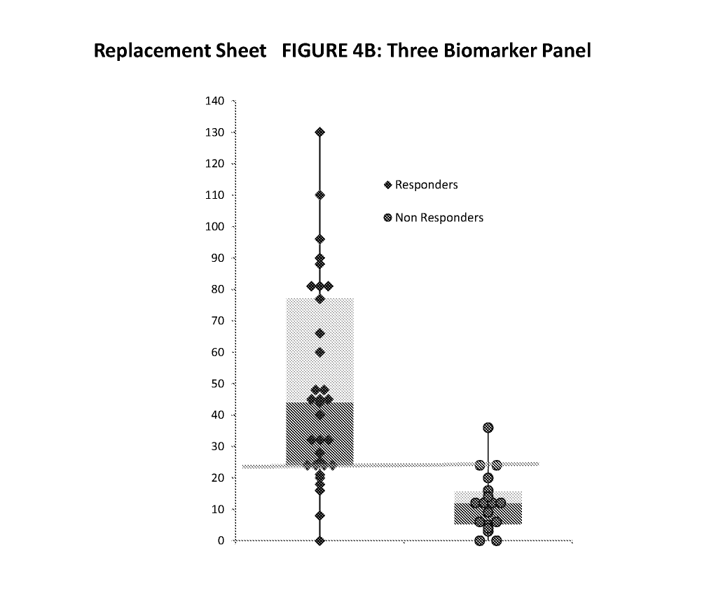

Following obtaining an assigned score for each candidate biomarker, and optionally performing on operation on the assigned score, the assigned score were evaluated. Based on the outcome data from the retrospective samples, biomarkers were selected that appeared to contribute to predicting responsiveness to the targeted therapy. See, Table 4. In general, the average assigned scores for each biomarker were calculated for responders and non-responders. The biomarkers whose average scores were not significantly different between responders and non-responders were in general dropped from the candidate pool of biomarkers. Generally, those with an assigned score that was differentiated across the two groups (responder and non-responders, across multiple samples, were selected for inclusion in the analysis to determine a predictive biomarker panel set. Thus, individual biomarkers were selected based on their assigned score, however it was the combination of these assigned scores to obtain an aggregate score that was predictive. It is contemplated that using the present scoring methods may identify individual biomarkers that may be predictive, however Applications found that combining the assigned scores could predict responsiveness of the disease tissue with greater than 80% accuracy. See, FIG. 4A. Using the present scoring and combination of assigned scores Applicants were able to select optimum biomarker panels for the two signal transduction pathways that were evaluated (VEGF and mTOR) in two different disease tissues (breast and kidney tumor) based on the present methodology. While only these two pathways were specifically evaluated, the present methods teach identifying a predictive biomarker panel and accompanying predictive algorithm for multiple signal transduction pathways and targeted therapies. mTOR and VEGF are only two exemplified pathways as are the targeted drugs evaluated with these pathways. New drug product and drug candidates are continually being developed that would benefit from the present model and tests.

In certain embodiments, the assigned scores are combined by summing the assigned scores to obtain an aggregate score. See Example 2 and Table 4A and 5A. In certain other embodiments, the aggregate score is obtained by summing all but one of the assigned scores and then multiplying this number by the remaining assigned score. See, Example 3 and Table 4C and 5C.

In certain embodiments, the relative weights for each biomarker may be determined using a likelihood ratio approach (Baker S G. Identifying combinations of cancer markers for further study as triggers of early intervention. Biometrics. 2000 December, 56(4):1082-7; Baker S G. The central role of receiver operating characteristic (ROC) curves in evaluating tests for the early detection of cancer. J Natl Cancer Inst. 2003 Apr. 2, 95(7):511-5; Eguchi S, Copas J. A class of logistic-type discriminant functions. Biometrika (2002) 89(1): 1-22; Pepe M S, Cai T, Longton G. Combining predictors for classification using the area under the receiver operating characteristic curve. Biometrics. 2006 March; 62(1):221-9) applied to assigned scores, each of which may have been transformed as described above, and which may be combined with each other as described above. This approach generates an ROC curve that lies above the ROC curve for other combinations (Zou K H, Liu A, Bandos A I, Ohno-Machado L, Rockette H E. Statistical Evaluation of Diagnostic Performance: Topics in ROC Analysis. CRC Press, Florida, 2012), thereby maximizing area under the ROC curve, also called AUC. In certain embodiments, the relative weights for each biomarker may be determined to provide an optimal linear combination using generalized linear models (McIntosh M W, Pepe M S. Combining several screening tests: optimality of the risk score. Biometrics. 2002 September; 58(3):657-64; Pepe et al 2006 Supra) fitted from assigned scores, each of which may have been transformed as described above, and which may be combined with each other as described above. Generalized linear models include but are not limited to logistic regression and probit regression. In certain embodiments, relative weights may be determined to meet optimality criteria other than generating an ROC curve that lies above the ROC curve for other combinations. These criteria include but are not limited to maximizing AUC, maximizing the area under the ROC curve to the left of some predetermined false positive rate (1-specificity) or above some predetermined sensitivity (partial AUC), maximizing sensitivity at some predetermined value of specificity, maximizing specificity at some predetermined value of sensitivity, maximizing the sum of sensitivity and specificity (equivalently, maximizing Youden's index, which is one less than the sum of sensitivity and specificity), and maximizing weighted sums of sensitivity and specificity. Linear combinations of biomarkers that maximize AUC may be obtained through likelihood-based approaches (Su J Q, Liu J S. Linear Combinations of Multiple Diagnostic Markers. Journal of the American Statistical Association 1993; 88:1350-1355; Liu A, Schisterman E F, Zhu Y. On linear combinations of biomarkers to improve diagnostic accuracy. Stat Med. 2005 Jan. 15; 24(1):37-47) or through distribution-free approaches (Pepe M S, Thompson M L. Combining diagnostic test results to increase accuracy. Biostatistics. 2000 June; 1(2):123-40; Pepe et al 2006 Supra, Vexler A, Liu A, Schisterman E F. Efficient design and analysis of biospecimens with measurements subject to detection limit. Biom J. 2006 August; 48(5):780-91; Ma S, Huang J. Regularized ROC method for disease classification and biomarker selection with microarray data. Bioinformatics. 2005 Dec. 15; 21(24):4356-62; Ma S, Huang J. Combining multiple markers for classification using ROC. Biometrics. 2007 September; 63(3):751-7).

In certain embodiments, biomarkers may be combined using other approaches. These approaches included but are not limited to model-free approaches (Pfeiffer R M, Bur E. A model free approach to combining biomarkers. Biom J. 2008 August; 50(4):558-70), Bayesian approaches (O'Malley A J, Zou K H. Bayesian multivariate hierarchical transformation models for ROC analysis. Stat Med. 2006 Feb. 15; 25(3):459-79. PMID: 16217836), discrimination rules, and classification and regression trees (Simon R. Roadmap for developing and validating therapeutically relevant genomic classifiers. J Clin Oncol. 2005 Oct. 10; 23(29):7332-41).

In certain embodiments, the relative weights used in the aggregate score will be used to determine more than one threshold. For example, thresholds may be provided such that one is expected to provide 80% sensitivity and the other is expected to provide 80% specificity. In these situations, aggregate scores falling between the thresholds may be reported and those reports may include estimates of, for example, the estimated probability of response with an associated 95% confidence interval.

Below follows disclosure and exemplary embodiments of optimum or predictive biomarker panels from the candidate pool of biomarkers that were identified by the Applicants using the present scoring and analysis methods.

### VEGF Pathway Biomarkers

The VEGF pathway comprises a number of molecular entities that interact in a sequential fashion to provide a signaling cascade or transduction mechanism or means that begins with a stimulus, such as, VEGF binding a VEGFR and culminating in a response of cell to that stimulus, such as, resulting in an observable tissue manifestation, such as, angiogenesis. Hence, a molecule triggers or induces a change, such as, phosphorylation of a first target molecule, such as, a VEGFR. Then the VEGFR acts on a second target molecule, for example, phosphorylating the second target molecule, which when phosphorylated is enabled to trigger or to induce a change in a third target molecule, and so on. Lists of proteins that are involved in the VEGF pathway can be found in commercial distributors of individual pathway components or of antibodies that bind individual pathway components, such as, Cell Signaling Technology, Inc. Danvers, Mass.; BioCarta LLC, San Diego, Calif.; and SABiosciences/Qiagen, Valencia, Calif., and include pI3K, Akt, mTOR (and those entities of the mTOR pathway), PIP2, PIP3, ras, PLCγ, VRAP, Sck, Src, BAD, eNOS, HSP90, Caspase9, MKK3/6, p38, MAPKAPK2/3, HSP27, Cdc42, FAK, Paxillin, GRB2, SHC, SOS, DAG, PKC, SPK, Raf1, MEK1/2, ERK1/2, IP3, CALN, NFAT, cPLA, COX2, VEGFA, VEGFR1, VEGFR2, VEGFB, HIF1α, HIF1β, HIF2α, PDGFRα, PDGFRβ and so on, see, for example, Hicklin et al., J. Clin Oncol, 23:1011-1027, 2005.

When a panel is used, that panel can comprise two or more, three or more, four or more, five or more, or more biomarkers, where the biomarkers comprise molecules of the VEGF pathway. However, the panel is not limited to only biomarkers of the VEGF pathway but can include other biomarkers known or found to be associated with a particular cancer or biomarkers from other interconnected signal transduction pathways.

### Kidney Cancer Biomarkers

In certain embodiments, biomarkers for assessing responsiveness of VEGF inhibitors screened in retrospective tissue samples from patients diagnosed with advanced renal cell carcinoma. See, Examples 2 and 3.

In one embodiment, the present methods predict responsiveness or non-responsiveness of a VEGF inhibitor on a RCC solid tumor by measuring VEGF pathway effector signaling proteins, also referred to herein as VEGF biomarkers. In one embodiment, the biomarkers demonstrating activation of the VEGF pathway in kidney cancer type solid tumors (e.g. renal cell carcinoma) may comprise any protein directly or indirectly involved with the activation of the VEGF pathway. In a particular embodiment, the present methods utilize biomarkers comprising VEGFA, VEGFR1, VEGFR2, p-PRAS40, VEGFB, HIF1α, HIF1β, HIF2α, PDGFRα or PDGFRβ for demonstrating activation and/or upregulation of the VEGF pathway in RCC solid tumors. The biomarkers may comprise two or more of any protein involved in signaling of the VEGF pathway, including two or more of the above listed biomarkers. In another embodiment, the biomarkers may comprise three or more, four or more or five or more, six or more of any protein involved in the signaling of the VEGF pathway, including three or more of the above listed biomarkers.

Reagents for detecting same are commercially available, such as, antibodies thereto, as well as secondary antibodies to serve a reporter function, second antibody to amplify signal, such as biotinylated second antibodies, and so on. For example, antibodies to the above are available commercially, such as, antibodies to VEGFA, VEGFR1, VEGFR2, VEGFB, PDGFRα and PDGFRβ are available from Santa Cruz Biotechnology, Inc. (Santa Cruz, Calif.) and to HIF1α, HIF1β and HIF2α are available from Abcam Inc. (Cambridge, Mass.) and p-PRAS40 from Cell Signaling Technology (Danvers, Mass.).

In a particular embodiment, the VEGF biomarkers may comprise VEGFA, VEGFR1, VEGFR2, and PDGFRβ. In another particular embodiment, the panel of VEGF biomarkers (effector signaling proteins) measured in a patient sample with a RCC solid tumor are VEGFA, VEGFR1, VEGFR2, and PDGFRβ.

In another particular embodiment, the VEGF biomarkers may comprise p-PRAS40, VEGFA, VEGFR1, VEGFR2 and PDGFRβ. In a particular embodiment, the panel of VEGF biomarkers (effector signaling proteins) measured in a patient sample with a RCC solid tumor are p-PRAS40, VEGFA, VEGFR1, VEGFR2 and PDGFRβ. See, FIGS. 4A and 4C; Example 2.

In yet another particular embodiment, the VEGF biomarkers may comprise VEGFR1, VEGFR2 and VEGFA. In a particular embodiment, the panel of VEGF biomarkers (effector signaling proteins) measured in a patient sample with a RCC solid tumor are VEGFR1, VEGFR2 and VEGFA. See, FIG. 4B and Example 3.

mTOR Pathway Biomarkers

The mTOR pathway comprises a number of molecular entities that interact in a sequential fashion to provide a signaling cascade or transduction mechanism or means. Hence, a molecule triggers or induces a change, such as, phosphorylation of a first target molecule. When a second target molecule is acted on by the, for example, phosphorylated first target molecule, the second target molecule then is changed, and as a changed molecule is enabled to trigger or to induce a change in a third target molecule, and so on. Lists of proteins that are included in the mTOR pathway can be found in commercial distributors of individual pathway components or of antibodies that bind individual pathway components, such as, Cell Signaling Technology, Inc. Danvers, Mass.; BioCarta LLC, San Diego, Calif.; and SABiosciences/Qiagen, Valencia, Calif., and include, for example, ras, p110, p85, pI3K, PTEN, Akt, PDK1, mTOR, Rictor, Raptor, IRS1, PIP2, PIP3, Proctor, mLST8, PLD1, PA, Redd1/2, FKBP12, TSC1, FKBP38, FK506, FK520, ERK, RSK1, LKB1, Sin1, AMPK, TSC1, Rheb, PRAS40, PHLPP1/2, GSK3b, PKA, 4EBP1, eiF4E, eiF4A, FOXO1, Rag A/B/C/D, SHIP1, pAKT Substrate, TSC2, p70S6K, ATG13, 4E-BP1, POC-1, S6K, Tel2, 3-RAF, PPAR, AMPK, Dv1, Hif2A, NF1, ROC1, eIF4B, S6, eEF2K, PDCD4, various GPCR's, Hif1, STK11, p53, SGK, PKC, TORK3, FKBP and so on including phosphorylated versions of these proteins, see, for example, Hernanedez-Aya et al., The Oncologist, 16:404-414, 2011; Darwish et al., J. Urology, 2013 (published online November 2012); Borders et al., Am J Health Syst Pharm, 67(24):2095-2106 (2010); WO 2007/047754. Any molecule involved in the metabolism, that is activation or inactivation, of the mTOR pathway is contemplated in the practice of the present methods.

mTOR is present in two kinase complexes: mTORC1 and mTORC2 with mTORC2 responsible for the full activation of AKT, the upstream activator of mTORC1. The mTOR signaling pathway has been shown to play a critical role in tumor growth and has become a popular target for new therapeutics.

mTOR pathway proteins, considered alone or in combination, are predictive biomarkers of tumor response to agents that target the mTOR pathway. As set forth in the Examples to follow, combining the measured expression and activation levels of different sets of mTOR pathway proteins may be used for predicting response of different tumor types. Thus, for example, a panel can be used for predicting response of kidney tumors to TORISEL and/or AFINITOR (pPRAS40, mTOR, pmTOR_Ser2448, p4EBP1_Ser65, p4EBP1_Thr37-46, pAKT substrate) (Example 4) and another panel can be used for identifying patients with breast cancer that likely will respond to a HER2 inhibitor, alone or in combination with an mTOR inhibitor (Example 6).

When a panel is used, that panel can comprise two or more, three or more, four or more, five or more, or more biomarkers, where the biomarkers comprise molecules of the mTOR pathway. However, the panel is not limited to only biomarkers of the mTOR pathway but can include other biomarkers known or found to be associated with a particular cancer or biomarkers of other interconnected signal transduction pathways.

### Kidney Cancer Biomarkers

In certain embodiments, biomarkers for assessing responsiveness of mTOR inhibitors were screened in retrospective tissue samples from patients diagnosed with advanced renal cell carcinoma. See, Examples 4 and 5.

In certain embodiments, the present methods predict responsiveness or non-responsiveness of an mTOR inhibitor on a RCC solid tumor by measuring mTOR pathway effector signaling proteins, also referred to herein as mTOR biomarkers. In one embodiment, the biomarkers demonstrating activation of the mTOR pathway in kidney cancer type solid tumors (e.g. renal cell carcinoma) may comprise any protein directly or indirectly involved with the activation of the mTOR pathway. In a particular embodiment, the present methods utilize biomarkers comprising CA IX, p-PRAS40, mTOR, p-mTOR (Ser 2448), p-4EBP1 (Ser 65), p-4EBP1 (Thr 37-46), 4EBP1, PRAS40, and p-AKT (Substrate) for demonstrating activation of the mTOR pathway in RCC solid tumors. The biomarkers may comprise two or more of any protein involved in activation of the mTOR pathway, including two or more of the above listed biomarkers. In another embodiment, the biomarkers may comprise three or more, four or more or five or more, six or more of any protein involved in the activation of the mTOR pathway, including three or more of the above listed biomarkers.

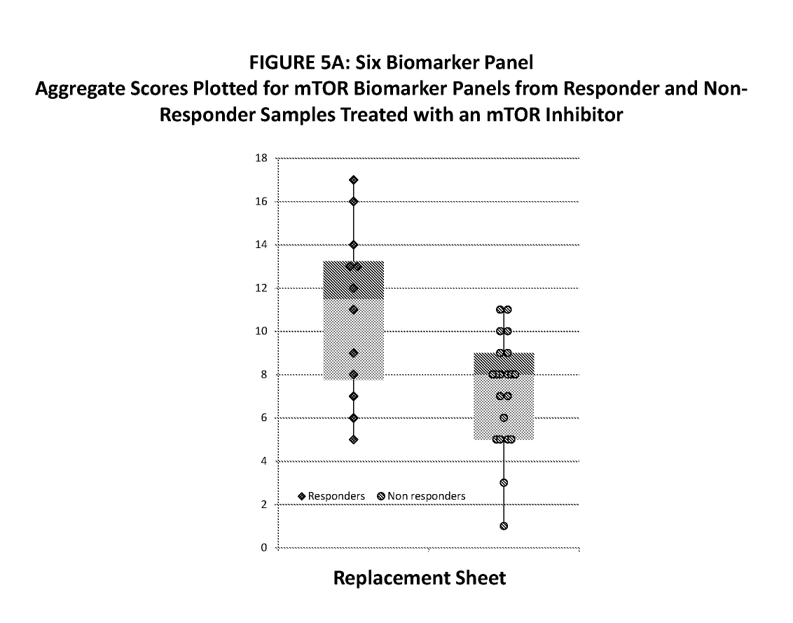

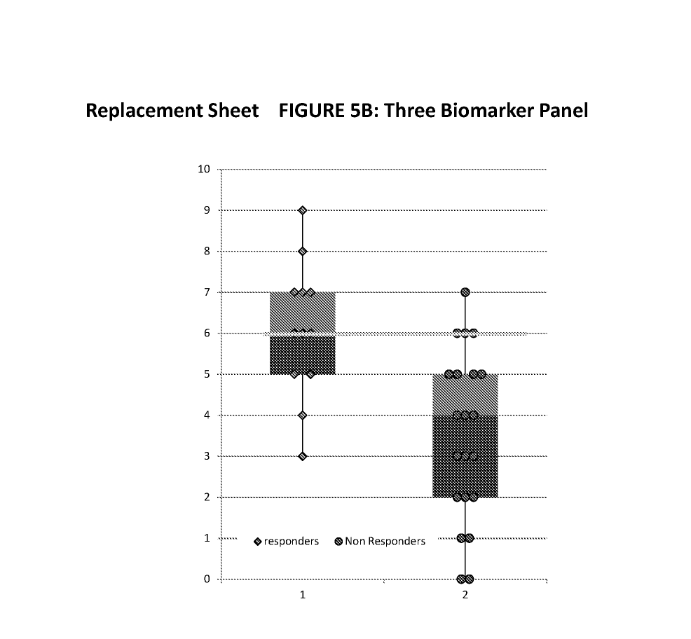

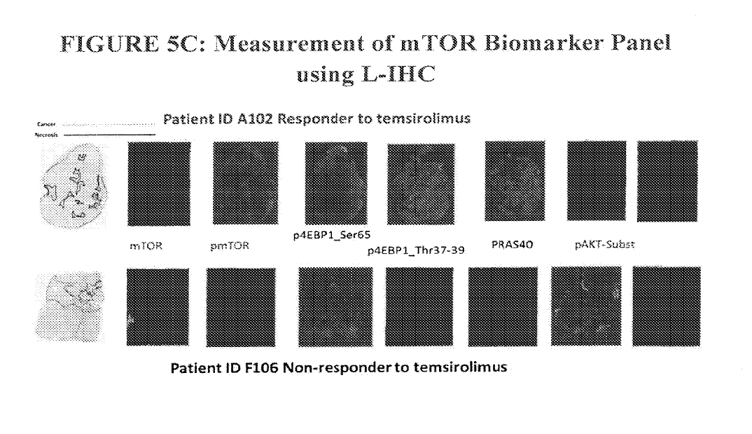

In a particular embodiment, the biomarkers may comprise mTOR, p-mTOR (Ser 2448), p-4EBP1 (Ser 65), p-4EBP1 (Thr 37/46), PRAS40, and p-AKT (Substrate). In another particular embodiment, the panel of biomarkers (effector signaling proteins) measured in a patient sample with a RCC solid tumor are mTOR, p-mTOR (Ser 2448), p-4EBP1 (Ser 65), p-4EBP1 (Thr 37/46), PRAS40, and p-AKT (Substrate). See, FIGS. 5A and 5C; Example 4.

In another particular embodiment, the biomarkers may comprise p-mTOR, p4EBP1 (Ser 65) and p4EBP1 (Thr 37/46). In another particular embodiment, the panel of biomarkers (effector signaling proteins) measured in a patient sample with a RCC solid tumor are p-mTOR, p4EBP1 (Ser 65) and p4EBP1 (Thr 37/46). See, FIG. 5B and Example 5.

These mTOR biomarkers are measured in a patient sample with an RCC solid tumor, wherein they are designated with an assigned score which may be combined to obtain an aggregate score and this aggregate score then compared to a threshold value for predicting responsiveness or non-responsiveness to an mTOR inhibitor. Depending on the analysis performed on the measurement of the biomarkers, a value above the threshold value may indicate activation of the mTOR pathway and subsequently predict responsiveness to an inhibitor of mTOR. Likewise, a value below the threshold value may indicate little or no activation of the mTOR pathway and subsequently predict non-responsiveness to an inhibitor of mTOR.

### Breast Cancer Biomarkers

In certain embodiments, biomarkers for assessing activation of the mTOR signal transduction pathway were screened in retrospective tissue samples from patients diagnosed with HER2 positive breast cancer. See, Example 6.

In certain embodiments, the present disclosure provides methods for measuring activation of the mTOR pathway in a sample obtained from a patient with a breast cancer solid tumor. In certain embodiments the solid tumor is HER2 positive and activation of the mTOR pathway in these tumors, using the present methods, is predictive that the tumor will likely be non-responsive to an inhibitor of HER2. When the mTOR pathway is shown to be activated, using the present methods, the tumor may be responsive to an mTOR inhibitor either alone or in combination with a HER2 inhibitor.

In one embodiment, the biomarkers for demonstrating mTOR activation in a breast cancer solid tumor may comprise any protein directly or indirectly involved with the activation of the mTOR pathway. In a particular embodiment, the present methods use biomarkers comprising pPTEN, p-AKT (Thr 308), p-PDK1, Her4, Muc4, HER2, vimentin, p-AKT (Ser 473), p-mTOR, p-ERK1/2, p-4EBP1, HIF 1α, mTOR, and 4EBP1 for demonstrating activation of the mTOR pathway in HER2 positive solid tumors. The biomarkers may comprise two or more of any protein involved in activation of the mTOR pathway, including two or more of the above listed biomarkers. In another embodiment, the biomarkers may comprise three or more, four or more or five or more, of any protein involved in the activation of the mTOR pathway, including three or more of the above listed biomarkers.

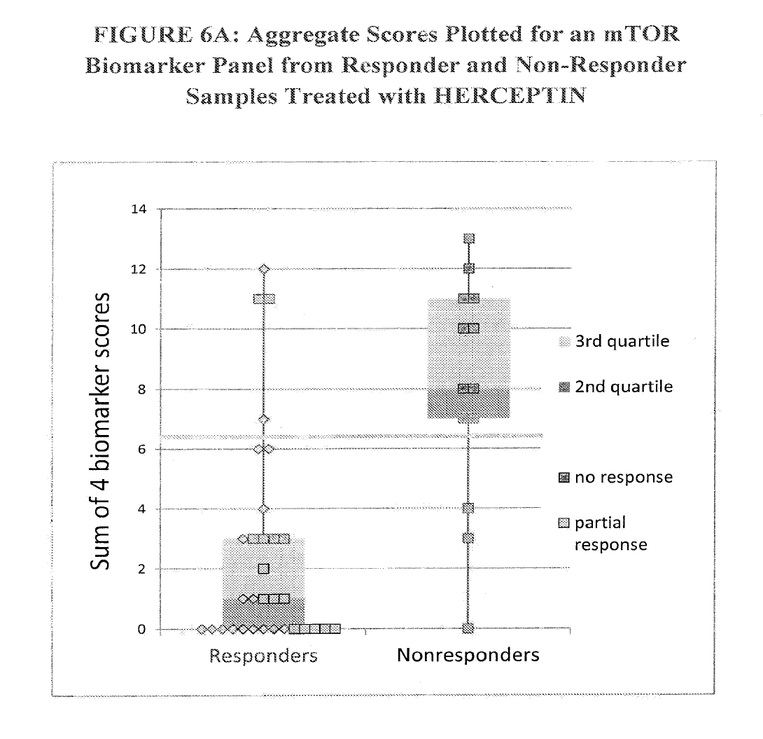

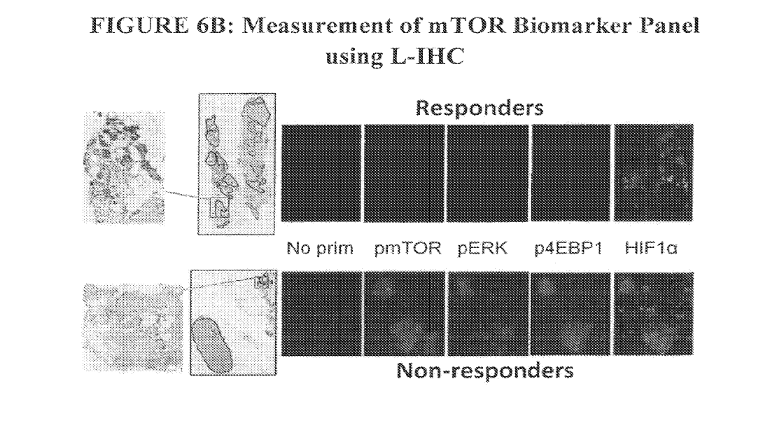

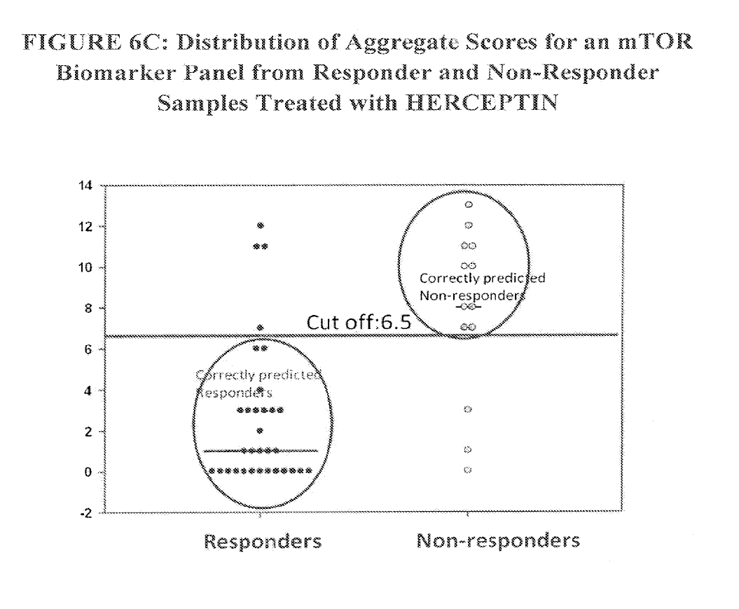

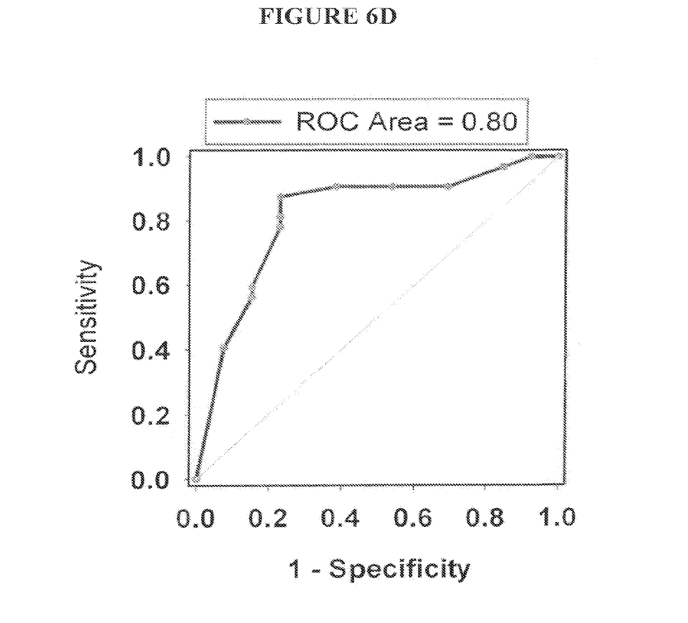

In a particular embodiment, the biomarkers may comprise p-mTOR, pERK1/2, p4EBP1 and HIF 1α. In another particular embodiment, the panel of biomarkers (effector signaling proteins) measured in a patient sample with a HER2 positive solid tumor are p-mTOR, pERK1/2, p4EBP1 and HIF 1α. See, FIG. 6 and Example 6

### 6) Development of a Predictive Algorithm Based on the Predictive Biomarkers and Retrospective Samples

The aggregate scores, described above, were analyzed using the outcome data from the retrospective samples. In this way the aggregate scores were graphed (See, FIG. 4A) against the responder and non-responder data. This resulted in the ability to identify a threshold value or predetermined cut off value for use in predicting responsiveness when patient samples are tested by identifying a predicted responder group.

In exemplary embodiments, the retrospective samples were categorized, after treatment with a respective therapeutic agent based on the response of the agent to the solid tumor, such as complete response, partial response, disease stable and non-response. In this way, pathway specific biomarkers were analyzed to determine the appropriate make up of the panel, described above, and also to generate a threshold value, wherein, for example, above the predetermined cut off predicts, or indicates prediction, that the solid tumor will respond to the therapeutic agent and a value below the predetermined cut off predicts, or indicates prediction, that the solid tumor will not respond to the therapeutic agent. It is understood that this predetermined cut off may not be an absolute value and that there may a margin of error above and below the threshold value wherein it may not be possible to accurately predict the responsiveness or non-responsiveness of the therapeutic agent on a solid tumor. Thus, in certain embodiments the present method assesses the likelihood a patient with a solid tumor will be responsive or non-responsive to a therapeutic agent that inhibits a signal transduction pathway prior to treatment with the therapeutic agent.

The threshold value is determined for each set of predictive biomarkers and for each disease tissue (e.g. RCC or breast cancer). The threshold value or predetermined cut off may be a specific number such that above and below that number an aggregate score is predictive for responsiveness or non-responsiveness of the disease tissue to the targeted therapy. For example, the predictive algorithm may provide two categories (i) likely responder and (ii) unlikely responder. In this instance, a specific number such as used in FIG. 4 delineates the predicted responders from non-responders group such that a patient sample would be tested and based on the aggregate score fall into one of these two categories for predicting response of the disease tissue to the targeted therapy. The cut off value, or grouping of aggregate scores into categories, may be done based on multiple factors. In certain embodiments, the cut off value is selected to maximize accuracy.

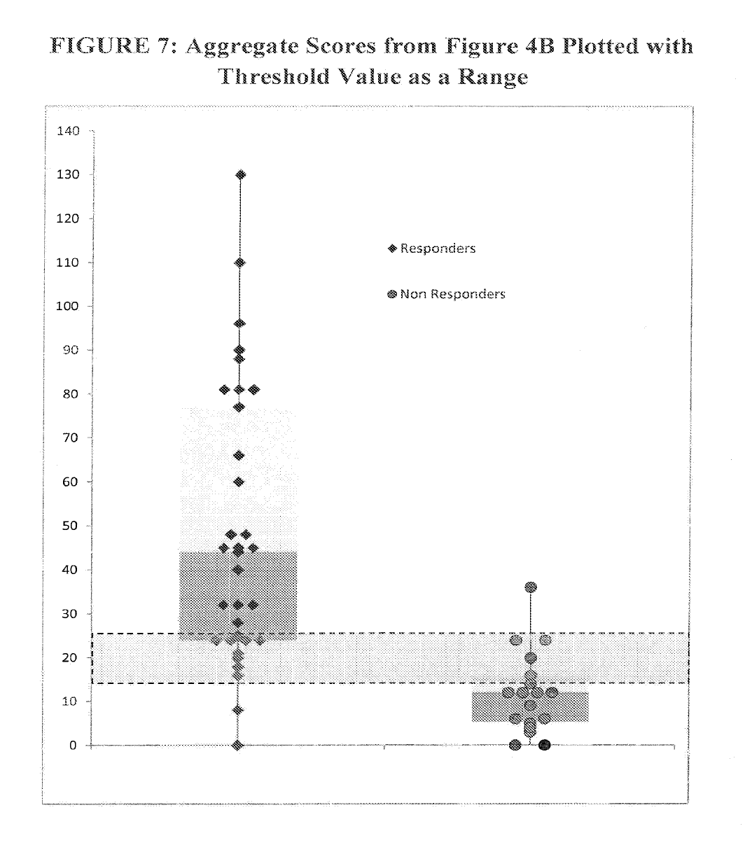

Alternatively, the predictive algorithm may provide three categories (i) likely responder, (ii) likely non-responder, and (iii) indeterminate likelihood of response. In this instance, a range of numbers would delineate the responders from the non-responders such that a patient sample tested with an aggregate score in the first two categories would be predictive for responsiveness of the targeted therapy on the disease tissue. See, FIG. 7.

In certain embodiments, the cut off between the three categories may be set such that patients with a score in the likely responder group would have better than 80% chance of responding (e.g. 8 in 10 patients would respond to the targeted therapy), better than 90% chance of responding or 100% chance of responding to the targeted therapy. In other embodiments, the cut off between the three categories may be set such that patients with a score in the likely non-responder group would have better than 80% chance of not responding (e.g. 8 in 10 patients would non-respond to the targeted therapy), better than 90% chance of not responding or better than 100% chance of not responding to the targeted therapy. In further embodiments, the cut off between the three categories may be set such that patients with a score in the indeterminate likelihood of response group would have the same likelihood of responding to the targeted therapy as before the test was performed. In other embodiments, the cut off between the three categories may be set such that patients with a score in the indeterminate likelihood of response group may have a 50% chance of responding, less than a 50% chance of responding or greater than a 50% chance of responding to the targeted therapy.

In certain other embodiments, the predictive algorithm may comprise four or more categories segregated by the percentage (accuracy) that a patient, based on their aggregate score, would be responsive to a targeted therapy. In this instance, the predictive algorithm may provide four categories (i) 100% responsive to the targeted therapy; (ii) 50% chance of being responsive to the targeted therapy; (ii) 20% chance of being responsive to the targeted therapy and (iv) 100% non-responsive to the targeted therapy. See, Example 2B and 3B. The segregation of the categories, and number of, may be accomplished in many different ways depending on the data set from the retrospective samples and the needs of the patient and/or treating physician and/or the known efficacy of the targeted therapy.

In addition to using the predictive model to predict a patients response to a targeted therapy for treatment, the model may also be used for selecting patient populations in a clinical trial to improve response rate of the drug candidate in the selected population.

7) Transformation of the Measured Biomarker Panel into a Predictive Score.

Once a threshold value or predetermined cut off is determined for a panel of biomarkers for a particular disease tissue, patient sample may be analyzed using the present methods disclosed herein to determine individual aggregate scores for each patient, also referred to herein as predictive scores.

In certain embodiments, the present methods comprise comparing the aggregate score generated from a patient sample to a data set of aggregate scores from reference samples, also referred to herein as retrospective samples, comprising a predetermined cut off for predicting responsiveness and non-responsiveness for a therapeutic agent. See Example 2-6. In this instance, the predetermined cut off is calculated from a data set of aggregate scores, wherein the aggregate scores were generated from measurement of the biomarker panel in retrospective samples and combination of the assigned scores. In certain embodiments, the aggregate scores were generated from retrospective samples (e.g., the samples were pre-classified but not disclosed to the researcher until after testing was completed). Based on this data set generated from retrospective samples a threshold value was determined based on the empirical value of the aggregate score and the responsiveness of the therapeutic agent to the solid tumor. It is understood that a threshold value, or predetermined cut off value, may be any value provided there is a good fit of data above and below that corresponds to responders and non-responders from the retrospective samples. Typically, the cut off value is selected to maximize accuracy. It is also understood that the threshold value or cut off value may be a range rather than a specific number. For example, when using the present methods with a patient sample, the threshold value may be 15-25, wherein and aggregate score below fifteen (15) predicts non-responsiveness to a targeted therapy for the signal transduction pathway being measured and an aggregate score above twenty five (25) predicts responsiveness to the targeted therapy. In this instance, an aggregate score between 15 and 25 is inconclusive and/or non-predictive. See, FIG. 7.

The patient aggregate score may also be compared to a data set comprising a four or more categories based on the percentage (%) chance a patient has for being responsive to the targeted therapy. In this instance, for example a patient having an aggregate score of 25 or higher is predicted to be responsive 100% of the time; an aggregate score of 19-24 is predicted to have a 50% chance of being responsive; an aggregate score of 14-18 is predicted to have a 20% chance of being responsive; and an aggregate score of less than 14 is predicted to be non-responsive 100% of the time. See, Example 2B and Table C.

In certain other embodiments, the present methods comprise comparing the assigned score to an index score for predicting responsiveness and non-responsiveness for a targeted therapy. See, Example 6B. In this instance, the index score is calculated from a data set of assigned scores, wherein a mean for each biomarker in each category (e.g., response, non-response) is calculated. In one embodiment, a mean value for a biomarker in the non-responder group is divided by the mean value of the same biomarker in the responder group to generate an index value for that biomarker. In certain aspects, this is repeated for each biomarker to generate a table or data set of index scores for each biomarker in the panel. See, Table 9. The present methods contemplate comparing one or more assigned scores from the panel of biomarkers measured to the data set comprising corresponding index scores to predict the responsiveness or non-responsiveness of a solid tumor to a therapeutic agent. In this instance, an assigned score for a biomarker with a value higher than the corresponding index score predicts non-responsiveness of the solid tumor to the therapeutic agent. Alternatively, an assigned score for a biomarker with a value less than the corresponding index score predicts responsiveness of the solid tumor to the therapeutic agent.

In an alternative embodiment, a mean value for a biomarker in the responder group is divided by the mean value of the same biomarker in the non-responder group to generate an index value for that biomarker. In this instance, an assigned score for a biomarker with a value higher than the corresponding index score predicts responsiveness of the solid tumor to the therapeutic agent and an assigned score for a biomarker with a value less than the corresponding index score predicts non-responsiveness of the solid tumor to the therapeutic agent.

In yet other embodiments, the present methods comprise comparing a panel of assigned scores, derived from measurement of a panel of biomarkers in a patient sample to a panel of assigned scores (optionally normalized or averaged) derived from retrospective samples. In certain aspects, the signature scores are a mean of each biomarker measured in a group (e.g. responders) related to a mean of the corresponding biomarker measured in another group (e.g. non-responders). It is understood that there are many ways, known to one of skill in the art, in which the data (e.g. measurement of biomarkers) can be analyzed (e.g. individually as a mean, as a ratio or in aggregate) and presented in a proteomic signature (panel of markers) and compared to a threshold value (collection of biomarker values from the panel) derived from retrospective data.

The predictive score, as determined using the present methods and predictive model, may then be provided to a physician and/or patient. In certain embodiments, based on the predictive score, a recommendation may be made to treat the patient with the target therapy because the patient has a predictive score corresponding to the predicted responder group. In other embodiments, based on the predictive score, a recommendation may be made that the patient not be treated with the targeted therapy because the patient has a predictive score corresponding to the predictive non-responder group of the predictive algorithm. In yet other embodiments, based on the predictive score, no recommendation may be made on treatment with the target therapy. In this instance, the patient predictive score more correspond to an indeterminate group of the predictive algorithm or a group with less than an 80% chance of responding or non-responding to the targeted therapy. Depending on the patient predictive score, it is also contemplated that a recommendation may be made that the patient be treated with the standard of care.

### D) Clinical Use of the Tests for Predicting Responsiveness or Non-Responsiveness of a Solid Tumor to a Therapeutic Agent

In use and operation, tests developed in the manner disclosed above may be used by oncologists to help them predict the responsiveness of a solid tumor to a therapeutic agent. A response criterion for patients undergoing cancer therapy has been described in the Revised RECIST guideline (Eisenhauer E A, et al. New response evaluation criteria in solid tumours: Revised RECIST guideline (version 1.1). Eur J Cancer. (2009)45:228-247) and includes complete response in patients who have complete disappearance of all lesions, partial response in patients who have at least a 30% decrease in sum of diameter of lesions, stable disease in patients who have neither sufficient shrinkage to qualify for partial response nor sufficient increase to qualify for progressive disease, and progressive disease for those patients who have at least a 20% increase in sum of diameters of lesions. In the present disclosure, the responsiveness of the therapeutic agent is predicted based on the activation of a signaling transduction pathway wherein the therapeutic agent targets or inhibits the pathway, either directly or indirectly. In one aspect, the methods predict that the solid tumor will be responsive to the therapeutic agent. In another aspect, the present methods predict that the solid tumor will be non-responsive to the therapeutic agent.

Thus, in certain embodiments the method of predicting whether a patient with a solid tumor will respond to a therapeutic agent that inhibits a signal transduction pathway; comprises 1) measuring in a patient sample two or more signaling effector proteins, wherein each measured signaling effector protein is assigned a score based on an inferred amount of protein measured; 2) combining the assigned scores to obtain an aggregate score; 3) comparing the aggregate score to a data set of aggregate scores from reference samples comprising a predetermined cut off for predicting responsiveness and non-responsiveness for a targeted therapy, wherein the reference samples are pre-classified retrospective samples from patients treated with the pathway specific drug; and, 4) providing a report comprising a treatment recommendation based on the aggregate score.

In alternative embodiments, the assigned scores are not combined, but may be further analyzed and collectively or individually compared to a threshold value for predicting responsiveness or non-responsiveness of the therapeutic agent on the solid tumor. Further analysis may comprise, but is not limited to, weighting of the assigned scores, generating a ratio of the assigned scores, etc.

In other certain embodiments, the present disclosure provides methods for predicting the likelihood a patient with a solid tumor will be responsive or non-responsive to a therapeutic agent that inhibits a signal transduction pathway. In one embodiment, the method of assessing a likelihood a patient with a solid tumor will be responsive or non-responsive to a therapeutic agent that inhibits a signal transduction pathway prior to treatment with the therapeutic agent; comprises 1) obtaining a sample of the solid tumor wherein tumor and non-tumor cells are delineated; 2) measuring in the sample two or more signaling effector proteins from a signal transduction pathway, wherein each measured signaling effector protein is assigned a score based on an inferred amount of the protein measured; 3) combining the assigned scores from the tumor cells to obtain an aggregate score; 4) comparing the aggregate score to a predetermined cut off for predicting responsiveness and non-responsiveness for the therapeutic agent, whereby the likelihood the patient will be responsive or non-responsive to the therapeutic agent that inhibits the signal transduction pathway prior is assessed.

A tumor is considered to be responsive if it displays sensitivity to an inhibitor of a signal transduction pathway (e.g. VEGF receptor or mTOR inhibitor) or if it possesses characteristics such that it will display sensitivity to an inhibitor of a signal transduction pathway when exposed to such an inhibitor. A tumor is considered to be non-responsive or resistant if it is currently displaying resistance (lack of sensitivity) to an inhibitor of a signal transduction pathway (e.g. HER2 inhibitor) or if it possesses characteristics such that it will display resistance to an inhibitor of a signal transduction pathway when exposed to such an inhibitor. One of ordinary skill in the art will recognize that a method for evaluating the likelihood that a tumor is sensitive to an inhibitor of a signal transduction pathway also evaluates the likelihood that the tumor is resistant to an inhibitor of a signal transduction pathway. Similarly, one of ordinary skill in the art will recognize that a method for evaluating the likelihood that a subject will exhibit a favorable response to an inhibitor of a signal transduction pathway (e.g. a VEGF receptor inhibitor) also evaluates the likelihood that the subject will not exhibit a favorable response to such an inhibitor. For purposes of convenience, the present application refers primarily to methods for evaluating the likelihood that a tumor is sensitive to an inhibitor of a signal transduction pathway and/or that a subject will exhibit a favorable response to an inhibitor of a signal transduction pathway. Such methods are considered equivalent to methods for evaluating the likelihood that a tumor is resistant to an inhibitor of a signal transduction pathway and/or that a subject will not exhibit a favorable response to an inhibitor of a signal transduction pathway since the information obtained by practicing the methods can be expressed in any of these various terminologies.

One or more steps of the method described herein can be performed manually or can be completely or partially automated (for example, one or more steps of the method can be performed by a computer program or algorithm. If the method were to be performed via computer program or algorithm, then the performance of the method would further necessitate the use of the appropriate hardware, such as input, memory, processing, display and output devices, etc). Methods for automating one or more steps of the method would be well within the skill of those in the art.

In yet further embodiments, the present invention contemplates specific use computer, which may be a general purpose computer, configured to perform the steps of the method described herein. The method, or portions of the method, may be further embodied in a computer readable medium capable of being executed in a computer environment. Such computer readable medium may be a specific storage device, such as a disk, or a location on a server, physical or virtual, the storage device may be accessed by a computer for performing the required steps of the method.

### 1) Measuring Biomarkers in a Patient Sample

The first steps in the present method comprise obtaining a sample comprising solid tumor cells and measuring a panel (e.g., two or more) of markers in the sample. The biomarkers may be measured using any of the methods disclosed above and/or well known in the art for measuring gene expression or protein expression.

In exemplary embodiments immunohistochemistry (IHC) is used to measure the biomarkers in a patient sample. In a particular embodiment, the IHC is L-IHC disclosed herein.

Patient samples may be acquired and the biomarkers measured at the same location. In an alternative embodiment, the patient sample is acquired and sent to a different location for the measurement of the biomarkers.

Reagents (typically antibodies) for detecting the biomarkers are usually commercially available, as are secondary antibodies to serve a reporter function, if the primary antibodies are not labeled. For example, kits for detecting carbonic anhydrase IX are commercially available (R & D Systems, Minneapolis, Minn.). Antibodies to the various mTOR pathway molecules and/or VEGF are available commercially, as noted hereinabove, and in the working examples below.

Alternatively, antibodies to a biomarker can be made practicing methods known in the art. The antibody can be monoclonal. The antibody can comprise only a portion of an intact immunoglobulin, such as, only the antigen-binding portion of the molecule, such as, the Fab portion of the molecule. The antibody can be recombinant, in part or in full. The antibody can be labeled practicing methods known in the art, using reporters known in the art.

### 2) Signal Transduction Activation Pathway Biomarkers

However, before measurement can be performed a panel (e.g. two or more) of biomarkers needs to be selected for a particular signal transduction pathway associated with a solid tumor being screened. Many markers are known from signal transduction pathways associated cancers and a known panel can be selected, or as was done by the present Applicants, a panel can be selected based on measurement of individual markers in retrospective clinical samples wherein a panel is generated based on empirical data for a solid tumor, signal transduction pathway and a therapeutic agent that targets or inhibits that pathway.

The present methods contemplate any panel of biomarkers, when measured and taken individually, collectively or in aggregate, can be used in the present methods to predict responsiveness of a solid tumor to a therapeutic agents that inhibits a signal transduction pathway.

The signal transduction pathway includes any pathway involved in growth (e.g. proliferation or angiogenesis) or maintenance (e.g. enzyme metabolism) of a solid tumor. It is understood that the signal transduction pathways are broad and often interconnected and as such the nomenclature for referring to such a pathway may be by the receptor (e.g. EGFR), the drug target (mTOR), or the ligand or factor (e.g. IL-8). The drug target may be the receptor or the ligand, or any other protein in the cascade that if inhibited or blocked would lead to disruption of the signal transduction pathway. There is no intended limitation on the signal transduction pathway in the present methods and such pathways include, but are not limited to, PI3K/AKT/mTOR, HER2, HER3, VEGF, HIF, Ang-2, EGFR, PDGF, PDGFR, EGF, TGF-beta, FGF, FGFR, NGF, TGF-alpha, IGF-I, IGF-II, and IGFR. Signal transduction pathways may also be generally referred to as cytokine pathways, receptor tyrosine kinase (RKT) pathways, MAPK pathways, etc.

### a) VEGF Pathway Biomarkers

In certain embodiments, the biomarkers are VEGF pathway biomarkers. In particular embodiments, the VEGF biomarkers comprise a panel of VEGF biomarkers disclosed above. These VEGF biomarkers are measured in a patient sample with a solid tumor, wherein they are designated with an assigned score which may be combined to obtain an aggregate score and this aggregate score then compared to a threshold value for predicting responsiveness or non-responsiveness to a VEGF inhibitor. Depending on the analysis performed on the measurement of the biomarkers, a value above the threshold value, which may be a specific number or a range of numbers (e.g. 15 to 20 as the threshold value) may indicate activation of the VEGF pathway and subsequently predict responsiveness to an inhibitor of VEGF. Likewise, a value below the threshold value may indicate little or no activation of the VEGF pathway and subsequently predict non-responsiveness to an inhibitor of VEGF.

Thus, in certain embodiments, the present disclosure provides methods for predicting whether a patient with a solid tumor will respond to a therapeutic agent that inhibits a VEGF pathway, comprising: 1) measuring in a patient sample two or more VEGF signaling effector proteins, wherein each measured VEGF signaling effector protein is assigned a score based on an inferred amount of protein measured; 2) combining the assigned scores to obtain an aggregate score; 3) comparing the aggregate score to a data set of aggregate scores from reference samples comprising a predetermined cut off for predicting responsiveness and non-responsiveness for a targeted therapy, wherein the reference samples are pre-classified retrospective samples from patients treated with the therapeutic agent that inhibits the VEGF pathway; and, providing a report comprising a treatment recommendation based on the aggregate score.

In other certain embodiments, the present disclosure provides methods for assessing a likelihood a patient with a solid tumor will be responsive or non-responsive to a therapeutic agent that inhibits a VEGF pathway prior to treatment with the therapeutic agent, comprising: 1) obtaining a sample of the solid tumor wherein tumor and non-tumor cells are delineated; 2) measuring in the sample two or more VEGF signaling effector proteins, wherein each measured VEGF signaling effector protein is assigned a score based on an inferred amount of the protein measured: 3) combining the assigned scores from the tumor cells to obtain an aggregate score; 4) comparing the aggregate score to a predetermined cut off for predicting responsiveness and non-responsiveness for the therapeutic agent, whereby the likelihood the patient will be responsive or non-responsive to the therapeutic agent that inhibits the VEGF pathway is assessed.

### i) Renal Cell Carcinoma

One specific example of clinical use of the present invention is with advanced renal cell carcinoma (RCC), a particularly aggressive cancer. Due to the lack of early symptoms or detectable metabolic abnormalities by diagnostic assays in early stages of the disease, despite improvements of imaging techniques, only 60% of RCC detected are localized. Moreover, among those patients, one third to one half will develop distant metastases within one year following surgery.

In 2009, it was estimated that 40,000 new cases of RCC would be identified in Europe and 23,000 in China with an estimated death rate of about 40%. It is estimated that 57,760 Americans were diagnosed with kidney cancer in 2009 and 12,980 individuals are expected to succumb to the disease. In the US, RCC represents 3% of cancer incidence and mortality, yet RCC is the 6^{th} leading cause of cancer death. Diagnosis of kidney cancer is on the rise primarily due to incidental findings owing to increased use of abdominal imaging.

Particularly resistant to chemotherapy and radiation, until recently, the only available treatment of RCC after surgery was immunotherapy, interferon α (INFα) or interleukin-2 (IL2), with, however, limited success. Recently, several molecularly targeted therapies have been approved for both first and second-line treatment of RCC. Those targeted therapies include the multikinase inhibitors, sunitinib (SUTENT®), sorafenib (NEXAVAR®) and pazopanib (VOTRIENT®), the mTOR inhibitors, temsirolimus (TORISEL®) and evirolimus (AFINITOR®), and the anti-VEGF-A monoclonal antibody, bevacizumab (AVASTIN®). However, not all patients are responsive to any one of those new drugs. The mTOR inhibitors, in particular, benefit a smaller subset of patients to whom they are administered, in some cases as low as 10%.

Thus, a sometimes large proportion of candidate kidney cancer patients, may not respond or may acquire resistance to a particular form of therapy at the onset of treatment or after treatment begins. The lack of responsiveness not only delays effective treatment, but incurs costs and impacts patient health and morale.

Heretofore, there are no diagnostic tests being utilized to indicate which of those therapies is best suited for a particular patient. The actions of molecularly-targeted drugs are dependent on, for example, a molecular defect within the signaling pathway that is targeted by the drug; in particular, expression levels of the drug targets in tumor tissue; on the activities of molecules involved with any molecular pathways associated with the target; and so on. As such, measurement of the expression levels or the relative levels of many different molecules in a particular signaling pathway may be relevant to the prediction of drug efficacy in a certain patient. Teh et al., U.S. Publ. No. 2009/0285832 disclose detecting IL-8 or MMP12 expression levels in a renal tumor. Elevated levels of either were alleged to correlate with non-responsiveness to sunitinib treatment.

The present methods, while demonstrating activation of the VEGF pathway in a RCC solid tumor, predict that the patients with these tumors may likely benefit from therapy with a VEGF inhibitor, either alone or as an adjuvant therapy.

In certain embodiments, the present disclosure provides methods for measuring activation of the VEGF pathway in a sample obtained from a patient with a solid RCC tumor. In this instance, the activation of the VEGF pathway is predictive of the responsiveness or non-responsiveness of a VEGF inhibitor on a RCC solid tumor.

Current guidelines for advanced RCC treatment include a first line treatment and second line treatment followed by a third or subsequent treatment if needed. While there are factors a treating physician may use to decide starting a patient on a first line or second line treatment, or when to switch from first to second, there are no marketed biomarker tests for testing patient samples that would predict responsiveness of one treatment over another treatment. As an example, in the case of SUTENT, assuming a 30% efficacy, if all advanced RCC patients were first treated with the drug, out of 100 patients only 30 would respond and 70 would be non-responsive to the drug. The present methods and predictive model, when utilized with patient samples, can increase the efficacy of SUTENT by pre-selecting those 30% responders (e.g. the patient population) that will respond to the drug. In this way, a higher percentage of responders would be selected and recommended for SUTENT treatment, increasing the efficacy in the treatment group. The patient group with predictive scores corresponding to the predictive non-responder group may still be treated with SUTENT or the treating physician may elected, based on the predictive score, to treat the patient with another drug that may be more efficacious for that particular patient.

In certain embodiments, the present tests increase the efficacy of SUTENT in a treated patient population by 20%, by 30%, by 40%, by 50%, by 60%, by 70%, by 80%, by 90% or by greater than 100%. The present tests and predictive algorithm identify those patients diagnosed with advanced RCC that have a better than 30% chance of responding to the targeted therapy (e.g. SUTENT). In other embodiments, when SUTENT is administered based on the present test, the efficacy of SUTENT in the predicted responder group may have an efficacy of greater than 40%, greater than 50%, greater than 60%, greater than 70%, greater than 80% or greater than 90% in that patient group. In particular embodiments, the efficacy of SUTENT may be increased to greater than 60% (See, Example 2B) or greater than 80% (See, Example 3B) in the treated patient population.

Efficacy may also be stated as response rate, wherein when SUTENT is administered based on the present test, the response rate in the selected patient population (predicted response group) to SUTENT is improved. In certain embodiments, the response rate in the predicted responder group to SUTENT is greater than 40%, greater than 50%, greater than 60%, greater than 70%, greater than 80% or greater than 90%. In other embodiments, the present tests improve (above 30%) the response rate of SUTENT in a treated patient population by 20%, by 30%, by 40%, by 50%, by 60%, by 70%, by 80%, by 90% or by greater than 100% (2× the number of responders). In other embodiments, the response rate of SUTENT is 2×, 3×, or greater than 3× more than 30% seen in the advanced RCC patient population before segmentation by the present tests.

In one embodiment, is provided a method for predicting whether a patient diagnosed with a solid renal cell carcinoma (RCC) tumor will respond to a therapeutic agent that inhibits a VEGF pathway, comprising: 1) measuring in a patient sample two or more VEGF signaling effector proteins, wherein each measured VEGF signaling effector protein is assigned a score based on an inferred amount of protein measured; 2) combining the assigned scores to obtain an aggregate score; 3) comparing the aggregate score to a data set of aggregate scores from reference samples comprising a predetermined cut off for predicting responsiveness and non-responsiveness for the therapeutic agent; and, 4) providing a report comprising a treatment recommendation for the patient diagnosed with the solid renal cell carcinoma (RCC) tumor based on the aggregate score.

In another embodiment is provided a method for assessing a likelihood a patient diagnosed with a solid renal cell carcinoma (RCC) tumor will be responsive to a therapeutic agent that inhibits a VEGF pathway prior to treatment with the therapeutic agent, comprising: 1) obtaining a sample of the solid tumor wherein tumor and non-tumor cells are delineated; 2) measuring in the sample two or more VEGF signaling effector proteins, wherein each measured VEGF signaling effector protein is assigned a score based on an inferred amount of the protein measured; 3) combining the assigned scores from the tumor cells to obtain an aggregate score; 4) comparing the aggregate score to a predetermined cut off for predicting responsiveness and non-responsiveness for the therapeutic agent, whereby the likelihood the patient diagnosed with solid RCC tumor will be responsive or non-responsive to the therapeutic agent that inhibits the VEGF pathway is assessed.

In exemplary embodiments, the VEGF biomarkers may comprise p-PRAS40, VEGFA, VEGFR1, VEGFR2 and PDGFRβ. In a particular embodiment, the panel of VEGF biomarkers (effector signaling proteins) measured in a patient sample with a RCC solid tumor are p-PRAS40, VEGFA, VEGFR1, VEGFR2 and PDGFR. See, FIGS. 4A and 4C; Example 2.

In another exemplary embodiment, the VEGF biomarkers may comprise VEGFR1, VEGFR2 and VEGFA. In a particular embodiment, the panel of VEGF biomarkers (effector signaling proteins) measured in a patient sample with a RCC solid tumor are VEGFR1, VEGFR2 and VEGFA. See, FIG. 4B and Example 3.

In particular embodiments, the present methods are used to predict the responsiveness of sunitinib (SUTENT) on a solid tumor of advanced renal cell carcinoma by demonstrating up- or down regulation of proteins in the VEGF pathway.

Use of biomarker or biomarker panels as described herein can help identify those patients with a VEGF-related cancer who are most likely to benefit from VEGF inhibitors, such as, sunitinib or bevacizumab, to permit the clinician to administer those drugs specifically to the patient most likely to respond.

Any number of biomarkers as disclosed herein can be employed in an assay as a design choice, seeking, for example, to maximize confidence in the results of the assay or in the power of an assay to serve as a screening assay to identify as many candidates as possible. Hence, an assay of interest may ask for presence of at least one of two or more biomarkers, three or more biomarkers, four or more biomarkers, five or more biomarkers or more as a design choice. Also, the assay can be configured to have design choice levels of sensitivity and/or specificity.

Thus, a sample from the kidney cancer patient is obtained and is exposed to the appropriate reagent or reagents for detecting one or more of the markers of interest. Methods known in the art that can be used to detect binding of the reagent to the marker or to detect an observable manifestation, such as, light, radioactivity, color and so on as known in the art, arising from binding of the reagent to the target, such as, an increase or loss of a function or result, presence, absence or varying levels of the marker of interest, and so on. When the determination is completed and the presence or absence, or inferred level of or presence of a marker is obtained, that result, whether qualitative or quantitative is compared to accumulated results obtained during developmental experiments and in patient sampling that provide a mean, average, median and so on statistic that correlates to a statistically significant likelihood that the patient will respond to a drug and/or to a statistic that correlates to a statistically significant likelihood that the patient will not respond to a drug.

As discussed above, the present tests and predictive model, when applied to a patient population and a targeted therapy administered based on the results of the predictive test increase the efficacy and/or response rate of the targeted therapy in the treated group. In certain embodiments, the present tests increase the efficacy of a targeted therapy in a treated patient population by 10%, by 20%, by 30%, by 40%, by 50%, by 60%, by 70%, by 80%, by 90% or by greater than 100%. The present tests and predictive algorithm identify those patients diagnosed with advanced RCC that have a better chance of responding than the known efficacy for the targeted therapy (e.g. VEGFR inhibitor). In other embodiments, when a VEGFR inhibitor is administered based on the present test, the efficacy of the VEGFR inhibitor in the predicted responder group may have an efficacy and/or response rate of greater than 40%, greater than 50%, greater than 60%, greater than 70%, greater than 80% or greater than 90% in that patient group.

In further embodiments, the present tests and predictive model may also be utilized for patient selection in a clinical trial setting. Demonstrating overall survival (e.g. response rate or efficacy) better than standard of care (e.g. SUTENT) may depend on part in selection of those patients that will most likely respond to a VEGF inhibitor. One such example is Linifanib (ABT-869), which showed good results in Phase I and II (Drugs R D 2010; 10(2)), however the Phase III trials have been terminated with no results reported indicating end-points were not met. Thus, in certain embodiments, the present tests and predictive models may be used to select patients during clinical trial for a VEGFR inhibitor when showing superiority to standard of car and/or other VEGFR inhibitors.

b) mTOR Pathway Biomarkers

In certain embodiments, the present disclosure provides methods for predicting whether a patient with a solid tumor will respond to a therapeutic agent that inhibits an mTOR pathway, comprising: 1) measuring in a patient sample two or more mTOR signaling effector proteins, wherein each measured mTOR signaling effector protein is assigned a score based on an inferred amount of protein measured; 2) combining the assigned scores to obtain an aggregate score; 3) comparing the aggregate score to a data set of aggregate scores from reference samples comprising a predetermined cut off for predicting responsiveness and non-responsiveness for a targeted therapy, wherein the reference samples are pre-classified retrospective samples from patients treated with the therapeutic agent that inhibits the mTOR pathway; and, providing a report comprising a treatment recommendation based on the aggregate score.

In other certain embodiments, the present disclosure provides methods for assessing a likelihood a patient with a solid tumor will be responsive or non-responsive to a therapeutic agent that inhibits a mTOR pathway prior to treatment with the therapeutic agent, comprising: 1) obtaining a sample of the solid tumor wherein tumor and non-tumor cells are delineated; 2) measuring in the sample two or more mTOR signaling effector proteins, wherein each measured mTOR signaling effector protein is assigned a score based on an inferred amount of the protein measured; 3) combining the assigned scores from the tumor cells to obtain an aggregate score; 4) comparing the aggregate score to a predetermined cut off for predicting responsiveness and non-responsiveness for the therapeutic agent, whereby the likelihood the patient will be responsive or non-responsive to the therapeutic agent that inhibits the mTOR pathway is assessed.

Panels of biomarkers with clinical utility in predicting therapeutic response can be identified using the aforementioned methods to any of a variety of solid tumors, especially those for which an mTOR inhibitor is being used or studied to treat. These include, without limitation, kidney, breast, soft tissue, brain, pancreas, and gastric cancers. In some cases activation of the mTOR pathway biomarkers in a tumor should be tested along with one or more other targets or pathways (e.g. HER2) to determine whether a combination of targeted therapies (e.g. a HER2 inhibitor together with an mTOR inhibitor) is most optimal for a particular patient (see Example 6).

### i) Renal Cell Carcinoma

The present methods, while demonstrating activation of the mTOR pathway in a RCC solid tumor, predict that the patients with these tumors may likely benefit from therapy with an mTOR inhibitor, either alone or as an adjuvant therapy.

In one embodiment, the present disclosure provides methods for measuring activation of the mTOR pathway in a sample obtained from a patient with a RCC solid tumor. In this instance, the activation of the mTOR pathway is predictive of the responsiveness of an mTOR inhibitor on a RCC solid tumor. Alternatively, if activation of the mTOR pathway is not shown this is predictive of the non-responsiveness of an mTOR inhibitor on a RCC solid tumor.

In one embodiment, the present disclosure provides methods for predicting whether a patient diagnosed with a solid renal cell carcinoma (RCC) tumor will respond to a therapeutic agent that inhibits a mTOR pathway, comprising: 1) measuring in a patient sample two or more mTOR signaling effector proteins, wherein each measured mTOR signaling effector protein is assigned a score based on an inferred amount of protein measured; 2) combining the assigned scores to obtain an aggregate score; 3) comparing the aggregate score to a data set of aggregate scores from reference samples comprising a predetermined cut off for predicting responsiveness and non-responsiveness for the therapeutic agent; and, 4) providing a report comprising a treatment recommendation for the patient diagnosed with the solid renal cell carcinoma (RCC) tumor based on the aggregate score.

In another embodiment, the present disclosure provides methods for assessing a likelihood a patient diagnosed with a solid renal cell carcinoma (RCC) tumor will be responsive to a therapeutic agent that inhibits a mTOR pathway prior to treatment with the therapeutic agent, comprising: 1) obtaining a sample of the solid tumor wherein tumor and non-tumor cells are delineated; 2) measuring in the sample two or more mTOR signaling effector proteins, wherein each measured mTOR signaling effector protein is assigned a score based on an inferred amount of the protein measured; 3) combining the assigned scores from the tumor cells to obtain an aggregate score; 4) comparing the aggregate score to a predetermined cut off for predicting responsiveness and non-responsiveness for the therapeutic agent, whereby the likelihood the patient diagnosed with solid RCC tumor will be responsive or non-responsive to the therapeutic agent that inhibits the mTOR pathway is assessed.

In a particular embodiment, the biomarkers may comprise mTOR, p-mTOR (Ser 2448), p-4EBP1 (Ser 65), p-4EBP1 (Thr 37/46), PRAS40, and p-AKT (Substrate). In another particular embodiment, the panel of biomarkers (effector signaling proteins) measured in a patient sample with a RCC solid tumor are mTOR, p-mTOR (Ser 2448), p-4EBP1 (Ser 65), p-4EBP1 (Thr 37/46), PRAS40, and p-AKT (Substrate). See, FIGS. 5A and 5C; Example 4.

In another particular embodiment, the biomarkers may comprise p-mTOR, p4EBP1 (Ser 65) and p4EBP1 (Thr 37/46). In another particular embodiment, the panel of biomarkers (effector signaling proteins) measured in a patient sample with a RCC solid tumor are p-mTOR, p4EBP1 (Ser 65) and p4EBP1 (Thr 37/46). See, FIG. 5B and Example 5.

In particular embodiments, the present methods are used to predict responsiveness of temsirolimus (TORISEL®) on a solid tumor of advance renal cell carcinoma by demonstrating activation of the mTOR pathway.

In another particular embodiment, the present methods are used to predict responsiveness of Everolimus (AFINITOR) on a solid tumor of advance renal cell carcinoma by demonstrating activation of the mTOR pathway.

Each of TORISEL and AFINITOR have a low response rate in patients diagnosed with advanced RCC, in the case of TORISEL the response rate is usually less than 10% in that patient population. mTOR inhibitors may be effective second line treatment for those patients who have failed a VEGF inhibitor, or in certain circumstances an mTOR inhibitor would be a better first line treatment than a VEGF inhibitor to treat advanced RCC. The present tests and predictive algorithm is useful for selecting those patients that would benefit from treatment with an mTOR inhibitor. In this instance, identifying those patients that would be responsive to an mTOR inhibitor would be beneficial to the patient.

As disclosed above for SUTENT, when an mTOR inhibitor (TORISEL) is administered based on the present test, the efficacy and/or response rate in the predicted responder group may be improved. In certain embodiments, the efficacy and/or response rate to the mTOR inhibitor may be improved by 10%, by 20%, by 30%, by 40%, by 50%, by 60%, by 70%, by 80%, by 90% or by greater than 100% (2× the number of responders compared to the unselected patient population). In other embodiments, the efficacy and/or response rate to the mTOR inhibitor in the predicted responder group is at least 20%, at least 30%, at least 40%, at least 50%, at least 60%, at least 70%, at least 80%, and at least 90%. In yet other embodiments, the response rate of an mTOR inhibitor may be 2×, 3×, 4×, 5×, 6×, 7×, or greater than 8× more than the known response rate for the mTOR inhibitor in the advanced RCC patient population before segmentation by the present tests.

### ii) Breast Cancer

Around 230,000 new cases of breast cancers are diagnosed and about 40,000 patients die of breast cancer annually in the USA. Routinely, HER2 positive patients represent approximately 20-25% of all breast cancer. Also, about 60% of breast cancers are estrogen sensitive. It is known that such hormone receptors are inducers of the mTOR pathway.

A percentage of breast cancers over-express HER2 and those cancers correlate with poor prognosis. A current treatment option is use of molecules, such as, monoclonal antibodies that bind HER2, such as, trastuzumab. Trastuzumab can be administered alone or in combination with other chemotherapeutic agents (Gori et al., Ann Oncol 10:648-654, 2009). However, only about 40% of HER2 positive patients respond to the costly Herceptin or Herceptin adjuvant treatment of $60,000-130,000 for quality adjusted life year (Jeyakumar & Younis, Clinical Medicine Insights: Oncology 2012:6 179-187).

A significant proportion (30% or more) of HER2 over-expressing breast cancers, however, are refractory, do not respond or acquire resistance to HER2 targeting (trastuzumab) therapy at the onset of treatment or within a year of treatment. The lack of responsiveness not only delays effective treatment, but incurs costs and impacts patient health and morale. It is believed that hyperactivity of the PI3K/AKT pathway confers trastuzumab (HERCEPTIN) resistance, and mTOR is a major downstream effector of PI3K/AKT. Preclinical studies have shown that mTOR inhibition sensitizes HER2 over-expressing tumors to respond to trastuzumab, see, e.g. Clin Cancer Res. (2009)15(23):7266-7276. Moreover, human trials have demonstrated that trastuzumab in combination with the mTOR inhibitor everolimus results in clinical benefit and disease response in patients with trastuzumab resistant HER2 over-expressing metastatic breast cancer. See e.g. J Clin Oncol 29:3126-3132. Thus, a biomarker panel identified from HER2 positive breast tumors according to the present methods may be employed to identify patients who are more likely to benefit from a combination of a HER2 inhibitor (e.g. HERCEPTIN or TYKERB) together with an mTOR inhibitor (e.g. AFINITOR or TORISEL) rather than a HER-2 inhibitor alone. The 4 member biomarker panel listed in Table 3 is one such example of such a panel.

The present methods, while demonstrating dual activation of the mTOR pathway in a HER2 positive tumor, predict that the patients with these tumors may likely benefit from adjuvant treatment comprising an mTOR inhibitor.

In certain other embodiments, the present tests and predictive algorithm may also be used on HER2 negative breast cancer to increase response rate in those patients treated with an mTOR inhibitor (e.g. TORISEL).

In one embodiment is provided a method for predicting whether a patient diagnosed with a HER2 positive solid tumor will be non-responsive to a targeted therapy with a HER2 pathway specific drug, comprising: 1) measuring in a patient sample two or more mTOR signaling effector proteins, wherein each measured mTOR signaling effector protein is assigned a score based on an inferred amount of protein measured; 2) combining the assigned scores to obtain an aggregate score; 3) comparing the aggregate score to a data set of aggregate scores from reference samples comprising a predetermined cut off for predicting responsiveness and non-responsiveness for a targeted therapy; and, 4) providing a report comprising a treatment recommendation for the patient diagnosed with a HER2 positive solid tumor based on the aggregate score.

In another embodiment is provided a method for assessing a likelihood a patient diagnosed with a HER2 positive solid tumor will be non-responsive to a therapeutic agent that inhibits a HER2 pathway prior to treatment with the therapeutic agent, comprising: 1) obtaining a sample of the solid tumor wherein tumor and non-tumor cells are delineated; 2) measuring in the sample two or more mTOR signaling effector proteins, wherein each measured mTOR signaling effector protein is assigned a score based on an inferred amount of the protein measured; 3) combining the assigned scores from the tumor cells to obtain an aggregate score; 4) comparing the aggregate score to a predetermined cut off for predicting responsiveness and non-responsiveness for the therapeutic agent, whereby the likelihood the patient diagnosed with the HER2 solid tumor will be non-responsive to the therapeutic agent that inhibits the HER2 pathway is assessed.

In a particular embodiment, the biomarkers may comprise p-mTOR, pERK1/2, p4EBP1 and HIF 1α. In another particular embodiment, the panel of biomarkers (effector signaling proteins) measured in a patient sample with a HER2 positive solid tumor are p-mTOR, pERK1/2, p4EBP1 and HIF 1α. See, FIG. 6 and Example 6

These mTOR biomarkers are measured, wherein they are designated with an assigned score which may be combined to obtain an aggregate score and this aggregate score then compared to a threshold value for predicting responsiveness or non-responsiveness to a HER2 inhibitor. Depending on the analysis performed on the measurement of the biomarkers, a value above the threshold value may indicate activation of the mTOR pathway and subsequently predict non-responsiveness to an inhibitor of HER2. Likewise, a value below the threshold value may indicate little or no activation of the mTOR pathway and subsequently predict complete or partial responsiveness to an inhibitor of HER2. Alternatively, if the HER2 positive solid tumor is predicted to be non-responsive to a HER2 inhibitor the tumor may be predicted to be responsive to an mTOR inhibitor, either alone or in combination with a HER2 inhibitor.

In particular embodiments, the present methods are used to predict non-responsiveness of trastuzumab (HERCEPTIN) on a HER2 positive solid tumor by demonstrating activation of the mTOR pathway.

In another particular embodiment, the present methods are used to predict responsiveness of an mTOR inhibitor on a HER2 positive solid tumor by demonstrating activation of the mTOR pathway.

### 3) Scoring and Biostatistics

Following patient sample acquisition and measuring of the relevant biomarkers, biostatistics is applied to the absence, presence or inferred amount of presence of the biomarkers to calculate a predictive score. As described above, in particular embodiments the measured biomarkers are individually assigned a score following measurement wherein the assigned score is based on a graded scale and the value assigned (e.g. zero to four) is designated to each biomarker measurement based on an inferred and/or relative amount of biomarker measured in the sample. See FIG. 1 and Example 1 for exemplary assigned scoring methods.

In certain embodiments, the graded scale comprises zero to four, zero to 10; zero to 12; zero to 20; or some combination thereof. In an alternative embodiment the scale starts with 1 and not zero, either way, the smallest integer designates the absence of a biomarker (as evidenced by a lack of a signal in the methods used to measure the biomarker) and the largest designates a high for the measured biomarker.

In certain embodiments, these assigned scores are combined to obtain an aggregate score. In this instance the aggregate score is compared against a pre-determined cut off for predicting responsiveness or non-responsiveness of a therapeutic agent on a solid tumor. In certain other embodiments, the assigned scores are not combined, but individually or collectively as a proteomic signature, either before or after further application of biostatistics, used to calculate a predictive score. In this instance, a pre-determined cut-off, either individually for each marker or collectively, is applied to calculate a predictive score for each patient sample with a solid tumor cells.

As described above, the biomarkers are measured by methods well known in the art, including acquisition of an image such as with IHC. In exemplary embodiments, L-IHC methods are used to label and measure multiple biomarkers, wherein one biomarker is labeled per membrane. The measured biomarkers are scored, wherein each biomarker is designated with an assigned value. These assigned scores are based on a graded scale, which may range from zero to a higher integer designated by the user that satisfactorily segregates the measured biomarkers and is amenable to further analysis and/or biostatics. It is understood that there are many different methodologies for scoring measured biomarkers and the user and/or pathologist may devise any scoring method that satisfactorily assigns a score based on an inferred amount of measured biomarker in the patient sample comprising cancerous cells. Herein, Applicants disclose two embodiments of scoring methods (See, FIG. 1 and Example 1).

In certain embodiments, the predictive score is calculated as an aggregate score. In this instance, the assigned scores are combined to calculate an aggregate score. In some of the examples provided below, the assigned score of the most relevant biomarkers were combined by simply adding to generate an aggregate score (see e.g. Tables 4 and 6). In an alternative embodiment, some of the assigned score from a panel of biomarkers are summed and then multiplied by the assigned score of one of the biomarkers in the panel. See, Example 3 and FIG. 4B. It should be appreciated, however, that if advantageous, more sophisticated biostatistical parameters could be utilized (e.g. giving different weights to different biomarkers) as known in the art. Hence, methods are practiced to determine statistical significance, for example, using parametric or non-parametric paradigms, confidence limits and so on, and then appropriate comparisons are made to predetermined cut-off value, whether, for example, a mean, median, geometric mean and so on, so long as there is a statistical basis to conclude whether a sample is positive or negative (e.g. responsive or non-responsive).

In other embodiments, either with or without first calculating the aggregate score, the predictive score may also be based on an individual biomarker from the panel that was measured. In this instance, there may be individual biomarkers from the larger panel that measurement may be predictive alone. In this instance, an assigned score is designated to the measured biomarker, either weighted or un-weighted, may be predictive, or the assigned score may be further manipulated, such as by the calculation or a ratio.

In certain embodiments, the amount of reporter can be determined by a qualitative assessment, for example, fluorescence can be visually scored by a user on a graded zero to four scale, with zero representing no label and four representing a large amount of label. To provide a degree of normalization, to control any subjective variability between and among samples, scores can be compared or related, such as, dividing one score by a number to obtain an index. Thus, a control reagent can be run in parallel on the same sample, filter and so on, such as, a known positive and/or negative control. The scores can be averaged to yield an average or mean score for a condition or state. In this instance, an assigned score for a biomarker can be divided by the assigned score for a control tested in parallel to obtain an index and unitless value. The raw data can be transformed and manipulated into an informative, qualitative or more rapidly understandable result to the patient as a design choice.

In other embodiments, the assigned scores for measured biomarkers from a panel are neither combined to form an aggregate score nor predictive as individual biomarkers. In certain embodiments, the assigned scores for the panel of biomarkers collectively form a predictive signature score.

Regardless of how the predictive score is calculated, e.g., aggregate, individually or as a proteomic signature, these scores need to be compared to a predetermined cut off or threshold value to predict responsiveness of the therapeutic agent in question. The predetermined cut off or threshold value is calculated as described above for a panel of biomarkers and a specific disease tissue.

Once a predictive score is determined for each patient sample, based on a data set from the retrospective samples comprising a threshold value for predicting responsiveness, this information may be provided to a physician and/or oncologist. This information may be provided in a report comprising a treatment recommendation for the patient diagnosed with a particular disease. In certain embodiments, the report may comprise the prediction for responsiveness of the tumor to the targeted therapy, but not a treatment recommendation.

In certain embodiments the treatment recommendation is for a patient diagnosed with a renal cell carcinoma. In another embodiment, the treatment recommendation is for a patient diagnosed with a breast cancer, in particular HER2 positive breast cancer.

In other embodiments, the information or report provided to the physician and/or oncologist does not comprise a treatment recommendation based on the aggregate or predictive score.

In a further embodiment, the methods and systems disclosed herein can be used to increase the power and effectiveness of clinical trials. Thus, individuals determined to have a particular disease or disorder, are more likely to respond to a particular treatment modality. In a particular aspect, the methods and systems disclosed herein can be used to select subjects most likely to be responders to a particular treatment modality. In another aspect, the methods and systems disclosed herein can be used to select subjects most likely to be non-responders to a particular treatment modality.

The methods and systems disclosed herein can be used as part of suite of tools that a healthcare provider or healthcare benefits provider can apply depending, for example, on availability of samples and/or equipment, or particular preferences of doctors and/or patients.

### Computer-Implemented Methods on Computer-Readable Media

The methods disclosed herein can be implemented, in all or in part, as computer executable instructions on known computer-readable media. For example, the methods described herein can be implemented in hardware. Alternatively, the methods can be implemented in software stored in, for example, one or more memories or other computer readable medium and implemented on one or more processors. The processors can be associated with one or more controllers, calculation units and/or other units in a computer system, or implanted in firmware as desired.

When implemented in software, the software can be stored in any computer readable memory such as in RAM, ROM, flash memory, a magnetic disk, a laser disk, or other storage medium, as is also known. Likewise, this software can be delivered to a user or computer device via any known delivery method including, for example, over a communication channel such as a telephone line, the internet, a wireless connection, etc., or via a transportable medium, such as a computer readable disk, flash drive, etc.

The steps of the disclosed methods and systems are operational with numerous general or special purpose computer system environments or configurations. Examples of well-known computing systems, environments, and/or configuration that can be suitable for use with methods or systems disclosed herein include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. The methods and systems can also be practiced in distributed computing environments where tasks are performed by remote processing devices that are linked through a communications network.

Computer-readable media can be any available media that can be accessed by computer and includes both volatile and nonvolatile media, removable and nonremovable media. By way of example, and not limitation, computer readable media can comprise computer storage media and communication media. Computer storage media includes both volatile and nonvolatile, removable and nonremovable media implemented in any method or technology for storage of information such as computer readable instructions, data structures, program modules or other data. Computer storage media includes, but it is not limited to, RAM, ROM, EEPROM, flash memory or other memory technology, CD-ROM, DVD or other optical disk storage, magnetic cassettes, magnetic tape, magnetic disk storage or other magnetic storage devices, or any other medium which can be used to store the desired information and which can be accessed by computer.

The computer implemented methods and computer-readable media disclosed herein can be used by patients and/or healthcare providers and/or healthcare benefit provider as a stand-alone tool or via a server, for example, a web server. The tool can include computer-readable components, an input/output system, and one or more processing units. The input/output system can be any suitable interface between user and computer system, for input and output of data and other information, and for operable interaction with the one or more processing units. In one aspect, data to be inputted into the tool can be derived from one source, for example, a doctor or a clinical laboratory. In one aspect, data to be inputted into the tool can be derived from more than one source, for example, a doctor and a clinical laboratory. In some aspects, the input/output system can provide direct input from measuring equipment. The input/output system, in one embodiment provides an interface for a standalone computer or integrated multi-component computer system having a data processor, a memory, and a display. Data can be entered numerically, as a mathematical expression, or as a graph. In some aspects, data can be automatically or manually entered from an electronic medical record.

In some aspects, data is electronically inputted into the tool from an electronic medical record or from a clinical laboratory, healthcare provider, or healthcare benefits provider data server. In some aspects, data is outputted from the tool and electronically sent, e.g., via secure and encrypted email, to a clinical laboratory, healthcare provider, healthcare benefits provider, or patient.

In some aspects, the instructions for execution in the computer-readable medium are executed iteratively using measurements from samples collected at least one week apart. In other aspects, the instructions for execution in the computer-readable medium are executed iteratively using measurements from samples collected at least two weeks apart. In yet other aspects, the instructions for execution in the computer-readable medium are executed iteratively using measurements from samples collected at intervals disclosed elsewhere in the present disclosure.

Any methods of the present disclosure and all their variants (e.g., using different mathematical approaches to computational model construction, using different type and number of analytes, using different type and number of predictors, applications to different types of therapy and therapeutic agents, applications to different types of pulmonary diseases or disorders, etc.) can be implemented in computer-readable media and in computer systems comprising the disclosed computer-readable media and/or computer-implementations of the disclosed methods.

The present disclosure provides a computer-readable medium containing instructions for identifying a patient as a candidate for a therapy to treat a solid tumor with an mTOR pathway specific drug, wherein execution of the program instructions by one or more processors of a computer system causes the one or more processors to carry out the steps of:

(a) processing inputted data obtained from the measurement of at least one mTOR pathway biomarker in a sample taken from a patient having a solid tumor;

(b) calculating a aggregate score from the processed inputted data;

wherein the aggregate score identifies the patient as a candidate for a therapy to treat the solid tumor.

Also provided is a computer-readable medium containing instructions for identifying a candidate therapy to treat a solid tumor, wherein execution of the program instructions by one or more processors of a computer system causes the one or more processors to carry out the steps of:

(a) processing inputted data obtained from the measurement of at least one mTOR pathway biomarker in a sample taken from a patient having a solid tumor;

(b) calculating a aggregate score from the processed inputted data;

wherein the aggregate score identifies an mTOR pathway specific drug as the candidate therapy.

The instant disclosure also provides a computer-readable medium containing instructions for predicting the responsiveness or non-responsiveness of a patient to an mTOR pathway specific drug, wherein execution of the program instructions by one or more processors of a computer system causes the one or more processors to carry out the steps of:

(a) processing inputted data obtained from the measurement of at least one mTOR pathway biomarker in a sample taken from a patient having a solid tumor,

(b) calculating a aggregate score from the processed inputted data;

wherein a aggregate score above a predetermined cut off value calculated from retrospective samples predicts the responsiveness or non-responsiveness of a patient to an mTOR pathway specific drug.

Also provided is a computer-readable medium containing instructions for managing the administration of an mTOR pathway specific drug to treat a solid tumor by a healthcare provider, wherein execution of the program instructions by one or more processors of a computer system causes the one or more processors to carry out the steps of:

(a) processing inputted data obtained from the measurement of at least one mTOR pathway biomarker in a sample taken from a patient having a solid tumor;

(b) calculating a aggregate score from the processed inputted data;

wherein the aggregate score is used by the healthcare provider for managing the treatment of the solid tumor.

The present disclosure also provides a computer-readable medium containing instructions for managing the administration of an mTOR pathway specific drug to treat a solid tumor by a healthcare benefits provider, wherein execution of the program instructions by one or more processors of a computer system causes the one or more processors to carry out the steps of:

(a) processing inputted data obtained from the measurement of at least one mTOR pathway biomarker in a sample taken from a patient having a solid tumor;

(b) calculating a aggregate score from the processed inputted data;

wherein the aggregate score is used by the healthcare benefits provider for managing the treatment of the solid tumor.

In some embodiments, the sample comprises fresh, frozen, or preserved tissue, biopsy, aspirate, blood or any blood constituent, a bodily fluid, cells, or combinations thereof. In some embodiments, the bodily fluid is cerebral spinal fluid, amniotic fluid, peritoneal fluid, or interstitial fluid. In other embodiments, the sample further comprises preservatives, anticoagulants, buffers, fixatives, nutrients, antibiotics, or combinations thereof. In some specific embodiments, the samples are fixed.

In some embodiments, the method implemented in the computer-readable medium comprises using at least 2, 3, 4, 5, 6, 7, 8, 9, 10, 11, 12, 13, 14, 15, 16, 17, 18, 19 or 20 biomarkers. In some embodiments, at least one mTOR pathway biomarker is selected from the group consisting of ras, p110, p85, PI3K, PTEN, Akt, PDK1, mTOR, Rictor, Raptor, IRS1, PIP2, PIP3, Proctor, mLST8, PLD1, PA, Redd1/2, FKBP12, TSC1, FKBP38, FK506, FK520, ERK, RSK1, LKB1, Sin1, AMPK, TSC1, Rheb, PRAS40, PHLPP1/2, GSK3b, PKA, 4EBP1, eiF4E, eiF4A, FOXO1, Rag A/B/C/D, SHIP1, pAKT Substrate, TSC2, p70S6K, ATG13, 4E-BP1, PGC-1, S6K, Tel2, β-RAF, PPAR, AMPK, Dv1, HIF2α, NF1, ROC1, eIF4B, S6, eEF2K, PDCD4, various GPCR's, HIF1α, STK11, p53, SGK, PKC, TORK3, and FKBP.

In some embodiments of the method implemented in the computer-readable medium, the mTOR pathway specific drug inhibits the expression and/or activation of AKT, mTOR, pTSC2, HIF1α, pS6, p4EBP1, PI3K, or STAT3.

In some embodiments of the method implemented in the computer-readable medium, the mTOR pathway specific drug is mTOR drug is temsirolimus, everolimus, ridaforolimus, serolimus, AZD8055, or combinations thereof. In some specific embodiments, the mTOR pathway specific drug is temsirolimus.

In some embodiments of the method implemented in the computer-readable medium at least one assigned score is weighted.

In some embodiments of the method implemented in the computer-readable medium, the measurement of at least one mTOR pathway biomarker in a sample taken from a patient comprises an immunological binding assay. In some embodiments, the immunological binding assay is an enzyme linked immunosorbent assay (ELISA), an enzyme immunoassay (EIA), a radioimmunoassay (RIA), a fluoroimmunoassay (FIA), a chemiluminescent immunoassay (CLIA), a counting immunoassay (CIA), a filter media enzyme immunoassay (MEIA), a fluorescence-linked immunosorbent assay (FLISA), an agglutination immunoassays, a multiplex fluorescent. In other embodiments, the measurement of at least one mTOR pathway biomarker in a sample taken from a patient comprises immunohistochemistry (IHC). In some embodiments, the measurement of at least one mTOR pathway biomarker in a sample taken from a patient comprises immunoblotting. In some embodiments, the measurement of at least one mTOR pathway biomarker in a sample taken from a patient comprises multiplex tissue analysis. In some embodiments, the multiplex tissue analysis comprises layered immunohistochemistry (L-IHC), layered expression scanning (LES) or multiplex tissue immunoblotting (MTI).

In some embodiments of the method implemented in the computer-readable medium, the solid tumor is a kidney cancer, a breast cancer, a pancreatic cancer, a bone tissue sarcoma, or a soft tissue sarcoma. In some embodiments, the kidney cancer is renal cell carcinoma (RCC). In some embodiments, the solid tumor is a kidney tumor. In some embodiments, the measurement of at least one mTOR pathway biomarker comprises measuring p-mTOR, p4EBP1 (Ser 65) and p4EBP1 (Thr 37/46). In other embodiments, the measurement of at least one mTOR pathway biomarker consists of measuring pmTOR, p4EBP1 (Ser 65) and p4EBP1 (Thr 37/46). In some embodiments, the measurement of at least one mTOR pathway biomarker comprises measuring pmTOR (Ser2448), p4EBP1 (Ser65), p4EBP1 (Thr37-46), pPRAS40, mTOR, pAKT substrate, or a combination thereof. In some embodiments, the measurement of at least one mTOR pathway biomarker consists of measuring pmTOR (Ser2448), p4EBP1 (Ser65), p4EBP1 (Thr37-46), pPRAS40, mTOR, and pAKT substrate. In other embodiments, the measurement of at least one mTOR pathway biomarker comprises measuring CA IX, pPRAS40, mTOR, pmTOR (Ser 2448), p4EBP1 (Ser 65), p4EBP1 (Thr 37-46), 4EBP1, PRAS40, pAKT substrate, or a combination thereof. In some embodiments, the measurement of at least one mTOR pathway biomarker consists of measuring CA IX, pPRAS40, mTOR, pmTOR (Ser 2448), p4EBP1 (Ser 65), p4EBP1 (Thr 37-46), 4EBP1, PRAS40, and pAKT substrate.

In some embodiments of the method implemented in the computer-readable medium, the solid tumor is Her2 positive. In some embodiments wherein the solid tumor is Her2 positive, the measurement of at least one mTOR pathway biomarker comprises measuring PTEN, pAKT (Thr 308), pPDK1, HER4, Muc4, HER2, vimentin, pAKT (Ser 473), pmTOR, pERK1/2, p4EBP1, HIF1α, mTOR, 4EBP1, or a combination thereof. In some embodiments wherein the solid tumor is Her2 positive, the measurement of at least one mTOR pathway biomarker consists if measuring PTEN, pAKT (Thr 308), pPDK1, HER4, Muc4, HER2, vimentin, pAKT (Ser 473), pmTOR, pERK1/2, p4EBP1, HIF 1α, mTOR, and 4EBP1. In some embodiments wherein the solid tumor is Her2 positive, the measurement of at least one mTOR pathway biomarker comprises measuring pmTOR, pERK1/2, p4EBP1, HIF 1α, or a combination thereof. In some embodiments wherein the solid tumor is HER2 positive, the measurement of at least one mTOR pathway biomarker consists of measuring pmTOR, pERK1/2, p4EBP1 and HIF 1α.

In some embodiments of the method implemented in the computer-readable medium, the therapy comprises a second therapeutic agent that does not inhibit the mTOR pathway. In some embodiments, the second therapeutic agent is selected from the group consisting of trastuzumab, bevacizumab, cetuximab, imatinib, erlotinib, sunitinib, sorafenib, pazopanib, vandetanib, axitinib, aflibercept, AGM386, motesanib, cediranib, cabozantinib, tivozanib, regorafenib, ramucirumab, cilengitide, volociximab, IMC-18F, T13-403, and anti-EGFL7.

The computer-implemented methods and computer-readable media disclosed herein as equally applicable to pathways other than the mTOR pathway (e.g., the VEGF pathway), provided that the appropriate sets of biomarkers (e.g., VEGF biomarkers) and treatments (e.g., treatments comprising VEGF pathway specific drugs) are used.

### Kits

Also provided in the present disclosure is a kit for identifying cancer patients that are likely to be responders or non-responders to a therapeutic agent that inhibits a signal transduction pathway, e.g., the mTOR pathway or the VEGF pathway. The kit can comprise containers filled with nucleic acid probes (e.g., oligonucleotides) capable of hybridizing nucleic acids (e.g., mRNA) encoding the biomarkers disclosed herein or fragments thereof. In some aspects, the kit comprises container filled with reagents capable of detecting the presence of protein biomarkers disclosed herein, e.g., antibodies. In some embodiments, antibodies binding to biomarkers are detectably labeled. In other embodiments, the binding of antibodies to protein biomarkers can be detected using a secondary reagent, for example, a secondary antibody. Oligonucleotide probes and/or antibody probes can be labeled by any method known in the art, e.g., using fluorescent or radioactive labels. Oligonucleotide probes in the kit can be unlabeled. In some aspects, the kit also contains controls and/or calibration standards.

In some aspects, the kit can be used for diagnostic or investigational purposes on patient samples such as blood or a fraction thereof, muscle, skin, or a combination thereof. The kit can comprise oligonucleotides capable of hybridizing to DNA and/or RNA. Such DNA and/or RNA can be a full gene nucleic acid, or correspond to a fragment or degradation product. In some aspects, the kit can be used to detect the biomarkers disclosed herein or fragments thereof, ideally in a purified form.

Optionally associated with the kit's container(s) can be a notice in the form prescribed by a governmental agency regulating the manufacture, use or sale of pharmaceuticals or biological products, which notice reflects approval by the agency of manufacture, use or sale for human administration.

### Specific Embodiments

E1. A method of treating a patient having a solid tumor with a therapy comprising a pathway specific drug: comprising: (a) measuring two or more pathway biomarkers in a sample taken from a patient having a solid tumor to calculate an assigned score for each biomarker; (b) calculating an aggregate score from at least two assigned scores, wherein an aggregate score above a predetermined cut off value calculated from retrospective samples indicates that the patient will benefit from administration of a therapy comprising a pathway specific drug; and, (c) administering the therapy to the patient if the aggregate score indicates that the patient will benefit from the administration of the therapy.

E2. A method of treating a patient having a solid tumor with a therapy comprising a pathway specific drug comprising: (a) calculating an aggregate score from at least two assigned scores derived from the measurement of at least two pathway biomarker in a sample taken from a patient having a solid tumor; (b) determining from the aggregate score that the patient will benefit from administration of a therapy comprising a pathway specific drug if the aggregated score is above a predetermined cut off value calculated from retrospective samples; and, (c) administering the therapy to the patient if the aggregate score indicates that the patient will benefit from the administration of the therapy.

E3. A method of treating a patient having a solid tumor with a therapy comprising a pathway specific drug comprising: (a) measuring at least two pathway biomarkers in a sample taken from a patient having a solid tumor to calculate at least two assigned scores; (b) calculating an aggregate score from the at least two assigned score, wherein an aggregate score above a predetermined cut off value calculated from retrospective samples indicates whether the patient will benefit from the administration of a therapy comprising the pathway specific drug; and, (c) instructing a healthcare provide to administer the therapy to the patient if the aggregate score indicates that the patient will benefit from the administration of the therapy.

E4. A method of treating a patient having a solid tumor with a therapy comprising a pathway specific drug comprising: (a) calculating an aggregate score from at least two assigned scores derived from the measurement of at least two pathway biomarker in a sample taken from a patient having a solid tumor; (b) determining from the aggregate score that the patient will benefit from administration of a therapy comprising a pathway specific drug if the aggregated score is above a predetermined cut off value calculated from retrospective samples; and, (c) instructing a healthcare provider to administer the therapy to the patient if the aggregate score indicates that the patient will benefit from the administration of the therapy.

E5. A method of treating a patient having a solid tumor with a therapy comprising a pathway specific drug comprising: (a) determining from an aggregate score calculated from at least two assigned scores derived from the measurement of at least two pathway biomarker in a sample taken from a patient having a solid tumor, that the patient will benefit from administration of a therapy if the aggregated score is above a predetermined cut off value calculated from retrospective samples; and, (b) administering the therapy to the patient if the aggregate score indicates that the patient will benefit from the administration of the therapy.

E6. A method of treating a patient having a solid tumor with a therapy comprising a pathway specific drug comprising: (a) submitting a sample taken from a patient having a solid tumor for measurement of at least two pathway biomarker, calculation of at least two assigned scores, and determination of an aggregate score calculated from at least two assigned scores, wherein an aggregated score above a predetermined cut off value calculated from retrospective samples indicates that the patient will benefit from administration of a therapy; and, (b) administering a therapy comprising a pathway specific drug to the patient if the aggregate score indicates that the patient will benefit from the administration of the therapy.

E7. A method of treating a patient having a solid tumor with a therapy comprising a pathway specific drug comprising: (a) submitting a sample taken from a patient having a solid tumor for measurement of at least two pathway biomarker, calculation of at least two assigned scores, and determination of an aggregate score calculated from the at least two assigned scores, wherein an aggregated score above a predetermined cut off value calculated from retrospective samples indicates that the patient will benefit from administration of a therapy; and, (b) instructing a healthcare provide to administer a therapy comprising a pathway specific drug to the patient if the aggregate score indicates that the patient will benefit from the administration of the therapy.

E8. A method of determining whether a patient is in need of therapy to treat a solid tumor with a therapy comprising a pathway specific drug, comprising: (a) measuring at least two pathway biomarker in a sample taken from a patient having a solid tumor to calculate at least two assigned scores; (b) calculating an aggregate score from the at least two assigned scores, wherein a aggregate score above a predetermined cut off value calculated from retrospective samples indicates that the patient will benefit from the administration of a therapy comprising a pathway specific drug; and, (c) instructing a healthcare provide to administer the therapy if the aggregate score indicates that the patient will benefit from the administration of the therapy.

E9. A method of determining whether a patient is in need of therapy to treat a solid tumor with a therapy comprising a pathway specific drug, comprising: (a) calculating a aggregate score from at least two assigned score derived from the measurement of at least two pathway biomarkers in a sample taken from a patient having a solid tumor; (b) determining from the aggregate score whether the patient will benefit from the administration of a therapy comprising a pathway specific drug, wherein a aggregate score above a predetermined cut off value calculated from retrospective samples indicates that the patient will benefit from the administration of the therapy; and, (c) administering the therapy to the patient in need thereof.

E10. A method of determining whether a patient is in need of therapy to treat a solid tumor with a therapy comprising a pathway specific drug comprising: (a) determining from an aggregate score whether the patient will benefit from the administration of a therapy comprising a pathway specific drug, (i) wherein the aggregate score is calculated from at least two assigned scores derived from the measurement of at least two pathway biomarkers in a sample taken from a patient having a solid tumor, and (ii) wherein an aggregate score above a predetermined cut off value calculated from retrospective samples indicates that the patient will benefit from the administration of the therapy; and, (b) administering the therapy to the patient or instructing a healthcare provider to administer the therapy to the patient to treat the solid tumor.

E11. A method of determining whether a patient is in need of therapy to treat a solid tumor with a therapy comprising a pathway specific drug comprising: (a) submitting a sample taken from a patient having a solid tumor for measurement of at least two pathway biomarker, calculation of at least two assigned scores, determination of an aggregate score calculated from the at least one assigned score, or a combination thereof, (i) wherein the aggregate score is calculated from the at least two assigned scores calculated from the measurement of at least two pathway biomarker in the sample, and (ii) wherein an aggregate score above a predetermined cut off value calculated from retrospective samples indicates that the patient will benefit from the administration of the therapy; and, (b) administering the therapy to the patient or instructing a healthcare provider to administer the therapy to the patient to treat the solid tumor.

E11. A method of treating a patient having a solid tumor with a therapy comprising a pathway specific drug comprising: (a) using immunohistochemistry (IHC) to measure at least two pathway biomarker in a sample taken from a patient having a solid tumor to calculate at least two assigned scores; and, (b) calculating an aggregate score from the at least two assigned scores, wherein an aggregate score above a predetermined cut off value calculated from retrospective samples indicates that the patient will benefit from administration of a therapy comprising a pathway specific drug.

E12. The method of embodiment E11, further comprising administering the therapy to the patient if the aggregate score indicates that the patient will benefit from the administration of the therapy.

E13. A method of treating a patient having a solid tumor with a therapy comprising a pathway specific drug comprising: (a) calculating an aggregate score from at least two assigned scores derived from immunohistochemistry (IHC) measurement of at least two pathway biomarkers in a sample taken from a patient having a solid tumor; and, (b) determining from the aggregate score that the patient will benefit from administration of a therapy comprising a pathway specific drug if the aggregated score is above a predetermined cut off value calculated from retrospective samples.

E14. The method of embodiment E13, further comprising administering the therapy to the patient if the aggregate score indicates that the patient will benefit from the administration of the therapy.

E15. A method of treating a patient having a solid tumor with a therapy comprising a pathway specific drug comprising: (a) using immunohistochemistry (IHC) to measure at least two pathway biomarkers in a sample taken from a patient having a solid tumor to calculate at least two assigned score; and, (b) calculating an aggregate score from the at least two assigned scores, wherein an aggregate score above a predetermined cut off value calculated from retrospective samples indicates whether the patient will benefit from the administration of a therapy comprising the pathway specific drug.

E16. The method of claim 139, further comprising instructing a healthcare provide to administer the therapy to the patient if the aggregate score indicates that the patient will benefit from the administration of the therapy.

E17. A method of treating a patient having a solid tumor with a therapy comprising a pathway specific drug comprising: (a) calculating an aggregate score from at least two assigned scores derived from immunohistochemistry (IHC) measurement of at least two pathway biomarkers in a sample taken from a patient having a solid tumor, and, (b) determining from the aggregate score that the patient will benefit from administration of a therapy comprising a pathway specific drug if the aggregated score is above a predetermined cut off value calculated from retrospective samples.

E18. The method of embodiment E17, further comprising instructing a healthcare provider to administer the therapy to the patient if the aggregate score indicates that the patient will benefit from the administration of the therapy.

E19. A method of treating a patient having a solid tumor with a therapy comprising a pathway specific drug comprising: (a) determining from an aggregate score calculated from at least two assigned scores derived from immunohistochemistry (IHC) measurement of at least two pathway biomarker in a sample taken from a patient having a solid tumor, that the patient will benefit from administration of a therapy if the aggregated score is above a predetermined cut off value calculated from retrospective samples.

E20. The method of embodiment E19, further comprising administering the therapy to the patient if the aggregate score indicates that the patient will benefit from the administration of the therapy.

E21. A method of treating a patient having a solid tumor with a therapy comprising a pathway specific drug comprising: (a) submitting a sample taken from a patient having a solid tumor for immunohistochemistry (IHC) measurement of at least two pathway biomarkers, calculation of at least two assigned score, and determination of an aggregate score calculated from at least two assigned scores, wherein an aggregated score above a predetermined cut off value calculated from retrospective samples indicates that the patient will benefit from administration of a therapy.

E22. The method of embodiment E21, further comprising administering a therapy comprising a VEGF pathway specific drug to the patient if the aggregate score indicates that the patient will benefit from the administration of the therapy.

E23. A method of treating a patient having a solid tumor with a therapy comprising a pathway specific drug comprising: (a) submitting a sample taken from a patient having a solid tumor for immunohistochemistry (IHC) measurement of at least two pathway biomarker, calculation of at least two assigned scores, and determination of an aggregate score calculated from the at least two assigned scores, wherein an aggregated score above a predetermined cut off value calculated from retrospective samples indicates that the patient will benefit from administration of a therapy.

E24. The method of embodiment E23, further comprising instructing a healthcare provide to administer a therapy comprising a pathway specific drug to the patient if the aggregate score indicates that the patient will benefit from the administration of the therapy.

E25. A method of determining whether a patient is in need of therapy to treat a solid tumor with a therapy comprising a pathway specific drug, comprising: (a) using immunohistochemistry (IHC) to measure at least two pathway biomarker in a sample taken from a patient having a solid tumor to calculate at least one assigned score; and, (b) calculating an aggregate score from the at least two assigned scores, wherein a aggregate score above a predetermined cut off value calculated from retrospective samples indicates that the patient will benefit from the administration of a therapy comprising a pathway specific drug.

E26. The method of embodiment E25, further comprising instructing a healthcare provide to administer the therapy if the aggregate score indicates that the patient will benefit from the administration of the therapy.

E27. A method of determining whether a patient is in need of therapy to treat a solid tumor with a therapy comprising a pathway specific drug, comprising: (a) calculating an aggregate score from at least two assigned scores derived from the immunohistochemistry (IHC) measurement of at least two pathway biomarker in a sample taken from a patient having a solid tumor; and, (b) determining from the aggregate score whether the patient will benefit from the administration of a therapy comprising a pathway specific drug, wherein a aggregate score above a predetermined cut off value calculated from retrospective samples indicates that the patient will benefit from the administration of the therapy.

E28. The method of embodiment E27, further comprising administering the therapy to the patient in need thereof.

E29. A method of determining whether a patient is in need of therapy to treat a solid tumor with a therapy comprising a pathway specific drug comprising: (a) determining from an aggregate score whether the patient will benefit from the administration of a therapy comprising a pathway specific drug, (i) wherein the aggregate score is calculated from at least two assigned scores derived from the immunohistochemistry (IHC) measurement of at least two pathway biomarkers in a sample taken from a patient having a solid tumor, and (ii) wherein a aggregate score above a predetermined cut off value calculated from retrospective samples indicates that the patient will benefit from the administration of the therapy.

E30. The method of embodiment E29, further comprising administering the therapy to the patient or instructing a healthcare provider to administer the therapy to the patient to treat the solid tumor.

E31. A method of determining whether a patient is in need of therapy to treat a solid tumor with a therapy comprising a pathway specific drug comprising: (a) submitting a sample taken from a patient having a solid tumor for immunohistochemistry (IHC) measurement of at least two pathway biomarkers, calculation of at least two assigned scores, determination of a aggregate score calculated from the at least two assigned score, or a combination thereof, (i) wherein the aggregate score is calculated from the at least two assigned scores calculated from the measurement of at least two pathway biomarker in the sample, and (ii) wherein a aggregate score above a predetermined cut off value calculated from retrospective samples indicates that the patient will benefit from the administration of the therapy.

E32. The method of embodiment E31, further comprising administering the therapy to the patient or instructing a healthcare provider to administer the therapy to the patient to treat the solid tumor.

E33. The method of embodiments E1-E32, wherein the solid tumor is from a kidney cancer, a breast cancer, a pancreatic cancer, a bone tissue sarcoma, or a soft tissue sarcoma.

E34. The method of embodiment E33, wherein the kidney cancer is renal cell carcinoma (RCC).

The method of any one of embodiments E1-E33, wherein solid tumor is advanced renal cell carcinoma (RCC).

The method of any one of embodiments E1-E33, wherein solid tumor is HER2 positive breast cancer.

The method of any one of embodiments E1-E33, wherein solid tumor is HER2 negative breast cancer.

The method of interest now will be exemplified in the following non-limiting examples.

### EXAMPLES

### Example 1: Methods for Scoring Measured Biomarkers in a Sample Comprising Cancerous Cells

### A: Scoring Method Utilizing Labeling of Total Protein and Signal Transduction Pathway Biomarkers

Following patient sample acquisition (e.g. formalin-fixed paraffin-embedded (FFPE) tissue sections) the samples were prepared and membranes stained using well known L-IHC methods (described above). In particular, each biomarker was measured using a primary or secondary antibody labeled with a fluorescent dye (e.g., Cy 5) and total protein measured with fluorescent dye that was distinguished from the biomarker dye (e.g. measured in a different channel, red and green). The cancerous tissue areas were delineated from non-cancerous (e.g. normal) tissue to provide regions of interest (ROI) on an adjacent tissue section on a glass slide (See, Panel A of FIG. 1A); it is within these one or more ROI on each membrane that the biomarkers are scored. The designated assigned score for the biomarker is a sum of this scoring from each ROI. In this scoring method each biomarker fluorescent signal was visually scored within the ROI and where there was also signal present for total protein. See Panel B of FIG. 1A. When using L-IHC there are multiple stacked membranes (e.g. one for each biomarker) and this scoring method is uniformly applied to each membrane in the stack, even if for the purposes of illustration membrane and/or biomarker in the singular is referenced. After staining for both total protein, and the biomarker, the biomarker is designated with a number (e.g. zero (0) to four (4)) based on intensity of the label, wherein zero represents no measurable biomarker on the membrane and one to four represent increasing intensity of the measured biomarker that may be present in one or more ROI with labeled total protein.

As illustrated in FIG. 1A, the biomarker of interest was present in all ROI where there was total protein stained on the same membrane. The assigned score for the measured biomarker is calculated by designating a value based on the intensity of the biomarker in each region (See Panel C of FIG. 1A), multiplying that intensity designator by the percentage of the ROI area labeled with the biomarker at that intensity level (also referred to as the respective ROI or corresponding ROI). This value for each ROI is summed and if needed rounded to the nearest integer (e.g., 0 to 4). For example, in FIG. 1A each of the ROI with labeled biomarker was given a value of zero, one, two and three, respectively based on the intensity of the biomarker label. Each area was then compared to the corresponding area labeled for total protein and the designated intensity value (e.g. 0-3) was multiplied by the percentage of the area showing biomarker labeling verses total protein labeling. For example the area of biomarker labeling with an intensity of three (3) is about 5% of the total protein labeling area within all the ROIs, thus three (3) was multiplied by 0.05 to obtain 0.15. This was repeated for the other intensity levels of the biomarker wherein the two (2) intensity designator was multiplied by 0.30 for 30% of all the ROIs to obtain 0.6. In this example no other intensities were measured, but it would be possible to also have an intensity of one (1) and zero (0) when no biomarker is measured.

Next each of the numbers obtained from multiplying the intensity of the fluorescent signal by the percentage of ROI area labeled were added together to provide one assigned score for each measured biomarker (e.g., 0.15+0.0.6=0.75). Typically this number is then rounded to the nearest whole integer so that each assigned score is 0, 1, 2, 3, 4 and so on. In this instance, 0.75 is rounded to one (1) so that the assigned score is 1. See, FIG. 1A

In this methodology the intensity of signal for each measured biomarker in all ROIs is expressed as integers (0, 1, 2, 3, 4), and is derived by multiplying the fraction of the intensity represented in all ROIs with labeled biomarker, summing and rounding to the nearest integer to obtain the assigned score.

This methodology would then be repeated for each membrane in the stack for the same sample, the final number depending on the number of biomarkers being measured.

B: Scoring Method Utilizing Labeled Biomarker Intensity and a Graded Scale for ROI with Labeled Biomarker

In an alternative scoring methodology a graded scale of 0-12 was employed. This method also takes into account the intensity of the labeled biomarker and the percentage of the ROI area with labeled biomarker. The tissue sections were prepared and biomarker labeled as described in the above section of this example using L-IHC methods. In particular, following over night incubation at 4 C with a primary Antibody, the membranes were washed and incubated with a bovine anti-rabbit or mouse-biotin-Antibody for 1 hour at room temperature (RT). The membranes were washed and incubated with a second biotin-Antibody, a goat-anti bovine-IgG for 30 min at RT. The membranes were once more washed and finally incubated for 20 min with streptavidin (SA)-Cy5 at RT, washed dried and scanned

In this method the intensity of the labeled biomarker is designated based on a scale of zero (0) to three (3), with zero (0) representing no measurable labeled biomarker and three (3) representing the highest intensity of labeled biomarker. The percentage of ROI area with labeled biomarker is also designated with a graded scale from one (1) to four (4). For example, less the 10% is designated as one (1); 10% to 50% is designated as two (2); 50% to 80% is designated as three (3) and greater than 80% is designated as four (4). See FIG. 1B. In this way, a biomarker was designated with an intensity of two (2) and the percentage of the ROI area with labeled biomarker was between 50% and 80%. Thus, two (2) was multiplied by three (3) to obtain an assigned score of six (6) for the measured biomarker. In the instance where there is more than one ROI on a membrane with labeled biomarker, the assigned score for each ROI is averaged to obtain an overall assigned score for the biomarker on the membrane.

In this methodology, the intensity for each measured biomarker is expressed as an integer (e.g. 0, 1, 2, 3) and multiplied by percentage of ROI area labeled with biomarker expressed as an integer (e.g., 1, 2, 3, 4) to obtain an assigned score expressed as an integer (e.g., 0 to 12). If needed, the assigned score from each ROI on the same membrane are averaged (e.g., 6+8/2=7) to obtain an overall assigned score for the biomarker on the membrane expressed as an integer (e.g., 0 to 12).

Each of the above score methods was used on retrospective samples to obtain an assigned score and ultimately to generate a threshold value between responders and non-responders for predicting tumor responsiveness to a targeted therapy, described in detail below.

### Example 2A: Methods for Predicting Kidney Tumor Response to Sunitinib (SUTENT) Using a Panel of Five VEGF Biomarkers

In a retrospective study, a number of renal cell carcinoma (RCC) samples (biopsies, surgical specimens) were obtained from patients prior to therapy with sunitinib and whose response to therapy is known. Routinely cut formalin-fixed paraffin-embedded (FFPE) tissue sections from a total of 47 patients were received from four medical centers (Karmanos Cancer Center, Detroit, Mich.; Meir Hospital Medical Center, Tel Aviv, Israel; Shady Grove Adventist Hospital, Rockville, Md.; University of Massachusetts Cancer Center, Worcester, Mass.) and two vendors (Conversant Bio, Huntsville, Ala. and Adept Bio, Memphis, Tenn.). The samples were obtained from patients who were subsequently treated with sunitinib per standard of medical care. The information provided for each sample was limited to length of treatment with sunitinib and overall response. Thirty two (32) of the 47 patients were responders (Complete Responder [CR], Partial Responder [PR], Stable Disease [SD]) while the other 15 were non-responders [NR] to therapy as determined by radiologic, imaging and/or clinicopathologic means, or not.

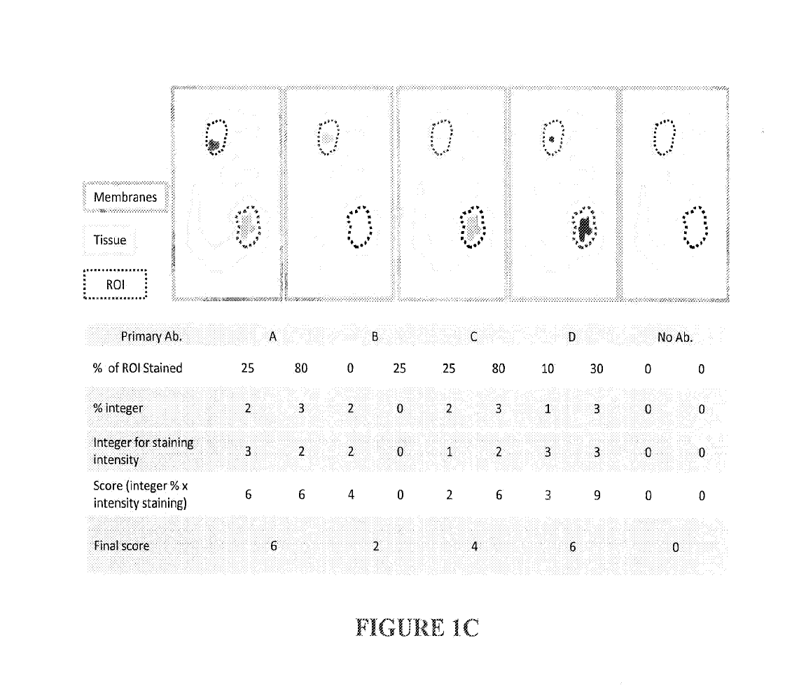

The L-IHC multiplexes were assembled using track-etched membranes of polyvinyl pyrrolidone (PVP)-coated polycarbonate (PC) film (GE Water & Process Technologies), polyvinylidene fluoride (PVDF) membrane, filter paper and ultra thick blotting paper as taught in the references listed in FIG. 2.

Antibodies were obtained either from Santa Cruz Biotechnology (r-VEGFA, sc-152, a rabbit polyclonal IgG; r-VEGFR1, sc-9029, a rabbit polyclonal IgG; m-VEGFR2 sc-6251, a goal polyclonal IgG; PDGFRβ sc-339, a rabbit polyclonal IgG); or from Cell Signaling Technology (Phospho-PRAS40 (THR 246) rabbit monoclonal antibody).

FFPE RCC tissue sections were received from five clinical centers located in the US or Israel. (Conversant Biologics, Inc., Huntsville, Ala.; Shady Grove Hospital, Rockville, Md.; Karmanos Cancer Center, Detroit, Mich.; U Mass Cancer Center, Worcester, Mass.; Meir Hospital, Kfar Saba, I L; and AdeptBio, Memphis, Tenn.). On arrival, the slides were stored at room temperature (RT) in the dark. The primary morphological characterization and identification of regions of interest (cancer, stroma, necrosis, etc.) of each specimen was performed on single H&E-stained sections.

Sections were deparaffinized and rehydrated. The sections then were incubated for 2 min in distilled water before 30 min incubation in 100 mM NH₄CO₃ pH 8.2 buffer containing 3 mM DTT at 60° C.

Digestion of kidney tissue was performed by incubation in 50 mM NH₄CO₃ pH8.2 buffer containing 10 g/ml trypsin and 2.5 μg/ml proteinase K for 15 min at 37° C. After 15 min, the slides were placed in transfer buffer for 1 min before transfer.

A stack of 10 nitrocellulose (NC)-coated polycarbonate (PC) membranes, labeled and wetted was prepared during the digestion of tissue. One PVP-coated membrane and one PVDF membrane were labeled and washed as well. The slide was removed from the transfer buffer and dried around the tissue. The PVP-coated membrane was positioned on the tissue, followed by the stack of NC-coated polycarbonate membranes, topped by the PDVF membrane. The excess of buffer/bubbles/potential wrinkles were removed by gently rolling the membranes with a sterile serological pipet. The stack was completed with three layers of 3 MM paper and two layers of thick absorbent paper. The slide was placed in transfer cassette and incubated in transfer buffer for 30 min at 55° C. followed by 2.5 h at 70° C. At the end of the transfer, the slide with the stack was placed in Tris-buffered saline (TBS) buffer and the stack was dissociated. The proteins on the PVP-coated and PVDF membranes were visualized with Ponceau red.

Each membrane was incubated overnight at 4° C. with the appropriate dilution of Abs in 3% bovine serum albumin (BSA)/TBS/0.1% Tween 20. The negative control membrane was incubated in 3% BSA/TBS/0.1% Tween 20. The next day, the membranes were washed at RT in TBS/0.1% Tween 20 twice for 15 min. The membranes then were incubated with the appropriate biotinylated-second Ab for 1 h at RT, washed twice of 15 min in TBS/0.1% Tween and incubated for an additional 30 min with biotinylated anti-second Ab species to provide an amplification of signal. After two washes, the membranes were incubated at RT for 20 min in streptavidin-Cy5, washed and dried.

The homogeneity of transfer was checked by scanning the membranes with an imager, such as, Typhoon Trio from Amersham. The ability of the membrane-captured material to autofluorescence at the same wavelength as that of FITC (λ 520 nm) was used to assess homogeneity of transfer. Membranes then were scanned with a fluorescence microscope, such as, an Olympus BX-UCB microscope at 10× (500 ms exposure for detecting Cy5 bound to the biomarker of interest; 200 ms exposure for FITC to assess total protein). See, FIG. 4C.

Regions of interest (ROIs) identified on the corresponding H & E sections were matched with the fluorescent areas detected with the Cy5 channel on each membrane. Scoring of biomarker in the cancer area of the tissue (ROI) to obtain an assigned score for each biomarker measured per sample was calculated using the method described in Example 1B. The aggregate score for each sample was then obtained by adding together each assigned score per sample. See, Tables 4A & 5A.

TABLE 4A
Advanced Renal Cell Carcinoma Retrospective Samples Treated with
SUTENT ®: 5-Panel Biomarker Set from Responder Group

| | | | | | | Aggre- |
|---|---|---|---|---|---|---|
| 2020 | SUTENT ® responders | | | | | gate |
| ID | p-PRAS40 | VEGFA | VEGFR1 | VEGFR2 | PDGFRβ | score |
| A100 | 1 | 10 | 7 | 6 | 9 | 33 |
| A103 | 3 | 9 | 3 | 2 | 0 | 17 |
| D136 | 0 | 3 | 3 | 3 | 9 | 18 |
| A107 | 2 | 4 | 6 | 1 | 6 | 19 |
| A122 | 5 | 8 | 1 | 5 | 11 | 30 |
| A126 | 2 | 6 | 8 | 3 | 3 | 22 |
| A121 | 0 | 11 | 0 | 0 | 11 | 22 |
| B101 | 0 | 10 | 6 | 5 | 11 | 32 |
| B105 | 3 | 8 | 2 | 3 | 5 | 21 |
| B106 | 4 | 6 | 2 | 2 | 6 | 20 |
| B112 | 4 | 8 | 4 | 2 | 0 | 18 |
| B115 | 3 | 11 | 5 | 1 | 11 | 31 |
| B118 | 0 | 9 | 6 | 3 | 9 | 27 |
| B119 | 1 | 7 | 4 | 0 | 7 | 19 |
| B121 | 2 | 8 | 1 | 1 | 12 | 24 |
| B122 | 0 | 11 | 1 | 2 | 11 | 25 |
| B124 | 0 | 3 | 9 | 6 | 6 | 24 |
| B128 | 12 | 10 | 2 | 0 | 0 | 24 |
| D125 | 0 | 9 | 9 | 1 | 3 | 22 |
| D128 | 1 | 9 | 8 | 1 | 12 | 31 |
| E100 | 1 | 11 | 5 | 3 | 0 | 20 |
| E101 | 0 | 9 | 5 | 4 | 10 | 28 |
| E103 | 1 | 4 | 9 | 2 | 6 | 22 |
| F102 | 0 | 6 | 4 | 0 | 12 | 22 |
| F106 | 2 | 9 | 5 | 0 | 9 | 25 |
| F108 | 2 | 6 | 3 | 1 | 12 | 24 |
| 140 + | 2 | 7 | 8 | 3 | 11 | 31 |
| 142 | | | | | | |
| F111 | 0 | 4 | 2 | 0 | 8 | 14 |
| G104 | 0 | 6 | 6 | 4 | 4 | 20 |
| F115 | 3 | 7 | 3 | 0 | 11 | 24 |
| F118 | 0 | 5 | 5 | 0 | 7 | 17 |
| F120 | 0 | 8 | 4 | 0 | 8 | 20 |

TABLE 5A
Advanced Renal Cell Carcinoma Retrospective
Samples Treated with SUTENT ®: 5-Panel
Biomarker Set from Non-Responder Group

| | | | | | | Aggre- |
|---|---|---|---|---|---|---|
| 2020 | SUTENT ® Non-Responders | | | | | gate |
| ID | p-PRAS40 | VEGFA | VEGFR1 | VEGFR2 | PDGFRβ | score |
| B100 | 1 | 12 | 4 | 0 | 7 | 24 |
| B117 | 0 | 3 | 2 | 6 | 0 | 11 |
| F114 | 0 | 4 | 2 | 1 | 9 | 16 |
| E102 | 0 | 0 | 6 | 6 | 12 | 24 |
| F100 | 0 | 3 | 2 | 0 | 9 | 14 |
| F105 | 0 | 6 | 4 | 0 | 6 | 16 |
| F110 | 0 | 3 | 2 | 2 | 8 | 15 |
| F101 | 0 | 6 | 2 | 0 | 9 | 17 |
| F104 | 0 | 3 | 2 | 0 | 8 | 13 |
| F109 | 0 | 6 | 6 | 0 | 6 | 18 |
| F116 | 0 | 0 | 0 | 0 | 2 | 2 |
| 15 + | 4 | 7 | 4 | 0 | 11 | 26 |
| 148 | | | | | | |
| F119 | 1 | 2 | 2 | 4 | 10 | 19 |
| G100 | 4 | 2 | 2 | 2 | 3 | 13 |
| G102 | 1 | 5 | 6 | 0 | 0 | 12 |

### Example 2B: Analysis of Aggregate Scores for Predicting Kidney Tumor Response to Sunitinib (SUTENT®) Using a Panel of Four VEGF Biomarkers: Impact for Patients

Assuming SUTENT is a first line treatment for advanced RCC with a 30% efficacy in that patient population (naïve and untested), Applicants analyzed the data from the retrospective data to better understand how SUTENT efficacy could be improved by selection of a patient population subset predicted to be responders and the benefit to the patient of selecting a targeted therapy based on a personalized approach to biological disease pathways.

Using the data displayed in Tables 4B & 5B (Below), the Sensitivity and Specificity was calculated at a series of cutoffs, See Table A, along with the PPV, NPV and Accuracy.

TABLE A

| | | | | True Pos. | False Neg. | True Neg | False Pos | | | |
|---|---|---|---|---|---|---|---|---|---|---|
| Cut off | Sens | Spec | 1-spec | (Get drug) | (Miss drug) | (Avoid drug) | (Get unneeded drug | PPV | NPV | Accuracy |
| 16 | 97% | 47% | 53% | 29.1 | 0.9 | 32.7 | 37.3 | 44% | 97% | 62% |
| 18 | 84% | 67% | 33% | 25.3 | 4.7 | 46.7 | 23.3 | 52% | 91% | 72% |
| 20 | 78% | 80% | 20% | 23.4 | 6.6 | 56.0 | 14.0 | 63% | 89% | 79% |
| 24 | 31% | 80% | 20% | 9.3 | 20.7 | 56.0 | 14.0 | 40% | 73% | 65% |

The cut-off was selected to maximize accuracy. Thus, a cutoff of 20 is selected, delineated in bold within Table A. As disclosed herein, the selected cutoff means that a patient with a score greater than or equal to the cutoff is predicted to be a responder, while a patient with a score below the cutoff is predicted to be a non-responder to the targeted therapy.

Assuming 100 patients with advanced RCC, in the absence of the present test, all 100 would be given SUTENT and 30 would respond. However, utilizing the present test and a patient aggregate score compared to the data set of Table 4B and 5B (below) comprising a cutoff of 20, 37 patients would be given the drug, 25 of whom would respond and 14 would not respond. In this scenario 63 patients would not be given the drug, 6.5 of whom would have responded had they been given the drug. However, when SUTENT is administered based on the present test, the efficacy of SUTENT in the responder group (those with an aggregate score of 20 or higher) would be 63%. Overall the present test and predictive algorithm would have benefited 25 patients by recommending treatment with an effective drug and helped 56 patients by recommending non-treatment with an ineffective drug. In comparison to current standard of care wherein the present test is not used and where everyone would have been treated with SUTENT, the present test and predictive algorithm have helped 56 patients by not recommending treatment with an ineffective drug, however, in this scenario, there are 6.5 patients who would have been responsive to SUTENT, but were not recommended for treatment with this drug. See Table B.

As can be seen from Table B, there are eight (8) patients that were predicted to be non-responders, who nonetheless would have responded to the targeted therapy. Thus, in certain circumstances the data may be segregated into three categories instead of two (responders and non-responders) wherein the third group would carry no prediction. In this scenario some of the predicted responders and non-responders would in fact be indeterminate with respect to a prediction. Thus, those 8 patients that are actual responders could be classified as indeterminate and would thus be considered for standard of care, which in this case is treatment with SUTENT.

TABLE 4B
Advanced Renal Cell Carcinoma Retrospective Samples Treated
with SUNITINIB: 4-Panel Biomarker Set from Responder Group

| Sample ID | VEGFA | VEGFR1 | VEGFR2 | PDGFRβ | Sum of 4 |
|---|---|---|---|---|---|
| A100 | 10 | 7 | 6 | 9 | 32 |
| B114 | 1 | 1 | 0 | 12 | 14 |
| D136 | 3 | 3 | 3 | 9 | 18 |
| A107 | 4 | 6 | 1 | 6 | 17 |
| A122 | 8 | 1 | 5 | 11 | 25 |
| A126 | 6 | 8 | 3 | 3 | 20 |
| A121 | 11 | 0 | 0 | 11 | 22 |
| B101 | 10 | 6 | 5 | 11 | 32 |
| B105 | 8 | 2 | 3 | 5 | 18 |
| B106 | 6 | 2 | 2 | 6 | 16 |
| B115 | 11 | 5 | 1 | 11 | 28 |
| B118 | 9 | 6 | 3 | 9 | 27 |
| B119 | 7 | 4 | 0 | 7 | 18 |
| B121 | 8 | 1 | 1 | 12 | 22 |
| B122 | 11 | 1 | 2 | 11 | 25 |
| B124 | 3 | 9 | 6 | 6 | 24 |
| D125 | 9 | 9 | 1 | 3 | 22 |
| D128 | 9 | 8 | 1 | 12 | 30 |
| D129 | 12 | 4 | 4 | | 20 |
| E101 | 9 | 5 | 4 | 10 | 28 |
| E103 | 4 | 9 | 2 | 6 | 21 |
| F102 | 6 | 4 | 0 | 12 | 22 |
| F106 | 9 | 5 | 0 | 9 | 23 |
| F108 | 6 | 3 | 1 | 12 | 22 |
| 140 + 142/2 | 7 | 8 | 3 | 11 | 29 |
| F111 | 4 | 2 | 0 | 8 | 14 |
| G104 | 6 | 6 | 4 | 4 | 20 |

TABLE 5B
Advanced Renal Cell Carcinoma Retrospective Samples Treated
with SUNITINIB: 4-Panel Biomarker Set from Responder Group

| Sample ID | VEGFA | VEGFR1 | VEGFR2 | PDGFRβ | Sum |
|---|---|---|---|---|---|
| B116 | 5 | 1 | 0 | | 6 |
| B100 | 12 | 4 | 0 | 7 | 23 |
| B117 | 3 | 2 | 6 | 0 | 11 |
| F114 | 4 | 2 | 1 | 9 | 16 |
| E102 | 0 | 6 | 6 | 12 | 24 |
| F100 | 3 | 2 | 0 | 9 | 14 |
| F105 | 6 | 4 | 0 | 6 | 16 |
| F107 | 6 | 2 | 0 | | 8 |
| F110 | 3 | 2 | 2 | 8 | 15 |
| F101 | 6 | 2 | 0 | 9 | 17 |
| F104 | 3 | 2 | 0 | 8 | 13 |
| F109 | 6 | 6 | 0 | 6 | 18 |
| F116 | 0 | 0 | 0 | 2 | 2 |
| F115 + 148/2 | 7 | 4 | 0 | 11 | 22 |
| F119 | 2 | 2 | 4 | 10 | 18 |
| G100 | 2 | 2 | 2 | 3 | 9 |
| G102 | 5 | 6 | 0 | 0 | 11 |

### Example 3A: Methods for Predicting Kidney Tumor Response to Sunitinib (SUTENT®) Using a Panel of Three VEGF Biomarkers

The samples were acquired and processed as described in Example 2. In this example three biomarkers were measured VEGFR1, VEGFR2 and VEGFA, instead of five in Example 2, using the reagents and methods described above.

Regions of interest (ROIs) identified on the corresponding H & E sections were matched with the fluorescent areas detected with the Cy5 channel on each membrane. Scoring of biomarkers in the cancer area of the tissue (ROI) to obtain an assigned score for each biomarker measured per sample was calculated using the method described in Example 1B. The aggregate score for each sample was then obtained by adding each assigned score for VEGFR1 and VEGFR2 together; this summed value was then multiplied by the assigned score of VEGFA. See, Table 4C and 5C for the assigned scores (aggregate score=(VEGFR1+VEGFR2)*VEGFA) and FIG. 4B for plot of these assigned scores.

TABLE 4C
Advanced Renal Cell Carcinoma Retrospective Samples Treated with
SUTENT ®: 3-Panel Biomarker Set from Responder Group

| | | | | Aggregate |
|---|---|---|---|---|
| SUTENT ® Responders | | | | score |
| 2020 ID | VEGFA | VEGFR1 | VEGFR2 | Product |
| A100 | 10 | 7 | 6 | 130 |
| A103 | 9 | 3 | 2 | 45 |
| D136 | 3 | 3 | 3 | 18 |
| A107 | 4 | 6 | 2 | 32 |
| A122 | 8 | 1 | 5 | 48 |
| A126 | 4 | 6 | 2 | 32 |
| A121 | 11 | 0 | 0 | 0 |
| B101 | 10 | 6 | 5 | 110 |
| B105 | 8 | 2 | 3 | 40 |
| B106 | 6 | 2 | 2 | 24 |
| B112 | 8 | 4 | 2 | 48 |
| B115 | 11 | 5 | 1 | 66 |
| B118 | 9 | 6 | 3 | 81 |
| B119 | 7 | 4 | 0 | 28 |
| B121 | 8 | 1 | 1 | 16 |
| B122 | 8 | 1 | 2 | 24 |
| B124 | 3 | 9 | 6 | 45 |
| B128 | 10 | 2 | 0 | 20 |
| D125 | 9 | 9 | 1 | 90 |
| D128 | 9 | 8 | 1 | 81 |
| D129 | 12 | 4 | 4 | 96 |
| E100 | 11 | 5 | 3 | 88 |
| E101 | 9 | 5 | 4 | 81 |
| E103 | 4 | 9 | 2 | 44 |
| F102 | 6 | 4 | 0 | 24 |
| F106 | 9 | 5 | 0 | 45 |
| F108 | 6 | 3 | 1 | 24 |
| 140 + 142 | 7 | 8 | 3 | 77 |
| G104 | 6 | 6 | 4 | 60 |
| F111 | 4 | 2 | 0 | 8 |
| F115 | 7 | 3 | 0 | 21 |
| F118 | 5 | 5 | 0 | 25 |
| F120 | 8 | 4 | 0 | 32 |

TABLE 5C
Advanced Renal Cell Carcinoma Retrospective
Samples Treated with SUTENT ®: 3-Panel
Biomarker Set from Non-Responder Group

| | | | | Aggregate |
|---|---|---|---|---|
| SUTENT ® Non-Responders | | | | Score |
| 2020 ID | VEGFA | VEGFR1 | VEGFR2 | Product |
| A105 | 4 | 3 | 1 | 16 |
| B102 | 6 | 2 | 2 | 24 |
| B116 | 5 | 1 | 0 | 5 |
| E102 | 0 | 6 | 6 | 0 |
| F100 | 3 | 2 | 0 | 6 |
| F101 | 6 | 2 | 0 | 12 |
| F104 | 3 | 2 | 0 | 6 |
| F105 | 6 | 4 | 0 | 24 |
| F107 | 6 | 2 | 0 | 12 |
| F109 | 6 | 6 | 0 | 36 |
| F110 | 3 | 2 | 2 | 12 |
| F113 | 3 | 3 | 0 | 9 |
| F114 | 4 | 2 | 1 | 12 |
| F116 | 0 | 0 | 0 | 0 |
| F119 | 2 | 5 | 5 | 20 |
| F124 | 7 | 2 | 0 | 14 |
| G100 | 3 | 1 | 0 | 3 |
| G102 | 2 | 2 | 0 | 4 |

### Example 3B: Analysis of Aggregate Scores for Predicting Kidney Tumor Response to Sunitinib (SUTENT® Using a Panel of Three VEGF Biomarkers: Impact for Patients

Assuming SUTENT is a first line treatment for advanced RCC with a 30% efficacy rate in that patient population (naïve and untested), Applicants analyzed the data from the retrospective data to better understand how SUTENT efficacy could be improved by selection of a patient population subset predicted to be responders and the benefit to the patient of selecting a targeted therapy based on a personalized approach to biological disease pathways.

Using the data displayed in Tables 4C & 5C (above), the Sensitivity and Specificity was calculated at a series of cutoffs, See Table C, along with the PPV, NPV and Accuracy.

TABLE C

| | | | True Pos. | False Neg. | True Neg | False + | | | |
|---|---|---|---|---|---|---|---|---|---|
| Cut off | Sens. | Spec. | (Get drug) | (Miss drug) | (Avoid drug) | (Get unneeded drug | PPV | NPV | Accuracy |
| 16 | 94% | 72% | 28.2 | 1.8 | 50.5 | 19.5 | 59% | 97% | 78.7% |
| 20 | 91% | 78% | 27.3 | 2.7 | 54.5 | 15.5 | 64% | 95% | 81.7% |
| 24 | 82% | 83% | 24.5 | 5.5 | 58.3 | 11.7 | 68% | 91% | 82.9% |
| 28 | 67% | 94% | 20.0 | 10.0 | 66.1 | 3.9 | 84% | 87% | 86.1% |
| 32 | 64% | 94% | 19.1 | 10.9 | 66.1 | 3.9 | 83% | 86% | 85.2% |

The cut-off was selected to maximize accuracy. Thus, a cut off of 24 is selected, with a sensitivity of 82% and a specificity of 83%, yielding a positive predictive value (PPV) of 68% and a negative predictive value (NPV) of 91%, delineated in bold within Table D.

Assuming 100 patients with advanced RCC, in the absence of the present test, all 100 would be given SUTENT and 30 would respond. However, utilizing the present test and predictive algorithm, a patient aggregate score compared to the data set of Table 4C and 5C comprising a cutoff of 24, 24.5 patients would be selected for SUTENT therapy, 19 of whom would respond. In this scenario, 77 patients would not be recommended for SUTENT treatment, which would be the correct course of action for 66 of these patients. The efficacy of SUTENT in the predicted responder group is 83%. In comparison to current standard of care wherein in the absence of the present test everyone would receive SUTENT treatment the present tested have helped 66 patients by recommending non-treatment with an ineffective drug, however, in this scenario, there are 11 patients who would have been responsive to SUTENT, but were not recommended for treatment with this drug. See Figure D

As can be seen from Table D, there are six (6) patients that were predicted to be non-responders, who nonetheless would have responded to the targeted therapy. Thus, in certain circumstances the data may be segregated into three categories instead of two (responders and non-responders) wherein the third group would carry no prediction. In this scenario some of the predicted responders and non-responders would in fact be indeterminate with respect to a prediction. In this instance, the indeterminate group may have the same 30% chance of responding to SUTENT as they did before the Test. Those six patients would fall into the indeterminate group in this scenario. See, FIG. 7.

In another scenario, the data may be analyzed and segregated into four scoring categories. See Table E.

TABLE E

| | | | | Non- | % non- | | |
|---|---|---|---|---|---|---|---|
| | Responders | % Responders | 100 | Responders | responders | Non- | |
| | per range | per range | patients | per range | per range | resp/100 pts | PPV |
| X >= 32 | 21 | 63.6% | 19.1 | 1 | 5.6% | 3.9 | 83% |
| 24 <= X < 32 | 6 | 18.2% | 5.5 | 2 | 11.1% | 7.8 | 39% |
| 16 <= X < 24 | 4 | 12.1% | 3.6 | 2 | 11.1% | 7.8 | 34% |
| X < 16 | 2 | 6.1% | 1.8 | 13 | 72.2% | 50.5 | 4% |
| Total | 33 | 100.0% | 30 | 18 | 100.0% | 70 | |

Based on the data from Tables 4C and 4B, if 100 patients with advanced RCC were tested with the present methods, 23 patients would have scores at or above 32, nineteen (19.1) of which would be responders to SUTENT (83% accuracy). Thirteen (13) patients would have a score 24<=<32, with a 41% chance of responding to SUTENT. Eleven (11.4) patients would have a score 16 to <24, with a 31% chance of being a responder. Fifty-two (52.3) patients would have a score below 16, with only a 3% chance of being a responder to SUTENT.

### Example 4: Methods for Predicting Kidney Tumor Response to mTOR Inhibitor (TORISEL® or AFINITOR) Using a Panel of Six mTOR Biomarkers

In a retrospective study, a number of renal cell carcinoma (RCC) samples (biopsies, surgical specimens) were obtained from patients prior to therapy with temsirolimus and whose response to therapy is known. Routinely cut formalin-fixed paraffin-embedded (FFPE) tissue sections from a total of 33 patients were received from three medical centers (Karmanos Cancer Center, Detroit, Mich.; Meir Hospital Medical Center, Tel Aviv, Israel; University of Massachusetts Cancer Center, Worcester, Mass.) and two vendors (Conversant Bio, Huntsville, Ala. and Adept Bio, Memphis, Tenn.). The samples were obtained from patients who were subsequently treated with temsirolimus per standard of medical care. The information provided for each sample was limited to length of treatment with sunitinib and overall response. Twelve of the 33 patients were responders (Complete Responder [CR], Partial Responder [PR], Stable Disease [SD]) while the other 21 were non-responders [NR] to therapy as determined by radiologic, imaging and/or clinicopathologic means, or not.

On arrival, the slides were stored at room temperature (RT) in the dark. The primary morphological characterization and identification of regions of interest (cancer, stroma, necrosis etc.) of each specimen was performed on single H&E-stained sections.

The L-IHC experiments were performed using track-etched membranes of polyvinyl pyrrolidone (PVP)-coated polycarbonate (PC) film (GE Water & Process Technologies), polyvinylidene fluoride (PVDF) membrane, filter paper and ultra thick blotting paper as taught in the references hereinabove.

Antibodies were obtained commercially, indicated by antigen detected, p-4E-BP1 thr 37/46 (Cell Signaling Technologies #2855); p-4E-BP1 S65 (Cell Signalling Technologies #9451); PRAS40 (Cell Signaling Technologies #2691); mTor (Cell Signaling Technologies #2983); p-mTor Ser2448 (Cell Signaling Technologies #2971); and p-AKT substrate (Cell Signaling Technologies #9614).

Sections were rehydrated by successive washes in increasing diluted baths of ethanol (from 100% to 70%). The sections then were incubated for 2 min in distilled water before 30 min incubation in 100 mM NH₄CO₃ pH 8.2 buffer containing 3 mM DTT at 60° C.

Digestion of kidney tissue was performed by incubation in 50 mM NH₄CO₃ pH8.2 buffer containing 10 μg/ml trypsin and 2.5 μg/ml proteinase K for 15 min at 37° C. After 15 min, the slides were placed in transfer buffer (25 mM Tris, 192 mM Glycine pH8.3) for 2 min before transfer.

A stack of 10 nitrocellulose (NC)-coated polycarbonate (PC) membranes, labeled and wetted was prepared during the digestion of tissue. One PVP-coated membrane and one PVDF membrane were labeled and were washed as well. The slide was removed from the transfer buffer and dried around the tissue. The PVP-coated membrane was positioned on the tissue, followed by the stack of NC-coated polycarbonate membranes, topped by the PDVF membrane. The excess of buffer/bubbles/potential wrinkles were removed by gently rolling the membranes with a sterile serological pipet. The stack was completed with three layers of 3 MM paper and two layers of thick absorbent paper. The slide was placed in transfer cassette and incubated in transfer buffer for 30 min at 55° C. followed by 2.5 h at 70° C. At the end of the transfer, the slide with the stack was placed in Tris-buffered saline (TBS) buffer and the stack was dissociated. The proteins on the PVP-coated and PVDF membranes were visualized with Ponceau red.

Each membrane was incubated overnight at 4° C. with the appropriate dilution of Abs in 3% bovine serum albumin (BSA)/TBS/0.1% Tween 20. The control membrane was incubated in 3% BSA/TBS/0.1% Tween 20. The next day, the membranes were washed at RT in TBS/0.1% Tween 20 twice for 15 min. The membranes were then incubated with the appropriate commercially available biotinylated-secondary Ab for 1 h at RT, washed twice of 15 min in TBS/0.1% Tween and incubated for an additional 30 min with a commercially available biotinylated anti-secondary Ab antibody. After two washes, the membranes were incubated at RT for 20 min in commercially available streptavidin-Cy5, washed and dried.

The homogeneity of transfer was checked by scanning the membranes with an imager, such as, Typhoon Trio from Amersham. The ability of the membrane-captured material to fluoresce at the same wavelength as that of FITC (λ 520 nm) was used to assess background. Membranes were then scanned with a fluorescence microscope, such as, an Olympus BX-UCB microscope at 10× (500 ms exposure for Cy5, 200 ms exposure for FITC).

Regions of interest (ROIs) identified on the corresponding H & E sections were matched with the fluorescent areas detected with the Cy5 channel on each membrane. Scoring of biomarker in the cancer area of the tissue (ROI) to obtain an assigned score for each biomarker measured per sample was calculated using the method described in Example 1A. The aggregate score for each sample was then obtained by adding together each assigned score per sample. See, Table 6; FIGS. 5A and 5C.

Samples from a total of 33 patients, including 12 responders and 21 non-responders, scored for nine markers using an appropriate negative control, which is selected as a design choice. For example, a negative control may be obtained using an irrelevant primary antibody or no primary antibody on a filter or membrane. The scores of six markers relative to the selected negative control that showed the most statistically significant differences between responders and non-responders were obtained for mTOR, p-mTOR_Ser 2448, p-4EBP1_Ser 65, p-4EBP1_Thr 37-46, PRAS40 and p-AKT_Substrate.

### Example 5: Methods for Predicting Kidney Tumor Response to an mTOR Inhibitor (TORISEL® or AFINITOR) Using a Panel of Three mTOR Biomarkers

The samples were acquired and processed as described in Example 4. In this example three biomarkers were measured, pmTOR (Ser 2448), p4EBP1 (Ser 65), p4EBP1 (Thr 37-46), instead of six in Example 4, using the reagents and methods described above.

Regions of interest (ROIs) identified on the corresponding H & E sections were matched with the fluorescent areas detected with the Cy5 channel on each membrane. Scoring of biomarkers in the cancer area of the tissue (ROI) to obtain an assigned score for each biomarker measured per sample was calculated using the method described in Example 1A. The aggregate score for each sample was then obtained by adding together each biomarker assigned score per sample. See, Table 6 where the three marker subset is indicated with the grey field and FIG. 5B.

### Discussion

The mTOR pathway, a key regulator of cell proliferation, is often found dysregulated in the numbers of cancer (See, Example 6 below) contributing to tumorigenesis. In 2007 TORISEL was approved for the treatment of RCC by the FDA and EMEA. mTOR inhibitors have also been shown to be effective in treatment of other tumors, such as Glioblastoma multiforme (Galanis E., et al. J Clinical Oncology 2005; 23:5294-5304), but have yet to gain regulatory approval. In the EU, TORISEL is approved as a first-line therapy for advanced RCC. However, in the US while the FDA has approved TORISEL for treatment of advanced RCC, it has not been approved for a specific line of treatment. In fact, a recent clinical trial of TORISEL by Pfizer for treating RCC failed to reach end-points as a second line therapy (2012). Patient selection may have played a role in the missed end points as prior data indicates this drug should be effective in treating RCC. Nonetheless, in the US when patients with RCC fail anti-angiogenic therapy (e.g., SUTENT) mTOR inhibitors (TORISEL or AFINITOR) are generally used as second line of treatment, due in part to a paucity of targeted therapies for the disease. Failing a VEGF inhibitor treatment though may not be enough to select the patients that will respond to an mTOR inhibitor. Especially since, depending on the clinical trial, only between about 8% and 33% of patients diagnosed with RCC and treated with an mTOR inhibitor are responsive to the drug and show some degree of efficacy (stable disease or partial responder using standard guidelines). Demonstrating expression of protein in the mTOR pathway, and thus inferred activation of the pathway, would provide a useful tool for selecting those patients with advanced RCC that would have a better chance of being responsive to the drug.

Here, using the multiplex IHC, we investigate the expression and activation of protein of the mTOR pathway that could help discriminate patients that would respond or not to treatment with mTOR inhibitors. Upstream regulators (such as AKT substrates) of mTOR as well as downstream effectors (such as 4E-BP1) were investigated.

Using the data displayed in Table 6, plots were generated using either 6 or 3 of the biomarkers selected (FIGS. 5A and 5B).

FIG. 5A shows that with a 6 biomarker panel and a cut off of 10, it was possible to accurately detect 7 out of 12 (58%) responders and 17 out of 21 (81%) of non-responders. Interestingly, when a 3 markers panels was used (FIG. 5 B), the percentage of correctly identified responders reached 75% with a cut off of 6, while still identifying 17 out of 21 (81%) of non-responders. These results demonstrate a dramatic improvement in selecting patients who will be responders to an mTOR inhibitor over the absence of a test and may be used to select patients for first-line therapy with an mTOR inhibitor.

### Example 6A: Methods for Predicting HER2 Positive Breast Cancer Non-Responsiveness to HERCEPTIN® Using a Panel of Four mTOR Biomarkers and an Aggregate Score

In a retrospective study, layered immunohistochemistry (L-IHC) technology was used to examine a number of HER2+ breast cancer tissue samples (biopsies, lumpectomies and mastectomies) obtained from patients prior to therapy and whose response to therapy is known. Routinely cut FFPE tissue sections (10) from a total of 45 patients were received from the pathology archives of two medical centers (Meir Hospital Medical Center, Tel Aviv, Israel and Beebe Medical Center, Lewes, Del.) and a single vendor (Conversant Bio, Huntsville, Ala.). The samples were obtained from patients who were subsequently treated per standard of medical care and included HERCEPTIN in conjunction with chemotherapy. Of the 45 samples, 32 are from patients who were responders (complete (CR) or partial (PR) responders) and 13 are from patients who were non-responders to treatment. Treatment response was ascertained, by radiologic imaging, laboratory and/or clinicopathologic means within the respective clinical center that the patient was treated.

Two tissue sections were used to probe a total of fourteen different markers (7 markers per tissue section). The L-IHC multiplexes were assembled using track-etched membranes of polyvinyl pyrrolidone (PVP)-coated polycarbonate (PC) film (GE Water & Process Technologies), polyvinylidene fluoride (PVDF) membrane, filter paper and ultra thick blotting paper as taught in the references listed in FIG. 2.

Antibodies were obtained from Santa Cruz Biotechnology (PTEN, p-AKT (T308), p-PDK1, HER4, MUC4, HER2, vimentin, p-AKT (S473), p-mTOR, p-ERK, p-4EBP1, HIF1-alpha, mTOR, 4EBP1).

Tissue slides were deparaffinized in three changes of NEO-CLEAR® solvent (for 5 minutes each) and rehydrated through a graded alcohol series (from 100% to 70%) to distilled water. Slides were then treated with 3 mM DTT (G-Bioscience) in 50 mM NH₄HCO₃ buffer, pH 8.2 (Teknova) for 30 min. at 60° C.,

To perform digestion of breast tissues, slides were treated with an enzyme cocktail solution (20 μg/mL trypsin (Sigma), 0.002% proteinase-K (Dako), 50 mmol/L NH₄HCO₃, pH 8.2) for 15 min at 37° C. The slides were subsequently washed 3 times in Tris-Glycine transfer buffer (Quality Biologicals).

The proteins from treated slides were transferred to an 8-membrane stack of P-films (20/20 GeneSystems) as described below. The slides were laid out on the clean surface with tissue sections facing up, and covered with PE membrane (Track-Etched Polyester PETE Membranes, GE Water & Process Technologies) soaked in the transfer buffer. Subsequently, PE membrane was covered with an 8 P-film membrane stack. The assembly was completed with placing on the top of the stack an additional PE membrane spacer, one Nitrocellulose membrane (Protran 0.45 um pore size, BA85, Whatman) and then one piece of 3M filter paper (Whatman) and 2 pieces of blotting paper (BioRad) all soaked in the transfer buffer. The stack was covered with one plane glass slide, one piece of soaked in the transfer buffer blotting paper, and covered with a second glass slide. The assembly was placed in a transfer cassette while avoiding lateral shifts within the stack.

The transfer was performed in a water bath under the following conditions: incubation for 35 min. at 55° C., then for 2 hrs at 72° C. After the transfer the membrane stack was carefully disassembled in 1×PBS buffer and P-film membranes were washed in PBS (3×5 min). NC membrane and two PE spacer membranes were stained using the Blot FastStain Kit to monitor transfer quality.

Protein biotinylation was performed using EZ-Link Sulfo-NHS-biotin (Thermo Pierce) solution in 1×PBS. P-film membranes were incubated with biotin solution for 10 min. at room temperature. Following biotinylation procedure P-film membranes were washed with TBST buffer (3×5 minutes).

Blocking step was performed by P-film membranes incubation in 1×TBS-T with 0.5% BSA for 10 min. at RT. The membranes were then washed with TBST buffer 1×5 minutes.

Following washing step, the membranes were incubated with primary antibodies: p-AKT_T308, pAKT_S473, pPDK1 (S241), Muc4, PTEN (Abcam), pmTOR_S2448, mTOR, pERK1/2, p4EBP1, 4E BP1, HER4 (Cell Signaling), HER2, Vimentin (Dako), and HIF1α (Novus) overnight at 4° C. or 2 hrs at RT.

Subsequently, the membranes were incubated with ALEXA FLUOR 647 conjugated anti-rabbit or anti-mouse IgG (Jackson ImmunoResearch) for 45 min at RT. Finally, membranes were incubated with streptavidin-linked ALEXA FLUOR 488 (Jackson ImmunoResearch) for 15 min. at RT.

After staining, the membranes were washed in TBST buffer (2×15 min.), dried, individually mounted on slides, and scanned in an Olympus scanner under appropriate and consistent scanning conditions.

Another tissue section was H&E stained in which regions of interest (ROIs) were identified and were matched with the fluorescent areas detected with the Cy5 channel on each membrane. Scoring of biomarkers in the cancer area of the tissue (ROI) to obtain an assigned score for each biomarker measured per sample was calculated using the method described in Example 1A. Of these 14 biomarkers, the four (p-mTOR, pERK1/2, p-4EBP1, and HIF1α) that each most significantly correlated with responder and non-responder were used to obtain an aggregate score for each sample by adding together each biomarker assigned score per sample. See, Tables 7 and 8; and FIGS. 6A and 6B.

TABLE 7
Breast Cancer Retrospective Samples Treated with
HERCEPTIN: 4-Panel Biomarker Set from Complete Responder
(CR) and Partial Responder (PR) Group

| | | | | | | Aggre- |
|---|---|---|---|---|---|---|
| Re- | Sample | Biomarkers | | | | gate |
| sponders | ID | p-mTOR | pERK1/2 | p4EBP1 | HIF1α | Score |
| CR | D102 | 2 | 0 | 2 | 2 | 6 |
| CR | C100 | 3 | 3 | 3 | 3 | 12 |
| CR | C103A | 0 | 1 | 0 | 2 | 3 |
| CR | A109 | 0 | 1 | 0 | 0 | 1 |
| CR | D111 | 0 | 0 | 0 | 0 | 0 |
| CR | D114 | 0 | 0 | 0 | 0 | 0 |
| CR | A110 | 0 | 0 | 0 | 0 | 0 |
| CR | A111 | 0 | 0 | 0 | 0 | 0 |
| CR | A112 | 0 | 0 | 0 | 0 | 0 |
| CR | A113 | 0 | 0 | 0 | 0 | 0 |
| CR | A114 | 0 | 0 | 0 | 0 | 0 |
| CR | A116 | 1 | 2 | 0 | 3 | 6 |
| CR | D120 | 1 | 1 | 0 | 1 | 0 |
| CR | D124 | 1 | 1 | 2 | 0 | 4 |
| CR | A117 | 1 | 1 | 3 | 2 | 7 |
| CR | A118 | 0 | 0 | 0 | 1 | 1 |
| CR | A119 | 0 | 0 | 0 | 0 | 0 |
| PR | D110 | 0 | 0 | 0 | 0 | 0 |
| PR | D101 | 0 | 1 | 0 | 0 | 1 |
| PR | D103 | 1 | 0 | 1 | 1 | 3 |
| PR | D104 | 0 | 0 | 0 | 0 | 0 |
| PR | D105 | 2 | 1 | 0 | 0 | 3 |
| PR | D106 | 1 | 1 | 0 | 0 | 2 |
| PR | D107 | 0 | 0 | 0 | 0 | 0 |
| PR | D100 | 0 | 0 | 0 | 1 | 1 |
| PR | C101 | 2 | 3 | 3 | 3 | 11 |
| PR | D116 | 0 | 1 | 2 | 0 | 3 |
| PR | A108 | 2 | 3 | 3 | 3 | 11 |
| PR | D118 | 0 | 0 | 0 | 3 | 3 |
| PR | D122 | 0 | 0 | 0 | 0 | 0 |
| PR | D132 | 0 | 0 | 0 | 1 | 1 |
| PR | D133 | 0 | 0 | 0 | 0 | 0 |
| MEAN | | 0.53 | 0.63 | 0.59 | 0.81 | 2.47 |

TABLE 8
Breast Cancer Retrospective Samples Treated with HERCEPTIN:
4-Panel Biomarker Set from Non-Responder Group

| Sample | Biomarkers | | | | Aggregate |
|---|---|---|---|---|---|
| ID | p-mTOR | pERK1/2 | p4EBP1 | HIF1α | Score |
| D109 | 2 | 0 | 3 | 3 | 8 |
| D108 | 2 | 3 | 3 | 3 | 11 |
| C102 | 2 | 2 | 3 | 3 | 10 |
| C104 A | 3 | 3 | 2 | 4 | 12 |
| D113 | 2 | 3 | 2 | 0 | 7 |
| D112 | 1 | 2 | 0 | 0 | 3 |
| D115 | 2 | 3 | 3 | 2 | 10 |
| D119 | 2 | 3 | 3 | 3 | 11 |
| D121 | 0 | 0 | 0 | 0 | 0 |
| D117 | 0 | 0 | 0 | 1 | 4 |
| A120 | 2 | 3 | 4 | 4 | 13 |
| D131 | 3 | 3 | 1 | 1 | 8 |
| D134 | 3 | 2 | 0 | 2 | 7 |
| MEAN | 1.85 | 2.08 | 1.85 | 2.00 | 8.00 |

### Example 6B: Methods for Predicting HER2 Positive Breast Cancer Non-Responsiveness to HERCEPTIN® Using a Panel of Four mTOR Biomarkers and an Index Score

The samples were acquired and processed as described in Example 6A. In this example four biomarkers were measured, p-mTOR (Ser 2448), pERK, p4EBP1, HIF1α, using the reagents and methods described above.

Regions of interest (ROIs) identified on the corresponding H & E sections were matched with the fluorescent areas detected with the Cy5 channel on each membrane. Scoring of biomarkers in the cancer area of the tissue (ROI) to obtain an assigned score for each biomarker measured per sample was calculated using the method described in Example 1A. In the responder group, the scores for each of the fourteen markers that were tested on 32 patients were averaged to yield a mean binding value. The same occurred for 13 patients that were found to be non-responders to HERCEPTIN treatment, see Table 7 and 8 with the scores.

The mean scores for each marker then were related to yield an index value, that is, the mean value for the non-responder group was divided by the mean value for the responder group to yield an index value. That index value can be used to obtain a threshold value for identifying a potential non-responder and responder. Hence, as noted in Table 9 below, an index value for any one marker above 2 could be considered as diagnostic that the candidate in not likely to respond to HERCEPTIN treatment.

TABLE 9

| Marker | Index |
|---|---|
| pAKT_T308 | 1.35 |
| pPDK1 | 1.23 |
| HER4 | 0.95 |
| MUC4 | 1.58 |
| HER2 | 1.54 |
| Vimentin | 0.67 |
| pAKT_S473 | 1.48 |
| p-mTOR | 3.48 |
| pERK1/2 | 3.32 |
| p4EBP1 | 3.11 |
| HIF1A | 2.46 |

The stained images for some of the markers examined in one patient are provided in FIG. 2. The various membranes are arranged consecutively. In the bottom row are images that infer the protein content on the membrane as revealed by general biotin staining. The amount of transferred proteins diminishes with the more distal membranes. Individual membranes then were exposed to a particular antibody which specifically binds a marker. The first membrane depicts a negative control with no specific antibody. Membranes two through eight each were exposed to an antibody that specifically binds PTEN, pAKT (T308), pPDK1 (S241), HER4, MUC4, HER2 and vimentin, respectively.

A scatter plot of the patients is provided in FIG. 6C.

The results suggest that analysis of those four markers improves prediction of patient response to trastuzumab as compared to HER2 alone, and could suggest a potential response to additional therapy with an mTOR-targeted therapy for non-responders.

The receiver operating characteristic (ROC) curve was calculated with an area under the curve of 0.81 (95% confidence interval of 0.6733 to 0.9637). See, FIG. 6D. A calculated cut off value to differentiate responders and non-responders to trastuzumab is 6.5 with a sensitivity of 87.5% (correct responder prediction of 28 out of 32 cases (95% confidence interval of 0.7101 to 0.9649) and specificity of 72.9% (correct non-responder prediction of 10 out of 13 cases, 95% confidence interval of 0.4619 to 0.9496). A prediction accuracy of 81.25 is about 2˜4-fold better than assays commonly used to detect HER2 alone with 18-35% responder predictions.

### Discussion

The results suggest a substantial improvement in prediction of patients who will be responders to trastuzumab from 40% HER2 alone to 82% using the 4 protein panel. Since non-responders show increased expression of these biomarkers along the mTOR pathway, this suggests bypass resistance to the HER2-based therapy and could indicate benefit of these patients with the addition of an mTOR inhibitor to their treatment.

The ability to measure mTOR pathway activity in tumor tissue may have broad clinical applicability. Dysregulation of the mTOR pathway creates a favorable environment for the development and progression of many cancers, including breast cancer, and is associated with the development of resistance to endocrine therapy and to the anti-human epidermal growth factor receptor-2 (HER2) monoclonal antibody trastuzumab. Therefore, the addition of mTOR inhibitors to conventional breast cancer therapy has the potential to enhance therapeutic efficacy and/or overcome innate or acquired resistance. Everolimus, an mTOR inhibitor with demonstrated preclinical activity against breast cancer cell lines, has been shown to reverse Akt-induced resistance to hormonal therapy and trastuzumab. Phase I-II clinical trials have demonstrated that everolimus has promising clinical activity in women with HER2-positive, HER2-negative, and estrogen receptor-positive breast cancer when combined with HER2-targeted therapy, cytotoxic chemotherapy, and hormonal therapy, respectively. Everolimus is currently under evaluation in a series of phase III Breast Cancer Trials of Oral Everolimus (BOLERO) trials of women with HER2-positive and estrogen receptor-positive breast cancer. Results of these trials will help to establish the role of everolimus in the treatment of clinically important breast cancer subtypes (Pharmacotherapy. 2012 April; 32(4):383-96). An assay to stratify patients could have large impact on the standard of care.

All references cited herein are herein incorporated by reference in entirety.

1-36. (canceled)

37. A method of treating a patient having a solid tumor with a therapy comprising a pathway specific drug comprising:
(a) obtaining a sample from a patient having a solid tumor;
(b) measuring levels of two or more pathway biomarkers in the sample;
(c) calculating a predictive score from the measured pathway biomarker levels;
(d) comparing the predictive value to a threshold value, calculated from retrospective samples, to determine whether or not it is above or below the threshold value;
(e) concluding, if the probability value is above the threshold value, that the patient will likely benefit from administration of a therapy comprising a pathway specific drug;
(f) concluding, if the probability value is below the threshold value, that the patient will likely not benefit from administration of a therapy comprising a pathway specific drug; and,
(g) administering the pathway specific drug to the patient with a probability value above the threshold value.

38. The method of claim 37, wherein the solid tumor is advanced renal cell carcinoma (RCC).

39. The method of claim 37, wherein the solid tumor is HER2 positive breast cancer.

40. The method of claim 37, wherein the solid tumor is HER2 negative breast cancer.

41. The method of claim 37, wherein the pathway specific drug inhibits an mTOR or VEGF signal transduction pathway.

42. The method of claim 37, wherein the pathway biomarker is a VEGF pathway biomarker selected from mTOR, p-mTOR (Ser 2448), p-mTOR (Ser 2481), AKT, pAKT (ser 473), pAKT (substrate), PI3K, TSC1, pTSC (Thr 1462), TSC2, pTSC2 (Ser 939), PRAS40, pPRAS40 (Thr 246), pPRAS40 (Ser 183), 4EBP1, p4EBP1 (Ser 65), p4EBP1 (Thr 3746), Rictor, pRictor (Thr 1135), HIF1α, HIF1β, HIF2α, VEGFA, VEGFR1, VEGFR2, pVEGFR2 (Tyr 996), pVEGFR2 (Tyr 1175), VEGFB, PDGFRα, PDGFRβ, CAIX, CD31, CD34, EGFR, Integrin αV, Integrin α6, FAK, PIGF, Vimentin, ERK, pERK, Raf-B, Raf-1, Raptor, S6 Ribosomal protein, pS6 Ribosomal protein (Ser235/236), p70 S6 Kinase, p70 S6 Kinase, (Thr389), p70 S6 Kinase (Ser371), VHL (von Hippel-Lindau), pEGFR (Tyr 845), pHER2 (Tyr1248)/EGFR (Tyr1173), pHER2 (Tyr 1248), pHER2 (Tyr 1221/1222), pFAK (Tyr 397).

43. The method of claim 42, wherein the VEGF pathway biomarker is selected from VEGFA, VEGFR1, VEGFR2, p-PRAS40, VEGFB, HIF1α, HIF1β, HIF2α, PDGFRα and PDGFRβ.

44. The method of claim 42, wherein the VEGF pathway biomarker is selected from VEGFR1, VEGFR2 and VEGFA.

45. The method of claim 44, wherein the pathway specific drug is SUTENT.

46. The method of claim 37, wherein the at least one pathway biomarker is a mTOR pathway biomarker selected from the group consisting of ras, p110, p85, p13K, PTEN, Akt, PDK1, mTOR, Rictor, Raptor, IRS1, PIP2, PIP3, Proctor, mLST8, PLD1, PA, Redd1/2, FKBP12, TSC1, FKBP38, FK506, FK520, ERK, RSK1, LKB1, Sin1, AMPK, TSC1, Rheb, PRAS40, PHLPP1/2, GSK3b, PKA, 4EBP1, eiF4E, eiF4A, FOXO1, Rag A/B/C/D, SHIP1, pAKT Substrate, TSC2, p70S6K, ATG13, 4E-BP1, PGC-1, S6K, Tel2, BRAF, PPAR, AMPK, Dv1, HIF1α, NF1, ROC1, eIF4B, S6, eEF2K, PDCD4, various GPCR's, HIF1α, STK11, p53, SGK, PKC, TORK3, and FKBP.

47. The method of claim 46, wherein the at least one pathway biomarker is for measuring, in HER2 positive breast cancer, mTOR pathway biomarkers selected from the group consisting of mTOR, p-mTOR (Ser 2448), pPTEN, AKT, pAKT (ser 473), pAKT (Thr 308), PI3K, 4EBP1, p4EBP1 (Thr 37/46), HIF1α, Vimentin, HER2, HER4, MUC4, PDK, pPDK (Ser 241), ERK, pERK (Thr 202/Tyr 204), and Actin.

48. The method of claim 46, wherein the at least one pathway biomarker is for measuring, in renal cell carcinoma, mTOR pathway biomarkers selected from the group consisting of mTOR, p-mTOR (Ser 2448), p-4EBP1 (Ser 65), p-4EBP1 (Thr 37/46), PRAS40, and p-AKT (Substrate).

49. The method of claim 46, wherein the pathway specific drug is mTOR drug temsirolimus, everolimus, ridaforolimus, serolimus, AZD8055, or combinations thereof.

50. The method of claim 37, wherein the threshold value is a sensitivity value of 50% or greater based on a specificity value of about 80%.

51. The method of claim 37, wherein the threshold value is a sensitivity value of 80% or greater based on a specificity value of about 80%.
